(19) United States
(12) Patent Application Publication
(10) Pub. No.: US 2016/0297891 A1
(43) Pub. Date: Oct. 13, 2016

(54) METHODS USING MONOVALENT ANTIGEN BINDING CONSTRUCTS TARGETING HER2

(71) Applicant: Zymeworks Inc., Vancouver (CA)

(72) Inventors: Gordon Yiu Kon Ng, Vancouver (CA); Nina E. Weisser, Delta (CA); Grant Raymond Wickman, Vancouver (CA)

(21) Appl. No.: 15/036,175

(22) PCT Filed: Nov. 13, 2014
(86) PCT No.: PCT/US14/65571

§ 371 Date: May 12, 2016

Related U.S. Application Data
(60) Provisional application No. 61/903,839, filed on Nov. 13, 2013.

(51) Int. Cl. C07K 16/32; A61K 39/395; C07K 16/30

(52) U.S. Cl. CPC C07K 16/32; C07K 16/3069; A61K 39/39558; A61K 2039/507

(57) ABSTRACT

Provided herein are methods of use and treatment using a first or a first and second monovalent antigen-binding constructs targeting HER2. The monovalent antigen-binding constructs can include at least one antigen-binding polypeptide comprising a heavy chain variable domain, wherein the antigen-bind polypeptide specifically binds HER2; and a heterodimeric Fc, the Fc comprising at least two CH3 sequences, wherein the Fc is coupled, with or without a linker, to the antigen-binding polypeptide.

### CROSS REFERENCE TO RELATED APPLICATIONS

This application claims the benefit of U.S. Provisional Application No. 61/903,839, filed Nov. 13, 2013, which is hereby incorporated in its entirety by reference.

### SEQUENCE LISTING

The instant application contains a Sequence Listing which has been submitted via EFS-Web and is hereby incorporated by reference in its entirety. Said ASCII copy, created on Nov. 13, 2014, is named 27905PCT_sequencelisting.txt, and is 355,986 bytes in size.

### BACKGROUND

In the realm of therapeutic proteins, antibodies with their multivalent target binding features are excellent scaffolds for the design of drug candidates. Current marketed antibody therapeutics are bivalent monospecific antibodies optimized and selected for high affinity binding and avidity conferred by the two antibody FABs. Defucosylation or enhancement of FcgR binding by mutagenesis have been employed to render antibodies more efficacious via antibody Fc dependent cell cytotoxicity mechanisms. Afucyosylated antibodies or antibodies with enhanced FcgR binding still suffer from incomplete therapeutic efficacy in clinical testing and marketed drug status has yet to be achieved for any of these antibodies.

Therapeutic antibodies would ideally possess certain minimal characteristics, including target specificity, biostability, bioavailability and biodistribution following administration to a subject patient, and sufficient target binding affinity and high target occupancy and antibody binding to target cells to maximize antibody dependent therapeutic effects. There has been limited success in efforts to generate antibody therapeutics that possess all of these minimal characteristics, especially antibodies that can fully occupy targets at a 1:1 antibody to target ratio. For example, traditional bivalent monospecific IgG antibodies cannot fully occupy targets at a 1:1 ratio even at saturating concentrations. From a theoretical perspective, at saturating concentrations a traditional monospecific bivalent antibody is expected to maximally binds targets at a ratio of 1 antibody:2 targets owing to the presence of two identical antigen binding FABs that can confer avidity effects compared to monovalent antibody fragments. Further, such traditional antibodies suffer from more limited bioavailability and/or biodistribution as a consequence of greater molecular size. Furthermore, traditional antibodies may in some cases exhibit agonistic effects upon binding to a target antigen, which is undesired in instances where the antagonistic effect is the desired therapeutic function. In some instances, this phenomenon is attributable to the “cross-linking” effect of a bivalent antibody that when bound to a cell surface receptor promotes receptor dimerization that leads to receptor activation. Additionally, traditional bivalent antibodies suffer from limited therapeutic efficacy because of limited antibody binding to target cells at a 1:2 antibody to target antigen ratio at maximal therapeutically safe doses that permit antibody dependent cytotoxic effects or other mechanisms of therapeutic activity.

Monovalent antibodies that bind HER2 have been described in International Patent Publication Nos. WO 2008/131242 (Zymogenetics, Inc.) and WO 2011/147982 (Genmab A/S). Co-owned patent applications PCT/CA2011/001238, filed Nov. 4, 2011, PCT/CA2012/050780, filed Nov. 2, 2012, PCT/CA2013/00471, filed May 10, 2013, and PCT/CA2013/050358, filed May 8, 2013 describe therapeutic antibodies. Each is hereby incorporated by reference in their entirety for all purposes.

### SUMMARY

Disclosed herein are methods of treating a subject, e.g., a human, by administering an effective amount of a first monovalent antigen-binding construct, e.g., antibody, or a combination of a first and a second monovalent antigen-binding construct to the subject, the first and second monovalent antigen-binding constructs each having an antigen-binding polypeptide construct and a dimeric Fc coupled, with or without a linker, to the antigen-binding polypeptide construct. Each antigen-binding polypeptide construct specifically binds a extracellular domain 2 (ECD2) of human epidermal growth factor receptor 2 (HER2), a ECD4 of HER2, or a ECD1 of HER2. The first monovalent antigen-binding construct and the second monovalent antigen-binding construct bind to non-overlapping epitopes and do not compete with each other for binding to HER2.

In various embodiments, the method of treating a subject includes, for example, inhibiting growth of a HER2+ tumor, delaying progression of a HER2+ tumor, treating a HER2+ cancer or preventing a HER2+ cancer. The HER2+ tumor or cancer can be breast, ovarian, stomach, gastroesophageal junction, endometrial, salivary gland, head and neck, lung, brain, kidney, colon, colorectal, thyroid, pancreatic, prostate or bladder tumor or cancer.

In some embodiments, the monovalent antigen-binding constructs used in the methods described herein include a heterodimeric Fc comprising at least two CH3 sequences and the dimerized CH3 sequences have a melting temperature (Tm) of about 68° C. or higher. In some embodiments, the monovalent antigen-binding constructs used in the methods described herein selectively and/or specifically binds HER2 with a greater maximum binding (Bmax) as compared to a monospecific bivalent antigen-binding construct that specifically binds HER2, and wherein at a monovalent antigen-binding construct to target ratio of 1:1 the increase in Bmax relative to the monospecific bivalent antigen-binding construct is observed at a concentration greater than the observed equilibrium constant (KD) of the constructs up to saturating concentrations.

### BRIEF DESCRIPTION OF THE DRAWINGS

FIG. 1 depicts schematic representations of different OA antibody formats. FIG. 1A depicts the structure of a bivalent mono-specific, full-sized antibody, where the light chains are shown in white, the Fab portion of the heavy chain is shown in hatched fill, and the Fc portion of the heavy chains are grey. FIG. 1B depicts two versions of a monovalent, mono-specific OA where the antigen-binding domain is in the Fab format. In both of these versions, the light chain is shown in white, while the Fab portion of the heavy chain is shown in hatched fill. The Fc portion of Chain A is grey and the Fc portion of Chain B is black. In the version on the left, the Fab is fused to Chain A, while in the version on the right, the Fab is fused to Chain B. FIG. 1C depicts two versions of an OA where the antigen-binding domain is in the scFv format. In both of these versions, the variable domain of the light chain (VL) is shown in white, while the variable domain of the heavy chain (VH) is shown in hatched fill. The Fc portion of Chain A is grey and the Fc portion of Chain B is black. In the version on the left, the scFv is fused to Chain A, while in the version on the right, the scFv is fused to Chain B.

FIG. 2 depicts the ability of monovalent anti-HER2 antibody constructs to bind to ovarian HER2 2-3+(gene amplified) SKOV3 cells as measured by FACS.

FIG. 3 shows the ability of monovalent anti-HER2 antibodies to inhibit the growth of HER2-expressing breast cancer cells. FIG. 3A shows the ability of various monovalent anti-HER2 antibodies and controls to inhibit the growth of BT-474 cells. FIG. 3B shows the ability of various monovalent anti-HER2 antibodies and controls to inhibit the growth of SKOV3 cells.

FIG. 4 depicts the internalization efficiency of monovalent anti-HER2 antibodies and combinations to be internalized in SKOV3 cells.

FIG. 5 depicts the ability of monovalent anti-HER2 antibodies and combinations to mediate concentration dependent ADCC in SKOV3 cells.

FIG. 6 depicts the ability of monovalent anti-HER2 antibody ADCs to mediate in a concentration dependent manner cellular cytotoxicity. FIG. 6A depicts the ability of monovalent anti-HER2 antibody ADCs to mediate cellular cytotoxicity in SKOV3 cells. FIG. 6B depicts the ability of monovalent anti-HER2 antibody ADCs to mediate cellular cytotoxicity in JIMT1 cells.

FIG. 7 depicts the ability of monovalent anti-HER2 antibody ADCs to mediate concentration dependent cellular cytotoxicity in JIMT1 cells compared to a T-DM1 analog (v6246).

FIG. 8A depicts the ability of monovalent anti-HER2 antibody combinations to inhibit established ovarian SKOV3 tumor growth in a mouse xenograft model. FIG. 8B depicts the effect of monovalent anti-HER2 antibody combinations on survival in this model.

FIG. 9 depicts the ability of a monovalent anti-HER2 antibody to inhibit established primary breast tumor (trastuzumab and chemotherapy resistant) growth in a primary breast cancer xenograft model.

FIG. 10 depicts the pharmacokinetic profile of an exemplary monovalent antigen binding construct in mice.

FIG. 11 shows a schematic representation of the in vitro blood brain barrier model. Immortal rat brain microvascular endothelial cells (SV-ARBEC) form a tight barrier mimicking the blood brain barrier.

FIG. 12 compares the ability of OA-HER2 to transcytose the BBB compared to FSA-HER2. FIG. 12A depicts the antibody transcytosis fold increase in the in vitro BBB model compared to non-specific IgG control (n=3). FIG. 12B shows transcytosis of v1040 compared to FSA-HER2. Bars represent the mean AUC of the bottom chamber antibody concentration following normalization to the A20.1 non-specific control (n=3. *, p<0.05).

FIG. 13 shows v1040 shows increased distribution to the brain compared to v506. Bars represent average ex vivo brain fluorescence 24 hours after a 10 mg/kg injection of fluorescently labeled antibody (n=1).

FIG. 14 shows v1040 has increased distribution to the lung compared to v506. Bars represent average ex vivo lung fluorescence 24 hours after a 10 mg/kg injection of fluorescently labeled antibody (n=1).

FIG. 15 shows ex vivo quantification of lung metastasis in animals bearing HBCx-13b patient derived xenograft. Points represent individual animals with the median indicated by line (n=4).

FIG. 16 shows the ability of monovalent anti-HER2 antibodies to mediate ADCC in HER2+ cells. FIG. 16A depicts ADCC activity in SKBR3 cells; FIG. 16B depicts ADCC activity in ZR-75-1 cells; FIG. 16C depicts ADCC activity in MCF7 cells; and FIG. 16D depicts ADCC activity in MDA-MB-231 cells.

### DETAILED DESCRIPTION

Described herein are methods of treating a HER2+ cancer, comprising administering one or more monovalent antigen-binding constructs that monovalently bind HER2 (monovalent anti-HER2 antigen-binding constructs, monovalent anti-HER2 antibodies). Each monovalent anti-HER2 antigen-binding construct binds to an epitope of HER2 that is located in extracellular domain 1 (ECD1), extracellular domain 2 (ECD2), or extracellular domain 4 (ECD4). In one embodiment, more than one monovalent anti-HER2 antigen-binding construct is administered, and the monovalent anti-HER2 antigen-binding constructs are selected such that they do not bind overlapping epitopes, or block each other from binding to HER2. In some embodiments, more than one monovalent anti-HER2 antigen-binding construct is administered, and at least one of the monovalent anti-HER2 antigen-binding constructs are conjugated to a drug or toxin, such as, for example, a maytansinoid. In another embodiment, all of the monovalent anti-HER2 antigen-binding constructs administered are conjugated to a drug or toxin.

Monovalent anti HER2 antigen-binding constructs suitable for use in the methods described herein exhibit greater maximum binding Bmax to target cells expressing HER2, compared to a reference bivalent monospecific anti-HER2 antigen-binding construct (e.g. a corresponding full size antibody, FSA). Monovalent anti-HER2 antigen-binding constructs also exhibit properties in vitro, such as i) the ability to inhibit cancer cell growth; ii) the ability to kill cancer cells, iii) the ability to be internalized in cancer cells, iv) the ability to down-regulate HER2, and/or v) the ability to mediate effector cell-directed cell killing. In some embodiments, a suitable monovalent anti-HER2 antigen-binding construct exhibits increased Bmax coupled with increased growth inhibition and/or effector cell-directed cell killing compared to a reference bivalent monospecific anti-HER2 antigen-binding construct, and in some embodiments, a combination of monovalent anti-HER2 antigen-binding constructs exhibits increased Bmax coupled with increased growth inhibition and/or effector cell-directed cell killing compared to the combination of reference bivalent monospecific anti-HER2 antigen-binding constructs. The monovalent anti-HER2 antigen-binding constructs also exhibit increased tissue distribution compared to the reference bivalent monospecific anti-HER2 antigen-binding constructs.

Thus, in one embodiment, there is described a method of treating a HER2+ cancer comprising administering one or more monovalent anti-HER2 antigen-binding constructs, where the HER2+ cancer is selected from breast, ovarian, stomach, gastroesophageal junction, endometrial, salivary gland, brain, lung, kidney, colon, colorectal, thyroid, pancreatic, prostate, bladder cancer, and head and neck cancer. In another embodiment, the HER2+ cancer is selected from breast, ovarian, brain, and lung cancer.

In other embodiments, the breast cancer is refractory or resistant to trastuzumab, a chemotherapy resistant breast cancer, a triple-negative breast cancer, an estrogen receptor-negative breast cancer, or an estrogen receptor-positive breast cancer.

The increase in Bmax for target cells expressing HER2, compared to a reference bivalent monospecific anti-HER2 antigen-binding construct, as well as the ability to mediate ADCC of the monovalent anti-HER2 antigen-binding constructs are observed independent of the level of expression of HER2, however, in one embodiment, the greatest difference in ADCC activity between the monovalent anti-HER2 antigen-binding constructs and the reference bivalent mono-specific anti-HER2 antigen-binding constructs is observed in HER2+ cells that express HER2 at the 0-2+ level, where the HER2 expression level is assessed by immunohistochemistry (IHC). Thus, in one embodiment, there is described herein, a method of treating a HER2+ cancer comprising administering one or more monovalent anti-HER2 antigen-binding construct, where the HER2+ cancer expresses HER2 at the 2+ level or lower. In one embodiment, the HER2+ cancer expresses HER2 at the 1+ level.

In one embodiment, the HER2+ cancer is an ovarian cancer that expresses HER2 at the 2+/3+ level, as assessed by IHC. In one embodiment, the HER2+ cancer is a breast cancer that expresses HER2 at the 2+ or lower level, as measured by IHC. In one embodiment, the HER2+ cancer is a breast cancer that expresses HER2 at the 1+ level, as measured by IHC.

Monovalent anti-HER2 antigen-binding constructs suitable for use in the method described herein exhibit additional differences compared to reference bivalent anti-HER2 antigen-binding constructs. For example, the monovalent anti-HER2 antigen-binding constructs show increased blood-brain-barrier (BBB) permeability compared to reference bivalent anti-HER2 antigen-binding constructs, and are able to reduce the number of lung metastases in and in vivo model. Thus, described herein is a method of treating a HER2+ cancer, comprising administering one or more monovalent anti-HER2 antigen-binding construct, wherein the HER2+ cancer is an established primary and metastatic breast cancer. In one embodiment, the HER2+ cancer is a lung metastasis or brain metastasis of a primary breast cancer.

### Methods of Treatment

Described herein are methods of treating a subject. The method comprises administering to the subject an effective amount of one or more monovalent antigen-binding constructs that bind HER2.

In some embodiments, the method of treatment is for inhibiting growth of a HER2+ tumor, and/or delaying progression of a HER2+ tumor, and/or treating a HER2+ cancer or and/or preventing a HER2+ cancer. The HER2+ tumor or cancer can be breast, ovarian, stomach, gastroesophageal junction, endometrial, salivary gland, head and neck, lung, brain, kidney, colon, colorectal, thyroid, pancreatic, prostate or bladder. In some embodiments, the method is treating a HER+ breast cancer that is a trastuzumab-resistant breast cancer, a chemotherapy-resistant breast cancer, a triple-negative breast cancer, an estrogen receptor-negative breast cancer, or a estrogen receptor-positive breast cancer. In some embodiments, the method is treating or preventing a HER2+ metastatic cancer that is a metastatic breast cancer, metastatic brain cancer or a metastatic lung cancer, an established primary and metastatic breast cancer, or a lung metastasis or brain metastasis of a breast cancer.

In some embodiments, the HER2+ tumor or cancer expresses HER2 at a 2+ level or lower. In some embodiments, the HER2+ tumor or cancer is an ovarian cancer that expresses HER2 at a 2+ or 3+ level, as determined by immunohistochemistry (IHC) and as described herein.

The methods of treatment described herein comprise administration of a monovalent antigen-binding construct or a combination of monovalent antigen-binding constructs that bind to HER2. In one embodiment, the method comprises administration of two monovalent antigen-binding constructs that bind to HER2. In another embodiment, the method comprises administration of three monovalent antigen-binding constructs that bind to HER2. In still another embodiment, the method comprises administration of three or more antigen-binding constructs that bind to HER2.

When a combination of monovalent antigen-binding constructs is used, the monovalent antigen-binding constructs are selected such that they bind to non-overlapping epitopes or compete with each other for binding to HER2. For example, a combination of monovalent antigen-binding constructs can be used where each monovalent antigen-binding construct binds to ECD1, ECD2, or ECD3 of HER2. Thus in one embodiment, the combination comprises a monovalent antigen-binding construct that binds to ECD1 of HER2, and one that binds to ECD2 of HER2. In one embodiment, the combination comprises a monovalent antigen-binding construct that binds to ECD1 and one that binds to ECD4. In one embodiment, the combination comprises a monovalent antigen-binding construct that binds to ECD2 and one that binds to ECD4. In one embodiment, the combination comprises a monovalent antigen-binding construct that binds to ECD1, one that binds to ECD2, and one that binds to ECD4.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, myeloma (e.g., multiple myeloma), hepatocellular cancer, gastrointestinal cancer, pancreatic cancer, glioblastoma/glioma (e.g., anaplastic astrocytoma, glioblastoma multiforme, anaplastic oligodendroglioma, anaplastic oligodendroastrocytoma), cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishing of any direct or indirect pathological consequences of the disease, preventing metastasis, decreasing the rate of disease progression, amelioration or palliation of the disease state, and remission or improved prognosis. In some embodiments, antibodies described herein are used to delay development of a disease or disorder. In one embodiment, antibodies and methods described herein effect tumor regression. In one embodiment, antibodies and methods described herein effect inhibition of tumor/cancer growth.

The term “subject” as used herein, refers to an animal, in some embodiments a mammal, and in other embodiments a human, who is the object of treatment, observation or experiment. An animal may be a companion animal (e.g., dogs, cats, and the like), farm animal (e.g., cows, sheep, pigs, horses, and the like) or a laboratory animal (e.g., rats, mice, guinea pigs, and the like).

In some embodiments, the subject has a disorder. Examples include any condition that would benefit from treatment with an monovalent antigen-binding construct or method described herein. This includes chronic and acute disorders or diseases including those pathological conditions which predispose the mammal to the disorder in question. Non-limiting examples of disorders to be treated herein include malignant and benign tumors; non-leukemias and lymphoid malignancies; neuronal, glial, astrocytal, hypothalamic and other glandular, macrophagal, epithelial, stromal and blastocoelic disorders; and inflammatory, immunologic and other angiogenesis-related disorders.

The term “effective amount” as used herein refers to that amount of monovalent antigen-binding construct being administered, which will relieve to some extent one or more of the symptoms of the disease, condition or disorder being treated. Compositions containing the construct described herein can be administered for prophylactic, enhancing, and/or therapeutic treatments.

The terms “enhance” or “enhancing” means to increase or prolong either in potency or duration a desired effect. Thus, in regard to enhancing the effect of drug molecule or therapeutic agents, the term “enhancing” refers to the ability to increase or prolong, either in potency or duration, the effect of therapeutic agents on a system. An “enhancing-effective amount,” as used herein, refers to an amount adequate to enhance the effect of another therapeutic agent or drug in a desired system. When used in a patient, amounts effective for this use will depend on the severity and course of the disease, disorder or condition, previous therapy, the patient's health status and response to the drugs, and the judgment of the treating physician.

### Treatment of Cancers

Described herein is the use of at least one monovalent antigen-binding construct described herein for the manufacture of a medicament for treating a subject. In certain embodiments is use of a monovalent antigen-binding construct described herein for the manufacture of a medicament for inhibiting growth of a HER2+ tumor, delaying progression of a HER2+ tumor, treating a HER2+ cancer or preventing a HER2+ cancer, e.g., a breast, ovarian, stomach, gastroesophageal junction, endometrial, salivary gland, head and neck, lung, brain, kidney, colon, colorectal, thyroid, pancreatic, prostate or bladder HER2+ tumor or cancer.

In some embodiments, use of a monovalent HER2 binding antigen-binding construct described herein for the manufacture of a medicament is for treating cancer or any proliferative disease associated with EGFR and/or HER dysfunction, including HER1 dysfunction, HER2 dysfunction, HER 3 dysfunction, and/or HER4 dysfunction. In certain embodiments, the cancer is a low EGFR and/or HER2 expressing cancer. In certain embodiments, the cancer is resistant to treatment with a bivalent HER2 antibody.

In some embodiments, HER2 binding monovalent antigen-binding constructs described herein are used in the treatment of a breast cancer cell.

In some embodiments, a HER2 binding monovalent antigen-binding construct described herein is used to treat patients that are partially responsive to current therapies. In some embodiments, HER2 binding monovalent antigen-binding constructs described herein are used to treat patients that are resistant to current therapies. In another embodiment, HER2 binding monovalent antigen-binding constructs described herein are used to treat patients that are developing resistance to current therapies. In some embodiments, the use of a monovalent HER2 binding antigen-binding construct described herein for the manufacture of a medicament is for treating cancers resistant to treatment with Trastuzumab.

In one embodiment, HER2 binding monovalent antigen-binding constructs described herein are useful to treat patients that are unresponsive to current therapies for breast cancer. In certain embodiments, these patients suffer from a triple negative cancer. In some embodiments, the triple-negative cancer is a breast cancer with low to negligible expression of the genes for estrogen receptor (ER), progesterone receptor (PR) and HER2. In certain other embodiments the HER2 binding monovalent antigen-binding constructs described herein are provided to patients that are unresponsive to current therapies, optionally in combination with one or more current anti-HER2 therapies for, e.g., treatment of breast cancer. In some embodiments the current anti-HER2 therapies include, but are not limited to, anti-HER2 or anti-HER3 monospecific bivalent antibodies, trastuzumab, pertuzumab, T-DM1, a bi-specific HER2/HER3 scFv, or combinations thereof. In one embodiment, a monovalent antigen-binding construct described herein is used to treat patients that are not responsive to trastuzumab, pertuzumab, T-DM1, anti-HER2, or anti-HER3, alone or in combination.

In one embodiment, a HER2 binding monovalent antigen-binding construct that comprise an antigen-binding polypeptide construct that binds HER2 can be used in the treatment of patients with metastatic breast cancer. In one embodiment, a HER2 binding monovalent antibody is useful in the treatment of patients with locally advanced or advanced metastatic cancer. In one embodiment, a HER2 binding monovalent antibody is useful in the treatment of patients with refractory cancer. In one embodiment, a HER2 binding monovalent antibody is provided to a patient for the treatment of metastatic cancer when said patient has progressed on previous anti-HER2 therapy. In one embodiment, a HER2 binding monovalent antibody described herein can be used in the treatment of patients with triple negative breast cancers. In one embodiment, a HER2 binding monovalent antibody described herein is used in the treatment of patients with advanced, refractory HER2-amplified, heregulin positive cancers.

The HER2 binding monovalent antigen-binding constructs can be administered in combination with other known therapies for the treatment of cancer. In accordance with this embodiment, the monovalent antigen-binding constructs can be administered in combination with other monovalent antigen-binding constructs or multivalent antibodies with non-overlapping binding target epitopes to significantly increase the Bₘₐₓ and antibody dependent cytotoxic activity above FSAs. For example, monovalent HER2 binding antigen-binding constructs described herein can be administered in combination as follows: 1) a monovalent antigen-binding construct such as v1040 or v1041 in combination with v4182 (based on pertuzumab); 2) v1041 or v1040 and/or v4182 in combination with cetuximab bivalent EGFR antibody; and 3) multiple combinations of non-competing antibodies directed at the same and different surface antigens on the same target cell. In certain embodiments, the monovalent antigen-binding constructs described herein are administered in combination with a therapy selected from Herceptin™, T-DM1, afucosylated antibodies or Perj eta for the treatment of patients with advanced HER2 amplified, heregulin-positive breast cancer. In a certain embodiment, a monovalent antigen-binding construct described herein is administered in combination with Herceptin™ or Perj eta in patients with HER2-expressing carcinomas of the distal esophagus, gastroesophageal (GE) junction and stomach.

By HER2+ cancer is meant a cancer that expresses HER2 such that the monovalent antigen binding constructs described herein are able to bind to the cancer. As is known in the art, HER2+ cancers express HER2 at varying levels. To determine ErbB, e.g. ErbB2 expression in the cancer, various diagnostic/prognostic assays are available. In one embodiment, ErbB2 overexpression may be analyzed by IHC, e.g. using the HERCEPTEST® (Dako). Parrafin embedded tissue sections from a tumor biopsy may be subjected to the IHC assay and accorded a ErbB2 protein staining intensity criteria as follows: Score 0 no staining is observed or membrane staining is observed in less than 10% of tumor cells.

Score 1+a faint/barely perceptible membrane staining is detected in more than 10% of the tumor cells. The cells are only stained in part of their membrane.

Score 2+a weak to moderate complete membrane staining is observed in more than 10% of the tumor cells.

Score 3+a moderate to strong complete membrane staining is observed in more than 10% of the tumor cells.

Those tumors with 0 or 1+ scores for ErbB2 overexpression assessment may be characterized as not overexpressing ErbB2, whereas those tumors with 2+ or 3+ scores may be characterized as overexpressing ErbB2.

Alternatively, or additionally, fluorescence in situ hybridization (FISH) assays such as the INFORM™ (sold by Ventana, Ariz.) or PATHVISION™ (Vysis, Ill.) may be carried out on formalin-fixed, paraffin-embedded tumor tissue to determine the extent (if any) of ErbB2 overexpression in the tumor. In comparison with IHC assay, the FISH assay, which measures HER2 gene amplification, seems to correlate better with response of patients to treatment with HERCEPTIN®, and is currently considered to be the preferred assay to identify patients likely to benefit from HERCEPTIN® treatment or treatment with the bi-specific antibody constructs of the present invention.

In some embodiments, use of a monovalent HER2 binding antigen-binding construct described herein for the manufacture of a medicament is for treating a cancer that expresses HER2 at the 2+ level or lower, where the level of HER2 is measured by IHC. In some embodiments, use of a monovalent HER2 binding antigen-binding construct described herein for the manufacture of a medicament is for treating a cancer that expresses HER2 at the 1+ level or lower, where the level of HER2 is measured by IHC. In some embodiments, use of a monovalent HER2 binding antigen-binding construct described herein for the manufacture of a medicament is for treating a cancer that expresses HER2 at the 3+ level, where the level of HER2 is measured by IHC. In some embodiments, use of a monovalent HER2 binding antigen-binding construct described herein for the manufacture of a medicament is for treating a cancer that expresses HER2 at the 2+ level or 3+ level, where the level of HER2 is measured by IHC.

Combination Administration:

In some embodiments, use of a monovalent HER2 antigen-binding construct can be administered in combination with an additional agent (e.g. radiation therapy, chemotherapeutic agents, hormonal therapy, immunotherapy and anti-tumor agents).

### Antigen Binding Constructs

The methods of treatment described herein include administration of at least one monovalent antigen binding construct, e.g., at least one monovalent antibody, that binds to HER2. The antigen binding constructs used in the methods described herein include an Fc and an antigen binding polypeptide construct.

The term “antigen binding construct” refers to any agent, e.g., polypeptide or polypeptide complex capable of binding to an antigen. In some aspects an antigen binding construct is a polypeptide that specifically binds to an antigen of interest. An antigen binding construct can be a monomer, dimer, multimer, a protein, a peptide, or a protein or peptide complex; an antibody, an antibody fragment, or an antigen binding fragment thereof; an scFv and the like. An antigen binding construct can be a polypeptide construct that is monospecific, bispecific, or multispecific. In some aspects, an antigen binding construct can include, e.g., one or more antigen binding components (e.g., Fabs or scFvs) linked to one or more Fc. Further examples of antigen binding constructs are described below and provided in the Examples.

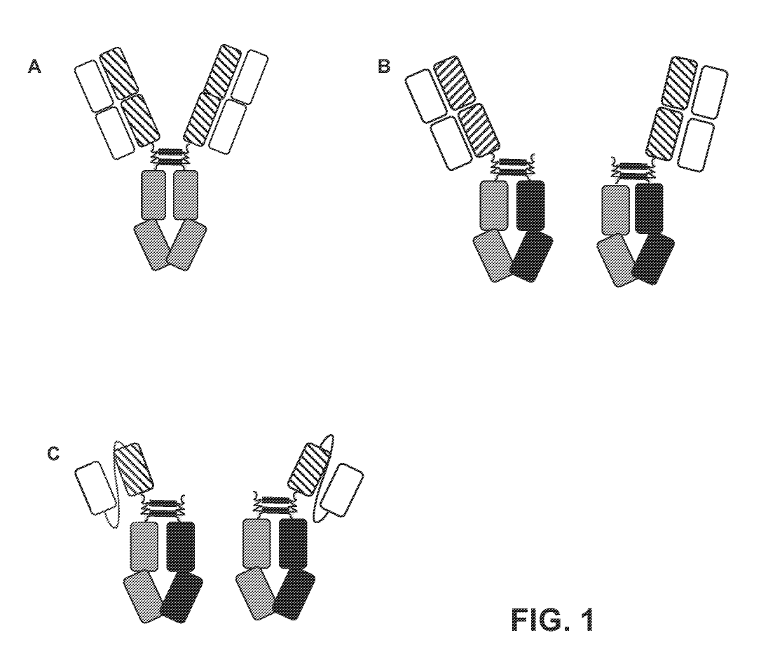

The term “monovalent antigen-binding construct” as used herein refers to an antigen-binding construct that has one antigen binding domain. The antigen binding domain could be, but is not limited to, formats such as Fab (fragment antigen binding), scFv (single chain Fv) and sdab (single domain antibody). Exemplary structures of monovalent antigen binding constructs are shown in FIGS. 1B and 1C.

The term “monospecific bivalent antigen-binding construct” as used herein refers to an antigen-binding construct which has two antigen binding domains (bivalent), both of which bind to the same epitope/antigen (monospecific). The antigen binding domains could be, but are not limited to, formats such as Fab (fragment antigen binding), scFv (single chain Fv) and sdab (single domain antibody). The monospecific bivalent antigen-binding construct is also referred to herein as a “full-size antibody” or “FSA.” An exemplary structure of a monospecific bivalent antigen-binding construct is shown in FIG. 1A. In some embodiments, a monospecific bivalent antigen-binding construct is a reference against which the properties of the monovalent antigen-binding constructs are measured. In other embodiments, a combination of two monospecific bivalent antigen-binding constructs is a reference against which the properties of a combination of two monovalent antigen-binding constructs are measured. In such cases, the reference monospecific bivalent antigen-binding construct corresponds to the monovalent antigen binding construct. For example, if the monovalent antigen-binding construct binds to an epitope in ECD2 of HER2, and the antigen-binding domain is in the Fab format, then the corresponding monospecific bivalent antigen-binding construct will also bind to the same epitope in ECD2 of HER2 and the two antigen binding domains will be also be in the Fab format. The same is true in cases where a combination of two monospecific bivalent antigen-binding constructs is used as a reference, where each of the two monospecific bivalent antigen-binding constructs will corresponds to one of the monovalent antigen-binding constructs. In some embodiments, where a combination of two monovalent antigen-binding constructs is used, a single monospecific bivalent antigen-binding construct is used as a reference, where the single monospecific bivalent antigen-binding construct represents a standard of care (SOC) therapy, for example, Herceptin™, or T-DM1.

In some embodiments, the monovalent antigen-binding construct used in the methods described herein is humanized. “Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

Humanized HER2 antibodies include huMAb4D5-1, huMAb4D5-2, huMAb4D5-3, huMAb4D5-4, huMAb4D5-5, huMAb4D5-6, huMAb4D5-7 and huMAb4D5-8 or Trastuzumab (HERCEPTIN®) as described in Table 3 of U.S. Pat. No. 5,821,337 expressly incorporated herein by reference; humanized 520C9 (WO93/21319) and 20′ humanized 2C4 antibodies as described in US Patent Publication No. 2006/0018899.

### Antigen-Binding Polypeptide Constructs

The antigen binding constructs used in the methods described herein include an antigen binding polypeptide construct, e.g., an antigen binding domain. The antigen binding polypeptide construct specifically binds to HER2. The format of the antigen binding polypeptide construct can be, e.g., a Fab format, an scFV format, or a Sdab format, depending on the application.

The “Fab fragment” format (also referred to as fragment antigen binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen binding. Fab′ fragments differ from Fab fragments by the addition of a few residues at the carboxy terminus of the heavy chain CH1 domain including one or more cysteines from the antibody hinge region.

The “Single-chain Fv” or “scFvformat includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. No. 5,571,894; and U.S. Pat. No. 5,587,458.

The “Single domain antibodies” or “Sdab” format is an individual immunoglobulin domain. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22).

The antigen-binding polypeptide construct which monovalently binds an antigen can be derived from known antibodies or antigen-binding domains, or can be derived from novel antibodies or antigen-binding domains. Selection of antigen-binding constructs is described in more detail herein.

In embodiments where the monovalent antigen-binding construct comprises an antigen-binding polypeptide construct that binds to HER2, the antigen-binding polypeptide construct can be derived from known anti-HER2 antibodies or anti-HER2 binding domains in various formats including Fab fragments, scFvs, and sdab. In certain embodiments the antigen-binding polypeptide construct can be derived from humanized, or chimeric versions of these antibodies. In one embodiment, the antigen-binding polypeptide construct is derived from a Fab fragment of trastuzumab, pertuzumab, or humanized versions thereof. Non-limiting examples of such antigen-binding polypeptide constructs include those found in monovalent antigen binding constructs described herein including but not limited to 1040, 1041, and 4182. In one embodiment, the antigen-binding polypeptide construct is derived from an scFv. Non-limiting examples of such antigen-binding polypeptide constructs include those found in the monovalent antigen-binding constructs 630 and 878. In one embodiment, the antigen-binding polypeptide construct is derived from an sdab.

As described elsewhere herein, antibodies that bind to ECD1, ECD2, or ECD4 are known in the art and include for example, 2C4 or pertuzumab (which bind ECD2), 4D5 or trastuzumab (which bind ECD4) or 7C2/F3, B1D2, or c6.5 (which bind ECD1). Other antibodies that bind HER2 have also been described in the art, for example in WO 2011/147982 (Genmab A/S). The monovalent antigen-binding constructs suitable for use in the methods of treatment described here can be derived from other known anti-HER2 antibodies that bind to ECD1, ECD2, or ECD4.

In some embodiments the antigen-binding polypeptide construct of the monovalent antigen binding construct is derived from an antibody that blocks by 50% or greater the binding of trastuzumab to ECD4 of HER2. In some embodiments, the antigen-binding polypeptide construct of the monovalent antigen binding construct is derived from an antibody that that blocks by 50% or greater the binding of pertuzumab to ECD2 of HER2. In some embodiments, the antigen-binding polypeptide construct of the monovalent antigen binding construct is derived from an antibody that blocks by 50% or greater the binding of C6.5, B1D2 or 7C2/F3 to ECD1 of HER2.

In some embodiments, the antigen binding polypeptide construct is modified to increase affinity for HER2. Examples of methods for generating and/or screening for antigen-binding constructs with increased affinity for HER2 are described herein. Non-limiting examples include those found in monovalent antigen binding constructs 4442, 4443, 4444, and 4445 described herein.

### HER2

The methods described herein include administration of at least one isolated monovalent antigen binding construct having an antigen binding polypeptide construct that that binds HER2. In some embodiments, the antigen binding polypeptide construct binds an ECD1, and ECD2, or an ECD4 of HER2.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” and “neu” refers to the gene encoding human ErbB2 protein. p185 or p185neu refers to the protein product of the neu gene. Preferred HER2 is native sequence human HER2.

The extracellular (ecto) domain of HER2 comprises four domains, Domain I (ECD1, amino acid residues from about 1-195), Domain II (ECD2, amino acid residues from about 196-319), Domain III (ECD3, amino acid residues from about 320-488), and Domain IV (ECD4, amino acid residues from about 489-630) (residue numbering without signal peptide). See Garrett et al. Mol. Cell. 11: 495-505 (2003), Cho et al. Nature 421: 756-760 (2003), Franklin et al. Cancer Cell 5:317-328 (2004), Tse et al. Cancer Treat Rev. 2012 April; 38(2):133-42 (2012), or Plowman et al. Proc. Natl. Acad. Sci. 90:1746-1750 (1993).

The sequence of HER2 is as follows; ECD boundaries are Domain I: 1-165; Domain II: 166-322; Domain III: 323-488; Domain IV: 489-607.

```
(SEQ ID NO: 318)
    1 tqvctgtdmk lrlpaspeth ldmlrhlyqg cqvvqgnlel tylptnasls flqdiqevqg
  61 yvliahnqvr qvplqrlriv rgtqlfedny alavldngdp lnnttpvtga spgglrelql
121 rslteilkgg vliqrnpqlc yqdtilwkdi fhknnqlalt lidtnrsrac hpcspmckgs
181 rcwgessedc qsltrtvcag gcarckgplp tdccheqcaa gctgpkhsdc laclhfnhsg
241 icelhcpalv tyntdtfesm pnpegrytfg ascvtacpyn ylstdvgsct lvcplhnqev
301 taedgtqrce kcskpcarvc yglgmehlre vravtsaniq efagckkifg slaflpesfd
361 gdpasntapl qpeqlqvfet leeitgylyi sawpdslpdl svfqnlqvir grilhngays
421 ltlqglgisw lglrslrelg sglalihhnt hlcfvhtvpw dqlfrnphqa llhtanrped
481 ecvgeglach qlcarghcwg pgptqcvncs qflrgqecve ecrvlqglpr eyvnarhclp
541 chpecqpqng svtcfgpead qcvacahykd ppfcvarcps gvkpdlsymp iwkfpdeega
601 cqpcpin
```

A “HER receptor” is a receptor protein tyrosine kinase which belongs to the human epidermal growth factor receptor (HER) family and includes EGFR, HER2, HER3 and HER4 receptors. The HER receptor will generally comprise an extracellular domain, which may bind an HER ligand; a lipophilic transmembrane domain; a conserved intracellular tyrosine kinase domain; and a carboxyl-terminal signaling domain harboring several tyrosine residues which can be phosphorylated.

By “HER ligand” is meant a polypeptide which binds to and/or activates an HER receptor. Examples include a native sequence human HER ligand such as epidermal growth factor (EGF) (Savage et al., J. Biol. Chem. 247:7612-7621 (1972)); transforming growth factor alpha (TGF-α) (Marquardt et al., Science 223:1079-1082 (1984)); amphiregulin also known as schwanoma or keratinocyte autocrine growth factor (Shoyab et al. Science 243:1074-1076 (1989); Kimura et al. Nature 348:257-260 (1990); and Cook et al. Mol. Cell. Biol. 11:2547-2557 (1991)); betacellulin (Shing et al., Science 259:1604-1607 (1993); and Sasada et al. Biochem. Biophys. Res. Commun. 190:1173 (1993)); heparin-binding epidermal growth factor (HB-EGF) (Higashiyama et al., Science 251:936-939 (1991)); epiregulin (Toyoda et al., J. Biol. Chem. 270:7495-7500 (1995); and Komurasaki et al. Oncogene 15:2841-2848 (1997)); a heregulin (see below); neuregulin-2 (NRG-2) (Carraway et al., Nature 387:512-516 (1997)); neuregulin-3 (NRG-3) (Zhang et al., Proc. Natl. Acad. Sci. 94:9562-9567 (1997)); neuregulin-4 (NRG-4) (Harari et al. Oncogene 18:2681-89 (1999)) or cripto (CR-1) (Kannan et al. J. Biol. Chem. 272(6):3330-3335 (1997)). HER ligands which bind EGFR include EGF, TGF-α, amphiregulin, betacellulin, HB-EGF and epiregulin. HER ligands which bind HER3 include heregulins. HER ligands capable of binding HER4 include betacellulin, epiregulin, HB-EGF, NRG-2, NRG-3, NRG-4 and heregulins.

“Heregulin” (HRG) when used herein refers to a polypeptide encoded by the heregulin gene product as disclosed in U.S. Pat. No. 5,641,869 or Marchionni et al., Nature, 362:312-318 (1993). Examples of heregulins include heregulin-α, heregulin-β1, heregulin-β2 and heregulin-β3 (Holmes et al., Science, 256:1205-1210 (1992); and U.S. Pat. No. 5,641,869); neu differentiation factor (NDF) (Peles et al. Cell 69: 205-216 (1992)); acetylcholine receptor-inducing activity (ARIA) (Falls et al. Cell 72:801-815 (1993)); glial growth factors (GGFs) (Marchionni et al., Nature, 362:312-318 (1993)); sensory and motor neuron derived factor (SMDF) (Ho et al. J. Biol. Chem. 270:14523-14532 (1995)); γ-heregulin (Schaefer et al. Oncogene 15:1385-1394 (1997)). The term includes biologically active fragments and/or amino acid sequence variants of a native sequence HRG polypeptide, such as an EGF-like domain fragment thereof (e.g. HRGβ1177-244).

“HER activation” or “HER2 activation” refers to activation, or phosphorylation, of any one or more HER receptors, or HER2 receptors. Generally, HER activation results in signal transduction (e.g. that caused by an intracellular kinase domain of a HER receptor phosphorylating tyrosine residues in the HER receptor or a substrate polypeptide). HER activation may be mediated by HER ligand binding to a HER dimer comprising the HER receptor of interest. HER ligand binding to a HER dimer may activate a kinase domain of one or more of the HER receptors in the dimer and thereby results in phosphorylation of tyrosine residues in one or more of the HER receptors and/or phosphorylation of tyrosine residues in additional substrate polypeptides(s), such as Akt or MAPK intracellular kinases.

As used herein, the term “EGFR” refers to epidermal growth factor receptor (also known as HER-1 or Erb-B1), including the human form(s) (Ulrich, A. et al., Nature 309:418-425 (1984); SwissProt Accession #P00533; secondary accession numbers: 000688, 000732, P06268, Q14225, Q92795, Q9BZS2, Q9GZX1, Q9H2C9, Q9H3C9, Q9UMD7, Q9UMD8, Q9UMG5), as well as naturally-occurring isoforms and variants thereof. Such isoforms and variants include but are not limited to the EGFRvIII variant, alternative splicing products (e.g., as identified by SwissProt Accession numbers P00533-1, P00533-2, P00533-3, P00533-4), variants GLN-98, ARG-266, Lys-521, ILE-674, GLY-962, and PRO-988 (Livingston, R. J. et al., NIEHS-SNPs, environmental genome project, NIEHS ES15478, Department of Genome Sciences, Seattle, Wash. (2004)), and others identified by the following accession numbers: NM005228.3, NM201282.1, NM201283.1, NM201284.1 (REFSEQ mRNAs); AF125253.1, AF277897.1, AF288738.1, AI217671.1, AK127817.1, AL598260.1, AU137334.1, AW163038.1, AW295229.1, BCO57802.1, CB160831.1, K03193.1, U48722.1, U95089.1, X00588.1, X00663.1; H5448451, H5448453, H5448452 (MIPS assembly); DT.453606, DT.86855651, DT.95165593, DT.97822681, DT.95165600, DT.100752430, DT.91654361, DT.92034460, DT.92446349, DT.97784849, DT.101978019, DT.418647, DT.86842167, DT.91803457, DT.92446350, DT.95153003, DT.95254161, DT.97816654, DT.87014330, DT.87079224 (DOTS Assembly). All accession numbers referenced herein are taken from the NCBI database (or other relevant, referenced database) as of Nov. 8, 2013.

In embodiments where the monovalent antigen binding construct comprises an antigen-binding polypeptide construct that binds to HER2, the antigen-binding polypeptide construct binds to HER2 or to a particular domain or epitope of HER2. In one embodiment, the antigen-binding polypeptide construct binds to an extracellular domain of HER2. As is known in the art, the HER2 antigen comprises multiple extracellular domains (ECDs).

In one embodiment is a monovalent antigen binding construct described herein which comprises an antigen-binding polypeptide construct that binds to an ECD of HER2 selected from ECD1, ECD2, ECD3, and ECD4. In another embodiment, the monovalent antigen binding construct comprises an antigen-binding polypeptide construct that binds to an ECD of HER2 selected from ECD1, ECD2, and ECD4. In one embodiment, the monovalent antigen binding construct comprises an antigen-binding polypeptide construct that binds to ECD1. In one embodiment, the monovalent antigen binding construct comprises an antigen-binding polypeptide construct that binds to ECD2. In one embodiment, the monovalent antigen binding construct comprises an antigen-binding polypeptide construct that binds to ECD4. In another embodiment, the monovalent antigen binding construct comprises an antigen-binding polypeptide construct that binds to an epitope of HER2 selected from 2C4, 4D5 and C6.5.

The “epitope 2C4” is the region in the extracellular domain of HER2 to which the antibody 2C4 binds. In order to screen for antibodies which bind to the 2C4 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 2C4 epitope of HER2 using methods known in the art and/or one can study the antibody-HER2 structure (Franklin et al. Cancer Cell 5:317-328 (2004)) to see what domain(s) of HER2 is/are bound by the antibody. Epitope 2C4 comprises residues from domain II in the extracellular domain of HER2. 2C4 and Pertuzumab bind to the extracellular domain of HER2 at the junction of domains I, II and III. Franklin et al. Cancer Cell 5:317-328 (2004).

The “epitope 4D5” is the region in the extracellular domain of HER2 to which the antibody 4D5 (ATCC CRL 10463) and Trastuzumab bind. This epitope is close to the transmembrane domain of HER2, and within Domain IV of HER2. To screen for antibodies which bind to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 4D5 epitope of HER2 (e.g. any one or more residues in the region from about residue 529 to about residue 625, inclusive, see FIG. 1 of US Patent Publication No. 2006/0018899).

The “epitope 7C2/F3” is the region at the N terminus, within Domain I, of the extracellular domain of HER2 to which the 7C2 and/or 7F3 antibodies (each deposited with the ATCC, see below) bind. To screen for antibodies which bind to the 7C2/7F3 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to establish whether the antibody binds to the 7C2/7F3 epitope on HER2 (e.g. any one or more of residues in the region from about residue 22 to about residue 53 of HER2, see FIG. 1 of US Patent Publication No. 2006/0018899).

The “epitope C6.5” is the region in domain I of the extracellular domain of HER2, to which the antibody C6.5 binds (Schier R. et al. (1995) In vitro and in vivo characterization of a human anti-c-erbB-2 single-chain Fv isolated from a filamentous phage antibody library. Immunotechnology 1, 73).

“Specifically binds”, “specific binding” or “selective binding” means that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al, Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of <1 μM, <100 nM, <10 nM, <1 nM, <0.1 nM, <0.01 nM, or <0.001 nM (e.g. 10^{˜8} M or less, e.g. from 10^{˜8} M to 10^{″13} M, e.g., from 10^{″9} M to 10^{′13} M).

### Fc

The antigen-binding constructs used in the methods described herein include an Fc, e.g., a dimeric Fc.

The term “Fc” is a C-terminal region of an immunoglobulin heavy chain that contains at least a portion of the constant region and is described in more detail below. The term includes native sequence Fc regions and variant Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md., 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence.

A “dimer” or “heterodimer” is a molecule comprising at least a first monomer polypeptide and a second monomer polypeptide. In the case of a heterodimer, one of said monomers differs from the other monomer by at least one amino acid residue. In certain embodiments, the assembly of the dimer is driven by surface area burial. In some embodiments, the monomeric polypeptides interact with each other by means of electrostatic interactions and/or salt-bridge interactions that drive dimer formation by favoring the desired dimer formation and/or disfavoring formation of other non-desired specimen. In some embodiments, the monomer polypeptides interact with each other by means of hydrophobic interactions that drive desired dimer formation by favoring desired dimer formation and/or disfavoring formation of other assembly types. In certain embodiments, the monomer polypeptides interact with each other by means of covalent bond formation. In certain embodiments, the covalent bonds are formed between naturally present or introduced cysteines that drive desired dimer formation. In certain embodiments described herein, no covalent bonds are formed between the monomers. In some embodiments, the polypeptides interact with each other by means of packing/size-complementarity/knobs-into-holes/protruberance-cavity type interactions that drive dimer formation by favoring desired dimer formation and/or disfavoring formation of other non-desired embodiments. In some embodiments, the polypeptides interact with each other by means of cation-pi interactions that drive dimer formation. In certain embodiments the individual monomer polypeptides cannot exist as isolated monomers in solution.

An Fc domain comprises either a CH3 domain or a CH3 and a CH2 domain. The CH3 domain comprises two CH3 sequences, one from each of the two Fc polypeptides of the dimeric Fc. The CH2 domain comprises two CH2 sequences, one from each of the two Fc polypeptides of the dimeric Fc.

In some aspects, the Fc comprises at least one or two CH3 sequences. In some aspects, the Fc is coupled, with or without one or more linkers, to a first antigen-binding construct and/or a second antigen-binding construct. In some aspects, the Fc is a human Fc. In some aspects, the Fc is a human IgG or IgG1 Fc. In some aspects, the Fc is a heterodimeric Fc. In some aspects, the Fc comprises at least one or two CH2 sequences.

In some aspects, the Fc comprises one or more modifications in at least one of the CH3 sequences. In some aspects, the Fc comprises one or more modifications in at least one of the CH2 sequences. In some aspects, an Fc is a single polypeptide. In some aspects, an Fc is multiple peptides, e.g., two polypeptides.

In some aspects, an Fc is an Fc described in patent applications PCT/CA2011/001238, filed Nov. 4, 2011 or PCT/CA2012/050780, filed Nov. 2, 2012, the entire disclosure of each of which is hereby incorporated by reference in its entirety for all purposes.

### Modified CH3 Domains

In some aspects, the antigen-binding construct described herein comprises a heterodimeric Fc comprising a modified CH3 domain that has been asymmetrically modified. The heterodimeric Fc can comprise two heavy chain constant domain polypeptides: a first Fc polypeptide and a second Fc polypeptide, which can be used interchangeably provided that Fc comprises one first Fc polypeptide and one second Fc polypeptide. Generally, the first Fc polypeptide comprises a first CH3 sequence and the second Fc polypeptide comprises a second CH3 sequence.

Two CH3 sequences that comprise one or more amino acid modifications introduced in an asymmetric fashion generally results in a heterodimeric Fc, rather than a homodimer, when the two CH3 sequences dimerize. As used herein, “asymmetric amino acid modifications” refers to any modification where an amino acid at a specific position on a first CH3 sequence is different from the amino acid on a second CH3 sequence at the same position, and the first and second CH3 sequence preferentially pair to form a heterodimer, rather than a homodimer. This heterodimerization can be a result of modification of only one of the two amino acids at the same respective amino acid position on each sequence; or modification of both amino acids on each sequence at the same respective position on each of the first and second CH3 sequences. The first and second CH3 sequence of a heterodimeric Fc can comprise one or more than one asymmetric amino acid modification.

Table A provides the amino acid sequence of the human IgG1 Fc sequence, corresponding to amino acids 231 to 447 of the full-length human IgG1 heavy chain. The CH3 sequence comprises amino acid 341-447 of the full-length human IgG1 heavy chain.

Typically an Fc can include two contiguous heavy chain sequences (A and B) that are capable of dimerizing. In some aspects, one or both sequences of an Fc include one or more mutations or modifications at the following locations: L351, F405, Y407, T366, K392, T394, T350, 5400, and/or N390, using EU numbering. In some aspects, an Fc includes a mutant sequence shown in Table X. In some aspects, an Fc includes the mutations of Variant 1 A-B. In some aspects, an Fc includes the mutations of Variant 2 A-B. In some aspects, an Fc includes the mutations of Variant 3 A-B. In some aspects, an Fc includes the mutations of Variant 4 A-B. In some aspects, an Fc includes the mutations of Variant 5 A-B.

TABLE A
IgG1 Fc sequences

| Human IgG1 | APELLGGPSVFLFPPKPKDTLMISRTPEVTCVVVDV | |
|---|---|---|
| Fc sequence | SHEDPEVKFNWYVDGVEVHNAKTKPREEQYNSTYRV | |
| 231-447 | VSVLTVLHQDWLNGKEYKCKVSNKALPAPIEKTISK | |
| (EU- | AKGQPREPQVYTLPPSRDELTKNQVSLTCLVKGFYP | |
| numbering) | SDIAVEWESNGQPENNYKTTPPVLDSDGSFFLYSKL | |
| | TVDKSRWQQGNVFSCSVMHEALHNHYTQKSLSLSPG | |
| | K (SEQ ID NO: 317) | |
| Variant IgG1 | | |
| Fc sequence | | |
| (231-447) | Chain | Mutations |
| 1 | A | L351Y_F405A_Y407V |
| 1 | B | T366L_K392M_T394W |
| 2 | A | L351Y_F405A_Y407V |
| 2 | B | T366L_K392L_T394W |
| 3 | A | T350V_L351Y_F405A_Y407V |
| 3 | B | T350V_T366L_K392L_T394W |
| 4 | A | T350V_L351Y_F405A_Y407V |
| 4 | B | T350V_T366L_K392M_T394W |
| 5 | A | T350V_L351Y_S400E_F405A_Y407V |
| 5 | B | T350V_T366L_N390R_K392M_T394W |

The first and second CH3 sequences can comprise amino acid mutations as described herein, with reference to amino acids 231 to 447 of the full-length human IgG1 heavy chain. In one embodiment, the heterodimeric Fc comprises a modified CH3 domain with a first CH3 sequence having amino acid modifications at positions F405 and Y407, and a second CH3 sequence having amino acid modifications at position T394. In one embodiment, the heterodimeric Fc comprises a modified CH3 domain with a first CH3 sequence having one or more amino acid modifications selected from L351Y, F405A, and Y407V, and the second CH3 sequence having one or more amino acid modifications selected from T366L, T366I, K392L, K392M, and T394W.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, and one of the first or second CH3 sequences further comprising amino acid modifications at position Q347, and the other CH3 sequence further comprising amino acid modification at position K360. In another embodiment, a heterodimeric Fc comprises a modified CH3 domain with a first CH3 sequence having amino acid modifications at positions L351, F405 and Y407, and a second CH3 sequence having amino acid modifications at position T366, K392, and T394, one of the first or second CH3 sequences further comprising amino acid modifications at position Q347, and the other CH3 sequence further comprising amino acid modification at position K360, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394 and one of said first and second CH3 sequences further comprising amino acid modification of D399R or D399K and the other CH3 sequence comprising one or more of T411E, T411D, K409E, K409D, K392E and K392D. In another embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, one of said first and second CH3 sequences further comprises amino acid modification of D399R or D399K and the other CH3 sequence comprising one or more of T411E, T411 D, K409E, K409D, K392E and K392D, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, wherein one or both of said CH3 sequences further comprise the amino acid modification of T350V.

In one embodiment, a heterodimeric Fc comprises a modified CH3 domain comprising the following amino acid modifications, where “A” represents the amino acid modifications to the first CH3 sequence, and “B” represents the amino acid modifications to the second CH3 sequence: A:L351Y_F405A_Y407V, B:T366L_K392M_T394W, A:L351Y_F405A_Y407V, B:T366L_K392L_T394W, A:T350V_L351Y_F405A_Y407V, B:T350V_T366L_K392L_T394W, A:T350V_L351Y_F405A_Y407V, B:T350V_T366L_K392M_T394W, A:T350V_L351Y_S400E_F405A_Y407V, and/or B:T350V_T366L_N390R_K392M_T394W.

The one or more asymmetric amino acid modifications can promote the formation of a heterodimeric Fc in which the heterodimeric CH3 domain has a stability that is comparable to a wild-type homodimeric CH3 domain. In an embodiment, the one or more asymmetric amino acid modifications promote the formation of a heterodimeric Fc domain in which the heterodimeric Fc domain has a stability that is comparable to a wild-type homodimeric Fc domain. In an embodiment, the one or more asymmetric amino acid modifications promote the formation of a heterodimeric Fc domain in which the heterodimeric Fc domain has a stability observed via the melting temperature (Tm) in a differential scanning calorimetry study, and where the melting temperature is within 4° C. of that observed for the corresponding symmetric wild-type homodimeric Fc domain. In some aspects, the Fc comprises one or more modifications in at least one of the C_{H3} sequences that promote the formation of a heterodimeric Fc with stability comparable to a wild-type homodimeric Fc.

In one embodiment, the stability of the CH3 domain can be assessed by measuring the melting temperature of the CH3 domain, for example by differential scanning calorimetry (DSC). Thus, in a further embodiment, the CH3 domain has a melting temperature of about 68° C. or higher. In another embodiment, the CH3 domain has a melting temperature of about 70° C. or higher. In another embodiment, the CH3 domain has a melting temperature of about 72° C. or higher. In another embodiment, the CH3 domain has a melting temperature of about 73° C. or higher. In another embodiment, the CH3 domain has a melting temperature of about 75° C. or higher. In another embodiment, the CH3 domain has a melting temperature of about 78° C. or higher. In some aspects, the dimerized CH3 sequences have a melting temperature (Tm) of about 68, 69, 70, 71, 72, 73, 74, 75, 76, 77, 77.5, 78, 79, 80, 81, 82, 83, 84, or 85° C. or higher.

In some embodiments, a heterodimeric Fc comprising modified CH3 sequences can be formed with a purity of at least about 75% as compared to homodimeric Fc in the expressed product. In another embodiment, the heterodimeric Fc is formed with a purity greater than about 80%. In another embodiment, the heterodimeric Fc is formed with a purity greater than about 85%. In another embodiment, the heterodimeric Fc is formed with a purity greater than about 90%. In another embodiment, the heterodimeric Fc is formed with a purity greater than about 95%. In another embodiment, the heterodimeric Fc is formed with a purity greater than about 97%. In some aspects, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed. In some aspects, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed via a single cell.

Additional methods for modifying monomeric Fc polypeptides to promote heterodimeric Fc formation are described in International Patent Publication No. WO 96/027011 (knobs into holes), in Gunasekaran et al. (Gunasekaran K. et al. (2010) J Biol Chem. 285, 19637-46, electrostatic design to achieve selective heterodimerization), in Davis et al. (Davis, J H. et al. (2010) Prot Eng Des Sel; 23(4): 195-202, strand exchange engineered domain (SEED) technology), and in Labrijn et al [Efficient generation of stable bispecific IgG1 by controlled Fab-arm exchange. Labrijn A F, Meesters J I, de Goeij B E, van den Bremer E T, Neijssen J, van Kampen M D, Strumane K, Verploegen S, Kundu A, Gramer M J, van Berkel P H, van de Winkel J G, Schuurman J, Parren P W. Proc Natl Acad Sci USA. 2013 Mar. 26; 110(13):5145-50.

In some embodiments an isolated antigen-binding construct described herein comprises an antigen binding polypeptide construct which binds an antigen; and a dimeric Fc that has superior biophysical properties like stability and ease of manufacture relative to an antigen binding construct which does not include the same dimeric Fc. A number of amino acid modifications in the Fc region are known in the art for selectively altering the affinity of the Fc for different Fcgamma receptors. In some aspects, the Fc comprises one or more modifications to promote selective binding of Fc-gamma receptors. These types of amino acid modifications are typically located in the CH2 domain or in the hinge region of antigen-binding construct.

### CH2 Domains

The CH2 domain of an Fc is amino acid 231-340 of the sequence shown in Table a. Exemplary mutations are listed below:
S298A/E333A/K334A, S298A/E333A/K334A/K326A (Lu Y, Vernes J M, Chiang N, et al. J Immunol Methods. 2011 Feb. 28; 365(1-2):132-41);
F243L/R292P/Y300LN305I/P396L, F243L/R292P/Y300L/L235V/P396L (Stavenhagen J B, Gorlatov S, Tuaillon N, et al. Cancer Res. 2007 Sep. 15; 67(18):8882-90; Nordstrom J L, Gorlatov S, Zhang W, et al. Breast Cancer Res. 2011 Nov. 30; 13(6):R123);
F243L (Stewart R, Thom G, Levens M, et al. Protein Eng Des Sel. 2011 September; 24(9):671-8.), S298A/E333A/K334A (Shields R L, Namenuk A K, Hong K, et al. J Biol Chem. 2001 Mar. 2; 276(9):6591-604);
S239D/I332E/A330L, S239D/I332E (Lazar G A, Dang W, Karki S, et al. Proc Natl Acad Sci USA. 2006 Mar. 14; 103(11):4005-10);
S239D/S267E, S267E/L328F (Chu S Y, Vostiar I, Karki S, et al. Mol Immunol. 2008 September; 45(15):3926-33);
S239D/D265S/S298A/I332E, S239E/S298A/K326A/A327H, G237F/S298A/A330L/I 332E, S239D/I332E/S298A, S239D/K326E/A330L/I332E/S298A, G236A/S239D/D2 70L/I332E, S239E/S267E/H268D, L234F/S267E/N325L, G237FN266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments a CH2 domain comprises one or more asymmetric amino acid modifications. In some embodiments a CH2 domain comprises one or more asymmetric amino acid modifications to promote selective binding of a Fc R. In some embodiments the CH2 domain allows for separation and purification of an isolated construct described herein.

Additional Modifications to Improve Effector Function.

In some embodiments an antigen binding construct described herein can be modified to improve its effector function. Such modifications are known in the art and include afucosylation, or engineering of the affinity of the Fc towards an activating receptor, mainly FCGR3a for ADCC, and towards C1q for CDC. The following Table B summarizes various designs reported in the literature for effector function engineering.

Thus, in one embodiment, a construct described herein can include a dimeric Fc that comprises one or more amino acid modifications as noted in Table B that confer improved effector function. In another embodiment, the construct can be afucosylated to improve effector function.

TABLE B
CH2 domains and effector function engineering.

| Reference | Mutations | Effect |
|---|---|---|
| Lu, 2011, | Afucosylated | Increased ADCC |
| Ferrara 2011, | | |
| Mizushima 2011 | | |
| Lu, 2011 | S298A/E333A/K334A | Increased ADCC |
| Lu, 2011 | S298A/E333A/K334A/K326A | Increased ADCC |
| Stavenhagen, 2007 | F243L/R292P/Y300L/V305I/ | Increased ADCC |
| | P396L | |
| Nordstrom, 2011 | F243L/R292P/Y300L/L235V/ | Increased ADCC |
| | P396L | |
| Stewart, 2011 | F243L | Increased ADCC |
| Shields, 2001 | S298A/E333A/K334A | Increased ADCC |
| Lazar, 2006 | S239D/I332E/A330L | Increased ADCC |
| Lazar, 2006 | S239D/I332E | Increased ADCC |
| Bowles, 2006 | AME-D, not specified | Increased ADCC |
| | mutations | |
| Heider, 2011 | 37.1, mutations not | Increased ADCC |
| | disclosed | |
| Moore, 2010 | S267E/H268F/S324T | Increased CDC |

Fc modifications reducing FcγR and/or complement binding and/or effector function are known in the art. Recent publications describe strategies that have been used to engineer antibodies with reduced or silenced effector activity (see Strohl, W R (2009), Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, US Patent Publication No. 2011/0212087 (Strohl), International Patent Publication No. WO 2006/105338 (Xencor), US Patent Publication No. 2012/0225058 (Xencor), US Patent Publication No. 2012/0251531 (Genentech), and Strop et al ((2012) J. Mol. Biol. 420: 204-219) describe specific modifications to reduce FcγR or complement binding to the Fc.

Specific, non-limiting examples of known amino acid modifications include those identified in the following table:

TABLE C
modifications to reduce FcγR or complement binding to the Fc

| Company | Mutations |
|---|---|
| GSK | N297A |
| Ortho Biotech | L234A/L235A |
| Protein Design labs | IGG2 V234A/G237A |
| Wellcome Labs | IGG4 L235A/G237A/E318A |
| GSK | IGG4 S228P/L236E |
| Alexion | IGG2/IGG4combo |
| Merck | IGG2 H268Q/V309L/A330S/A331S |
| Bristol-Myers | C220S/C226S/C229S/P238S |
| Seattle Genetics | C226S/C229S/E3233P/L235V/L235A |
| Amgen | E. coli production, non glyco |
| Medimune | L234F/L235E/P331S |
| Trubion | Hinge mutant, possibly C226S/P230S |

In one embodiment, the Fc comprises at least one amino acid modification identified in the above table. In another embodiment the Fc comprises amino acid modification of at least one of L234, L235, or D265. In another embodiment, the Fc comprises amino acid modification at L234, L235 and D265. In another embodiment, the Fc comprises the amino acid modification L234A, L235A and D265S.

### FcRn Binding and PK Parameters

As is known in the art, binding to FcRn recycles endocytosed antibody from the endosome back to the bloodstream (Raghavan et al., 1996, Annu Rev Cell Dev Biol 12:181-220; Ghetie et al., 2000, Annu Rev Immunol 18:739-766). This process, coupled with preclusion of kidney filtration due to the large size of the full-length molecule, results in favorable antibody serum half-lives ranging from one to three weeks. Binding of Fc to FcRn also plays a key role in antibody transport. Thus, in one embodiment, the antigen-binding constructs of the described herein are able to bind FcRn.

### Linkers

The antigen-binding constructs described herein can include one or more antigen binding polypeptide constructs operatively coupled to an Fc described herein. In some aspects, an Fc is coupled to the one or more antigen binding polypeptide constructs with one or more linkers. In some aspects, Fc is directly coupled to the one or more antigen binding polypeptide constructs. In some aspects, Fc is coupled to the heavy chain of each antigen binding polypeptide by a linker.

In some aspects, the one or more linkers are one or more polypeptide linkers. In some aspects, the one or more linkers comprise one or more IgG1 hinge regions.

### Selection of Antigen-Binding Constructs

The antigen-binding construct used in the methods described herein can be selected for use using any number of assays well-known to one of skill in the art.

Affinity Maturation

In instances where it is desirable to increase the affinity of the antigen-binding polypeptide construct for its cognate antigen, methods known in the art can be used to increase the affinity of the antigen-binding polypeptide construct for its antigen. Examples of such methods are described in the following references, Birtalan et al. (2008) JMB 377, 1518-1528; Gerstner et al. (2002) JMB 321, 851-862; Kelley et al. (1993) Biochem 32(27), 6828-6835; Li et al. (2010) JBC 285(6), 3865-3871, and Vajdos et al. (2002) JMB 320, 415-428.

One example, of such a method is affinity maturation. One exemplary method for affinity maturation of HER2 antigen-binding domains is described as follows. Structures of the trastuzumab/HER2 (PDB code 1N8Z) complex and pertuzumab/HER2 complex (PDB code 1S78) are used for modeling. Molecular dynamics (MD) can be employed to evaluate the intrinsic dynamic nature of the WT complex in an aqueous environment. Mean field and dead-end elimination methods along with flexible backbones can be used to optimize and prepare model structures for the mutants to be screened. Following packing a number of features will be scored including contact density, clash score, hydrophobicity and electrostatics. Generalized Born method will allow accurate modeling of the effect of solvent environment and compute the free energy differences following mutation of specific positions in the protein to alternate residue types. Contact density and clash score will provide a measure of complementarity, a critical aspect of effective protein packing. The screening procedure employs knowledge-based potentials as well as coupling analysis schemes relying on pair-wise residue interaction energy and entropy computations. Literature mutations known to enhance HER2 binding are summarized in the following tables:

TABLE A4
Trastuzumab mutations known to increase binding
to HER2 for the Trastuzumab-HER2 system.

| Mutation | Reported Improvement |
|---|---|
| H_D102W (H_D98W) | 3.2X |
| H_D102Y | 3.1X |
| H_D102K | 2.3X |
| H_D102T | 2.2X |
| H_N55K | 2.0X |
| H_N55T | 1.9X |
| L_H91F | 2.1X |
| L_D28R | 1.9X |

TABLE A5
Pertuzumab mutations known to increase binding
to HER2 for the Pertuzumab-HER2 system.

| Mutation | Reported Improvement |
|---|---|
| L_I31A | 1.9X |
| L_Y96A | 2.1X |
| L_Y96F | 2.5X |
| H_T30A | 2.1X |
| H_G56A | 8.3X |
| H_F63V | 1.9X |

Suitable monovalent antigen-binding constructs possess properties such as i) increased maximal binding (Bmax) at saturating antibody concentration to a HER2+ cancer cell; ii) the ability to be internalized in a HER2+ cancer cell; iii) the ability to mediate effector cell functions resulting in HER2+ cancer cell cytotoxicity, and/or the ability to inhibit the growth of HER2+ cancer cells.

The monovalent antigen-binding constructs described herein are internalized once they bind to the target cell. In one embodiment, the monovalent antigen-binding constructs are internalized to a similar degree compared to the corresponding monospecific bivalent antigen-binding constructs. In some embodiments, the monovalent antigen-binding constructs are internalized more efficiently compared to the corresponding monospecific bivalent antigen-binding constructs.

### Target Cells

The target cell is selected based on the intended use of the monovalent antigen-binding construct. In one embodiment, the target cell is a cell which is activated or amplified in a cancer, an infectious disease, an autoimmune disease, or in an inflammatory disease.

In one embodiment, where the monovalent antigen-binding construct is intended for use in the treatment of cancer, the target cell is derived from a tumor that exhibits EGFR and/or HER2 3+ overexpression, e.g., SKBR3 and BT474. In one embodiment, the target cell is derived from a tumor that exhibits EGFR and/or HER2 low expression, e.g., MCF7. In one embodiment, the target cell is derived from a tumor that exhibits EGFR and/or HER2 resistance, e.g., JIMT1. In one embodiment, the target cell is derived from a tumor that is a triple negative (ER/PR/HER2) tumor.

In embodiments where the monovalent antigen-binding construct is intended for use in the treatment of cancer, the target cell is a cancer cell line that is representative of EGFR and/or HER2 3+ overexpression. In one embodiment, the target cell is a cancer cell line that is representative of EGFR and/or HER2 low expression. In one embodiment, the target cell is a cancer cell line that is representative of EGFR and/or HER2 resistance. In one embodiment, the target cell is a cancer cell line that is representative of breast cancer triple negative e.g., MDA-MD-231 cells.

In one embodiment, the monovalent antigen-binding construct described herein is designed to target a breast cancer cell or epithelial cell-derived cancer cell. Examples include but are not limited to the following: progesterone receptor (PR) negative and estrogen receptor (ER) negative cells, low HER 2 expressing cells, medium HER-2 expressing cells, high HER2 expressing cells, anti-HER2 antibody resistant cells, or epithelial cell-derived cancer cells.

In one embodiment, the monovalent antigen-binding construct described herein is designed to target Gastric and Esophageal Adenocarcinomas. Exemplary histologic types include: HER2 positive proximal gastric carcinomas with intestinal phenotype and HER2 positive distal diffuse gastric carcinomas. Exemplary classes of gastric cancer cells include but are not limited to (N-87, OE-19, SNU-216 and MKN-7).

In another embodiment, a monovalent antigen-binding construct described herein is designed to target Metastatic HER2+ Breast Cancer Tumors in the Brain. Exemplary classes of gastric cancer cells include but are not limited to BT474.

In embodiments where the monovalent antibody construct comprises an antigen-binding polypeptide construct that binds to HER2, the antigen-binding polypeptide construct binds to HER2 or to a particular domain or epitope of HER2. In one embodiment, the antigen-binding polypeptide construct binds to an extracellular domain of HER2. As is known in the art, the HER2 antigen comprises multiple extracellular domains (ECDs).

In one embodiment is a monovalent antibody construct described herein which comprises an antigen-binding polypeptide construct that binds to an ECD of HER2 selected from ECD1, ECD2, ECD3, and ECD4. In another embodiment, the monovalent antibody construct comprises an antigen-binding polypeptide construct that binds to an ECD of HER2 selected from ECD1, ECD2, and ECD4. In one embodiment, the monovalent antibody construct comprises an antigen-binding polypeptide construct that binds to ECD1. In one embodiment, the monovalent antibody construct comprises an antigen-binding polypeptide construct that binds to ECD2. In one embodiment, the monovalent antibody construct comprises an antigen-binding polypeptide construct that binds to ECD4. In another embodiment, the monovalent antibody construct comprises an antigen-binding polypeptide construct that binds to an epitope of HER2 selected from 2C4, 4D5 and C6.5.

### Dissociation Constant (K_{D}) and Maximal Binding (Bmax)

In some embodiments, an antigen binding construct is described by functional characteristics including but not limited to a dissociation constant and a maximal binding.

The term “dissociation constant (KD)” as used herein, is intended to refer to the equilibrium dissociation constant of a particular ligand-protein interaction. As used herein, ligand-protein interactions refer to, but are not limited to protein-protein interactions or antibody-antigen interactions. The KD measures the propensity of two proteins (e.g. AB) to dissociate reversibly into smaller components (A+B), and is define as the ratio of the rate of dissociation, also called the “off-rate (k_{off})”, to the association rate, or “on-rate (kₒₙ)”. Thus, KD equals k_{off}/kₒₙ and is expressed as a molar concentration (M). It follows that the smaller the KD, the stronger the affinity of binding. Therefore, a KD of 1 mM indicates weak binding affinity compared to a KD of 1 nM. KD values for antigen binding constructs can be determined using methods well established in the art. One method for determining the KD of an antigen binding construct is by using surface plasmon resonance (SPR), typically using a biosensor system such as a Biacore® system. Isothermal titration calorimetry (ITC) is another method that can be used to determine.

The binding characteristics of an antigen binding construct can be determined by various techniques. One of which is the measurement of binding to target cells expressing the antigen by flow cytometry (FACS, Fluorescence-activated cell sorting). Typically, in such an experiment, the target cells expressing the antigen of interest are incubated with antigen binding constructs at different concentrations, washed, incubated with a secondary agent for detecting the antigen binding construct, washed, and analyzed in the flow cytometer to measure the median fluorescent intensity (MFI) representing the strength of detection signal on the cells, which in turn is related to the number of antigen binding constructs bound to the cells. The antigen binding construct concentration vs. MFI data is then fitted into a saturation binding equation to yield two key binding parameters, Bmax and apparent KD.

Apparent KD, or apparent equilibrium dissociation constant, represents the antigen binding construct concentration at which half maximal cell binding is observed. Evidently, the smaller the KD value, the smaller antigen binding construct concentration is required to reach maximum cell binding and thus the higher is the affinity of the antigen binding construct. The apparent KD is dependent on the conditions of the cell binding experiment, such as different receptor levels expressed on the cells and incubation conditions, and thus the apparent KD is generally different from the KD values determined from cell-free molecular experiments such as SPR and ITC. However, there is generally good agreement between the different methods.

The term “Bmax”, or maximal binding, refers to the maximum antigen binding construct binding level on the cells at saturating concentrations of antigen binding construct. This parameter can be reported in the arbitrary unit MFI for relative comparison, or converted into an absolute value corresponding to the number of antigen binding constructs bound to the cell with the use of a standard curve. In some embodiments, the antigen binding constructs display a Bmax that is 1.1, 1.2, 1.3, 1.4, 1.5, 1.6, 1.7, 1.8, 1.9, or 2.0 times the Bmax of a reference antigen binding construct.

For the antigen binding constructs described herein, the clearest separation in Bmax versus FSA occurs at saturating concentrations and where Bmax can no longer be increased with a FSA. The significance is less at non-saturating concentrations. In one embodiment the increase in Bmax and KD of the antigen binding construct compared to a reference antigen binding construct is independent of the level of target antigen expression on the target cell. In one embodiment, the monovalent antigen binding construct exhibits a 1.1 to 1.5-fold increase in Bmax compared to the corresponding bivalent antigen binding construct in a target cell. In one embodiment, a combination of monovalent antigen binding constructs exhibits a 1.1 to 1.5-fold increase in Bmax compared to the combination of the corresponding bivalent antigen binding constructs in a target cell.

In some embodiments is an isolated antigen binding construct described herein, wherein said antigen binding construct displays an increase in Bmax (maximum binding) to a target cell displaying said antigen as compared to a corresponding reference antigen binding construct. In some embodiments said increase in Bmax is at least about 125% of the Bmax of the corresponding reference antigen binding construct. In certain embodiments, the increase in Bmax is at least about 150% of the Bmax of the corresponding reference antigen binding construct. In some embodiments, the increase in Bmax is at least about 200% of the Bmax of the corresponding reference antigen binding construct. In some embodiments, the increase in Bmax is greater than about 110% of the Bmax of the corresponding reference antigen binding construct.

### Efficacy/Bioactivity

As indicated herein, the monovalent antigen-binding constructs described herein display superior efficacy and/or bioactivity as compared to the corresponding monospecific bivalent antigen-binding construct. Non-limiting examples of the efficacy and/or bioactivity of the monovalent antigen-binding constructs described herein are represented by the ability of the monovalent antigen-binding construct to inhibit growth of the target cell or mediate effector cell-mediated cell killing. In one embodiment, the superior efficacy and/or bioactivity of the monovalent antigen-binding constructs is mainly a result of increased effector function of the monovalent antigen-binding construct compared to the monospecific bivalent antigen-binding construct.

Antibody “effector functions” refer to those biological activities attributable to the Fc domain (a native sequence Fc domain or amino acid sequence variant Fc domain) of an antibody. Examples of antibody effector functions include antibody dependent cellular phagocytosis (ADCP), C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell.

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen.

“Antibody-dependent cellular phagocytosis and “ADCP” refer to the destruction of target cells via monocyte or macrophage-mediated phagocytosis.

The terms “Fc receptor” and “FcR” are used to describe a receptor that binds to the Fc domain of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu Rev. Immunol. 15:203-234 (1997)). FcRs are reviewed in Ravetch and Kinet, Annu Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

The term “avidity” is used here to refer to the combined synergistic strength of binding affinities and a key structure and biological attribute of therapeutic monospecific bivalent antibodies. Lack of avidity and loss of synergistic strength of binding can result in reduced apparent target binding affinity. On the other hand, on a target cell with fixed number of antigens, avidity resulting from the multivalent (or bivalent) binding causes increased occupancy of the target antigen at a lower number of antibody molecules relative to antibody which displays monovalent binding. With a lower number of antibody molecules bound to the target cell, in the application of bivalent lytic antibodies, antibody dependent cytotoxic killing mechanisms may not occur efficiently resulting in reduced efficacy. Not enough antibodies are bound to mediate ADCC, CDC, ADCP as these types of effector functions are generally considered to be Fc concentration threshold dependent. In the case of agonistic antibodies, reduced avidity reduces their efficiency to crosslink and dimerize antigens and activate the pathway.

ADCC

Thus, in one embodiment, the monovalent antigen-binding construct exhibits a higher degree of cell killing by ADCC than does the corresponding monospecific bivalent antigen-binding construct. In accordance with this embodiment, the monovalent antigen-binding construct exhibits an increase in ADCC activity of between about 1.2- to 1.8-fold over that of the corresponding monospecific bivalent antigen-binding construct. In one embodiment, the monovalent antigen-binding construct exhibits about a 1.3-fold increase in cell killing by ADCC than does the corresponding monospecific bivalent antigen-binding construct. In one embodiment, the monovalent antigen-binding construct exhibits about a 1.4-fold increase in cell killing by ADCC than does the corresponding monospecific bivalent antigen-binding construct. In one embodiment, the monovalent antigen-binding construct exhibits about a 1.5-fold increase in cell killing by ADCC than does the corresponding monospecific bivalent antigen-binding construct.

In one embodiment, the monovalent antigen-binding construct comprises an antigen-binding polypeptide construct that binds to EGFR and/or HER2 and exhibits an increase in ADCC activity of between about 1.2- to 1.6-fold over that of the corresponding monospecific bivalent antigen-binding construct. In one embodiment, the monovalent antigen-binding construct comprises an antigen-binding polypeptide construct that binds to EGFR and/or HER2 and exhibits about a 1.3-fold increase in cell killing by ADCC than does the corresponding monospecific bivalent antigen-binding construct. In one embodiment, the monovalent antigen-binding construct comprises an antigen-binding polypeptide construct that binds to EGFR and/or HER2 and exhibits about a 1.5-fold increase in cell killing by ADCC than does the corresponding monospecific bivalent antigen-binding construct.

ADCP

In one embodiment, the monovalent antigen-binding construct exhibits a higher degree of cell killing by ADCP than does the corresponding monospecific bivalent antigen-binding construct.

CDC

In one embodiment, the monovalent antigen-binding construct exhibits a higher degree of cell killing by CDC than does the corresponding monospecific bivalent antigen-binding construct. In one embodiment, the monovalent antigen-binding construct comprises an antigen-binding polypeptide construct that binds to EGFR and/or HER2 and exhibits about a 1.5-fold increase in cell killing by CDC than does the corresponding monospecific bivalent antigen-binding construct.

In some embodiments is an isolated monovalent antigen-binding construct described herein, wherein said construct possesses at least about 125% of at least one of the ADCC, ADCP and CDC of a corresponding bivalent antigen-binding construct with two antigen binding polypeptide constructs. In some embodiments is an isolated monovalent antigen-binding construct described herein, wherein said construct possesses at least about 150% of at least one of the ADCC, ADCP and CDC of a corresponding bivalent antigen-binding construct with two antigen binding polypeptide constructs. In some embodiments is an isolated monovalent antigen-binding construct described herein, wherein said construct possesses at least about 300% of at least one of the ADCC, ADCP and CDC of a corresponding bivalent antigen-binding construct with two antigen binding polypeptide constructs.

Increased Binding Capacity to FcγRs

In some embodiments, the monovalent antigen-binding constructs exhibit a higher binding capacity (Rmax) to one or more FcγRs. In one embodiment where the monovalent antigen-binding construct comprises an antigen-binding polypeptide construct that binds to HER2, the monovalent antigen-binding construct exhibits an increase in Rmax to one or more FcγRs over the corresponding monospecific bivalent antigen-binding construct of between about 1.3- to 2-fold. In one embodiment where the monovalent antigen-binding construct comprises an antigen-binding polypeptide construct that binds to EGFR and/or HER2, the monovalent antigen-binding construct exhibits an increase in Rmax to a CD16 FcγR of between about 1.3- to 1.8-fold over the corresponding monospecific bivalent antigen-binding construct. In one embodiment where the monovalent antigen-binding construct comprises an antigen-binding polypeptide construct that binds to EGFR and/or HER2, the monovalent antigen-binding construct exhibits an increase in Rmax to a CD32 FcγR of between about 1.3- to 1.8-fold over the corresponding monospecific bivalent antigen-binding construct. In one embodiment where the monovalent antigen-binding construct comprises an antigen-binding polypeptide construct that binds to EGFR and/or HER2, the monovalent antigen-binding construct exhibits an increase in Rmax to a CD64 FcγR of between about 1.3- to 1.8-fold over the corresponding monospecific bivalent antigen-binding construct.

Increased Affinity for FcγRs

In one embodiment, the monovalent antigen-binding constructs provided herein have an unexpectedly increased affinity for FcγR as compared to corresponding bivalent antigen-binding constructs. The increased Fc concentration resulting from the binding is consistent with increased ADCC, ADCP, CDC activity.

In some embodiments, the monovalent antigen-binding constructs exhibit an increased affinity for one or more FcγRs. In one embodiment, where the monovalent antigen-binding construct comprises an antigen-binding polypeptide construct that binds to HER2, the monovalent antigen-binding constructs exhibit an increased affinity for at least one FcγR. In accordance with this embodiment, the monovalent antigen-binding construct exhibits an increased affinity for CD32.

In another embodiment, is a monovalent antigen-binding construct described herein that exhibits increased internalization compared to a corresponding monospecific bivalent antigen-binding construct, thereby resulting in superior efficacy and/or bioactivity.

Testing of the Monovalent Antigen-Binding Constructs. FcγR, FcRn and C1q Binding

The monovalent antigen-binding constructs described herein exhibit enhanced effector function compared to the corresponding monospecific bivalent antigen-binding construct. The effector functions of the monovalent antigen-binding constructs can be tested as follows. In vitro and/or in vivo cytotoxicity assays can be conducted to assess ADCP, CDC and/or ADCC activities. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu Rev. Immunol 9:457-92 (1991). An example of an in vitro assay to assess ADCC activity of a molecule of interest is described in U.S. Pat. No. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). C1q binding assays may also be carried out to determine if the monovalent antigen-binding constructs are capable of binding C1q and hence activating CDC. To assess complement activation, a CDC assay, e.g. as described in Gazzano-Santoro et al., J. Immunol. Methods 202:163 (1996), may be performed. FcRn binding such as by SPR and in vivo PK determinations of antibodies can also be performed using methods well known in the art.

Pharmacokinetic Parameters

In certain embodiments, a monovalent antigen-binding construct provided herein exhibits pharmacokinetic (PK) properties comparable with commercially available therapeutic antibodies. In one embodiment, the monovalent antigen-binding constructs described herein exhibit PK properties similar to known therapeutic antibodies, with respect to serum concentration, t1/2, beta half-life, and/or CL. In one embodiment, the monovalent antigen-binding constructs display in vivo stability comparable ro or greater than said monospecific bivalent antigen-binding construct. Such in vivo stability parameters include serum concentration, t1/2, beta half-life, and/or C_{L}.

In one embodiment, the monovalent antigen-binding constructs provided herein show a higher volume of distribution (Vss) compared to the corresponding monospecific bivalent antigen-binding constructs. Volume of distribution of an antibody relates to volume of plasma or blood (Vp), the volume of tissue (VT), and the tissue-to-plasma partitioning (kP). Under linear conditions, IgG antibodies are primarily distributed into the plasma compartment and the extravascular fluid following intravascular administration in animals or humans. In some embodiments, active transport processes such as uptake by neonatal Fc receptor (FcRn) also impact antibody biodistribution among other binding proteins.

In another embodiment, the monovalent antigen-binding constructs described herein show a higher volume of distribution (Vss) and bind FcRn with similar affinity compared to the corresponding monospecific bivalent antigen-binding constructs.

As used herein, the term “modulated serum half-life” means the positive or negative change in circulating half-life of an antigen binding polypeptide that is comprised by an antigen-binding construct described herein relative to its native form. Serum half-life is measured by taking blood samples at various time points after administration of the construct, and determining the concentration of that molecule in each sample. Correlation of the serum concentration with time allows calculation of the serum half-life. Increased serum half-life desirably has at least about two-fold, but a smaller increase may be useful, for example where it enables a satisfactory dosing regimen or avoids a toxic effect. In some embodiments, the increase is at least about three-fold, at least about five-fold, or at least about ten-fold.

The term “modulated therapeutic half-life” as used herein means the positive or negative change in the half-life of the therapeutically effective amount of an antigen binding polypeptide comprised by a monovalent antigen-binding construct described herein, relative to its non-modified form. Therapeutic half-life is measured by measuring pharmacokinetic and/or pharmacodynamic properties of the molecule at various time points after administration. Increased therapeutic half-life desirably enables a particular beneficial dosing regimen, a particular beneficial total dose, or avoids an undesired effect. In some embodiments, the increased therapeutic half-life results from increased potency, increased or decreased binding of the modified molecule to its target, increased or decreased breakdown of the molecule by enzymes such as proteases, or an increase or decrease in another parameter or mechanism of action of the non-modified molecule or an increase or decrease in receptor-mediated clearance of the molecule.

### Production of Antigen-Binding Constructs

Antigen-binding constructs may be produced using recombinant methods and compositions, e.g., as described in U.S. Pat. No. 4,816,567. In one embodiment, isolated nucleic acid encoding an antigen-binding construct described herein is provided. Such nucleic acid may encode an amino acid sequence comprising the VL and/or an amino acid sequence comprising the VH of the antigen-binding construct (e.g., the light and/or heavy chains of the antigen-binding construct). In a further embodiment, one or more vectors (e.g., expression vectors) comprising such nucleic acid are provided. In one embodiment, the nucleic acid is provided in a multicistronic vector. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antigen-binding construct and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g. a Chinese Hamster Ovary (CHO) cell, or human embryonic kidney (HEK) cell, or lymphoid cell (e.g., Y0, NS0, Sp20 cell). In one embodiment, a method of making an antigen-binding construct is provided, wherein the method comprises culturing a host cell comprising nucleic acid encoding the antigen-binding construct, as provided above, under conditions suitable for expression of the antigen-binding construct, and optionally recovering the antigen-binding construct from the host cell (or host cell culture medium).

For recombinant production of the antigen-binding construct, nucleic acid encoding an antigen-binding construct, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antigen-binding construct).

In some embodiments, the expressed antigen-binding construct includes a signal peptides. Examples include but are not limited to a Stanniocalcin signal sequence (SEQ ID NO:1) and a consensus signal sequence (SEQ ID NO:2).

Suitable host cells for cloning or expression of antigen-binding construct-encoding vectors include prokaryotic or eukaryotic cells described herein. For example, antigen-binding construct may be produced in bacteria, in particular when glycosylation and Fc effector function are not needed. For expression of antigen-binding construct fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antigen-binding construct may be isolated from the bacterial cell paste in a soluble fraction and can be further purified.

In addition to prokaryotes, eukaryotic microbes such as filamentous fungi or yeast are suitable cloning or expression hosts for antigen-binding construct-encoding vectors, including fungi and yeast strains whose glycosylation pathways have been “humanized,” resulting in the production of an antigen-binding construct with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antigen-binding constructs are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antigen-binding constructs in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antigen-binding construct production, see, e.g., Yazaki and Wu, Methods in Molecular Biology, Vol. 248 (B. K. C. Lo, ed., Humana Press, Totowa, N.J.), pp. 255-268 (2003).

In one embodiment, the antigen-binding constructs described herein are produced in stable mammalian cells, by a method comprising: transfecting at least one stable mammalian cell with: nucleic acid encoding the antigen-binding construct, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antigen-binding construct in the expressed product.

In some embodiments is the method of producing a monovalent antigen-binding construct in stable mammalian cells as described herein wherein the expression product of the at least one stable mammalian cell comprises a larger percentage of the desired glycosylated monovalent antibody as compared to the monomeric heavy or light chain polypeptides, or other antibodies.

In some embodiments is the method of producing a glycosylated monovalent antigen-binding construct in stable mammalian cells described herein, said method comprising identifying and purifying the desired glycosylated monovalent antibody. In some embodiments, the said identification is by one or both of liquid chromatography and mass spectrometry.

If required, the antigen-binding constructs can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antigen-binding constructs. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed, or immobilized anti-flag antibody if a flag-tag is used. For general guidance in suitable purification techniques, see, e.g. incorporated entirely by reference Protein Purification: Principles and Practice, 3^{rd} Ed., Scopes, Springer-Verlag, NY, 1994, incorporated entirely by reference. The degree of purification necessary will vary depending on the use of the antigen-binding constructs. In some instances no purification is necessary.

In certain embodiments the antigen-binding constructs are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antigen-binding constructs described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, -alanine, fluoro-amino acids, designer amino acids such as -methyl amino acids, C-methyl amino acids, N-methyl amino acids, and amino acid analogs in general. Furthermore, the amino acid can be D (dextrorotary) or L (levorotary).

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

As used herein, the term “medium” or “media” includes any culture medium, solution, solid, semi-solid, or rigid support that may support or contain any host cell, including bacterial host cells, yeast host cells, insect host cells, plant host cells, eukaryotic host cells, mammalian host cells, CHO cells, prokaryotic host cells, E. coli, or Pseudomonas host cells, and cell contents. Thus, the term may encompass medium in which the host cell has been grown, e.g., medium into which the protein has been secreted, including medium either before or after a proliferation step. The term also may encompass buffers or reagents that contain host cell lysates, such as in the case where an antigen-binding construct described herein is produced intracellularly and the host cells are lysed or disrupted to release the heteromultimer.

### Testing of Antigen Binding Constructs

The antigen binding constructs or pharmaceutical compositions described herein are tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific antigen-binding construct is indicated, include in vitro cell culture assays, or in vitro assays in which a patient tissue sample is grown in culture, and exposed to or otherwise administered antigen binding construct, and the effect of such antigen binding construct upon the tissue sample is observed.

Candidate monovalent antigen binding constructs can be assayed using cells, e.g., breast cancer cell lines, expressing HER2. The following Table A describes the expression level of HER2 on several representative cancer cell lines.

TABLE A5
Relative expression levels of HER2 in cell lines of interest.

| | | | |
|---|---|---|---|
| WI-38 | Normal lung | 0 | 1.0 × 10E4 |
| MDA-MB- | Triple negative | 0/1+ | 1.7 × 10E4-2.3 × 10E4 |
| 231 | breast | | |
| MCF-7 | Estrogen receptor | 1+ | 4 × 10E4-7 × 10E |
| | positive breast | | |
| JIMT-1 | Trastuzumab | 2+ | 2 × 10E5-8 × 10E5 |
| | resistant breast | | |
| ZR-75-1 | Estrogen receptor | 2+ | 3 × 10E5 |
| | positive breast | | |
| SKOV3 | ovarian | 2/3+ | 5 × 10E5-1 × 10E6 |
| SKBr3 | breast | 3+ | >1 × 10E6 |
| BT-474 | breast | 3+ | >1 × 10E6 |
| Cell | Description | IHC | HER2 receptors/cell |
| Line | | scoring | |

McDonagh et al Mol Cancer Ther. 2012 March; 11(3):582-93
Subik et al. (2010) Breast Cancer: Basic Clinical Research:4; 35-41
Carter et al. PNAS, 1994:89; 4285-4289; Yarden 2000, HER2: Basic Research, Prognosis and Therapy
Hendricks et al Mol Cancer Ther 2013; 12:1816-28

As is known in the art, a number of assays may employed in order to identify monovalent antigen-binding constructs suitable for use in the methods described herein. These assays can be carried out in cancer cells expressing HER2. Examples of suitable cancer cells are identified in Table A5. Examples of assays that may be carried out are described as follows.

For example, to identify growth inhibitory candidate monovalent antigen-binding constructs that bind HER2, one may screen for antibodies which inhibit the growth of cancer cells which express HER2. In one embodiment, the candidate antigen-binding construct of choice is able to inhibit growth of cancer cells in cell culture by about 20-100% and preferably by about 50-100% at compared to a control antigen-binding construct.

To select for candidate antigen-binding constructs which induce cell death, loss of membrane integrity as indicated by, e.g., PI (phosphatidylinositol), trypan blue or 7AAD uptake may be assessed relative to control.

In order to select for candidate antigen-binding constructs which induce apoptosis, an annexin binding assay may be employed. In addition to the annexin binding assay, a DNA staining assay may also be used.

In one embodiment, the candidate monovalent antigen-binding construct of interest may block heregulin dependent association of ErbB2 with ErbB3 in both MCF7 and SK-BR-3 cells as determined in a co-immunoprecipitation experiment substantially more effectively than monoclonal antibody 4D5, and preferably substantially more effectively than monoclonal antibody 7F3.

To screen for monovalent antigen-binding constructs which bind to an epitope on ErbB2 bound by an antibody of interest, a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

The results obtained in the cell-based assays described above can then be followed by testing in animal, e.g. murine, models, and human clinical trials.

### Antigen Binding Constructs and Antibody Drug Conjugates (ADC)

In certain embodiments an antigen binding construct is conjugated to a drug, e.g., a toxin, a chemotherapeutic agent, an immune modulator, or a radioisotope. Several methods of preparing ADCs (antibody drug conjugates or antigen binding construct drug conjugates) are known in the art and are described in U.S. Pat. No. 8,624,003 (pot method), U.S. Pat. No. 8,163,888 (one-step), and U.S. Pat. No. 5,208,020 (two-step method) for example.

In some embodiments, the drug is selected from a maytansine, auristatin, calicheamicin, or derivative thereof. In other embodiments, the drug is a maytansine selected from DM1 and DM4. Further examples are described below.

In some embodiments the drug is conjugated to the isolated antigen binding construct with an SMCC linker (DM1), or an SPDB linker (DM4). Additional examples are described below. The drug-to-antigen binding protein ratio (DAR) can be, e.g., 1.0 to 6.0 or 3.0 to 5.0 or 3.5-4.2.

In some embodiments the antigen binding construct is conjugated to a cytotoxic agent. The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32, and Lu177), chemotherapeutic agents, and toxins such as small molecule toxins or enzymatically active toxins of bacterial, fungal, plant or animal origin, including fragments and/or variants thereof. Further examples are described below.

Drugs

Non-limiting examples of drugs or payloads used in various embodiments of ADCs include DM1 (maytansine, N2′-deacetyl-N2′-(3-mercapto-1-oxopropyl)- or NT-deacetyl-N2′-(3-mercapto-1-oxopropyl)-maytansine), mc-MMAD (6-maleimidocaproyl-monomethylauristatin-D or N-methyl-L-valyl-N-[(1S,2R)-2-methoxy-4-[(2S)-2-[(1R,2R)-1-methoxy-2-methyl-3-oxo-3-[[(1S)-2-phenyl-1-(2-thiazolyl)ethyl]amino]propyl]-1-pyr rolidinyl]-1-[(1S)-1-methylpropyl]-4-oxobutyl]-N-methyl-(9C1)-L-valinamide), mc-MMAF (maleimidocaproyl-monomethylauristatin F or N-[6-(2,5-dihydro-2,5-dioxo-1H-pyrrol-1-yl)-1-oxohexyl]-N-methyl-L-valyl-L-valyl-(3R,4S,5S)-3-methoxy-5-methyl-4-(methylamino)heptanoyl-(αR,βR,2S)-β-methoxy-α-methyl-2-pyrrolidinepropanoyl-L-phenylalanine) and mc-Val-Cit-PABA-MMAE (6-maleimidocaproyl-ValcCit-(p-aminobenzyloxycarbonyl)-monomethylauristatin E or N-[[[4-[[N-[6-(2,5-dihydro-2,5-dioxo-1H-pyrrol-1-yl)-1-oxohexyl]-L-valyl-N5-(aminocarbonyl)-L-ornithyl]amino]phenyl]methoxy]carbonyl]-N-meth yl-L-valyl-N-[(1S,2R)-4-[(2S)-2-[(1R,2R)-3-[[(1R,2S)-2-hydroxy-1-methyl-2-phenylethyl]amino]-1-methoxy-2-methyl-3-oxopropyl]-1-pyrrolidinyl]-2-methoxy-1-[(1S)-1-methylpropyl]-4-oxobutyl]-N-methyl-L-valinamide). DM1 is a derivative of the tubulin inhibitor maytansine while MMAD, MMAE, and MMAF are auristatin derivatives.

Maytansinoid Drug Moieties

As indicated above, in some embodiments the drug is a maytansinoid. Exemplary maytansinoids include DM1, DM3 (N²′-deacetyl-N²′-(4-mercapto-1-oxopentyl) maytansine), and DM4 (N²′-deacetyl-N²′-(4-methyl-4-mercapto-1-oxopentyl)methylmaytansine) (see US20090202536).

Many positions on maytansine compounds are known to be useful as the linkage position, depending upon the type of link. For example, for forming an ester linkage, the C-3 position having a hydroxyl group, the C-14 position modified with hydroxymethyl, the C-15 position modified with a hydroxyl group and the C-20 position having a hydroxyl group are all suitable.

All stereoisomers of the maytansinoid drug moiety are contemplated for the ADCs described herein, i.e. any combination of R and S configurations at the chiral carbons of D.

Auristatins

In some embodiments, the drug is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Pat. Nos. 6,884,869, 7,098,308, 7,256,257, 7,423,116, 7,498,298 and 7,745,394, each of which is incorporated by reference herein in its entirety and for all purposes.

Chemotherapeutic Agents

In some embodiments the antigen binding construct is conjugated to a chemotherapeutic agent. Examples include but are not limited to Cisplantin and Lapatinib. A “chemotherapeutic agent” is a chemical compound useful in the treatment of cancer.

Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2, 2′,2′=-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

Conjugate Linkers

In some embodiments, the drug is linked to the antigen binding construct, e.g., antibody, by a linker. Attachment of a linker to an antibody can be accomplished in a variety of ways, such as through surface lysines, reductive-coupling to oxidized carbohydrates, and through cysteine residues liberated by reducing interchain disulfide linkages. A variety of ADC linkage systems are known in the art, including hydrazone-, disulfide- and peptide-based linkages.

Suitable linkers include, for example, cleavable and non-cleavable linkers. A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (mc-Val-Cit-PABA) linker. Another linker is Sulfosuccinimidyl-4-[N-maleimidomethyl]cyclohexane-1-carboxylate (SMCC). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Yet another linker is maleimidocaproyl (MC). Other suitable linkers include linkers hydrolyzable at a specific pH or a pH range, such as a hydrazone linker. Additional suitable cleavable linkers include disulfide linkers. The linker may be covalently bound to the antibody to such an extent that the antibody must be degraded intracellularly in order for the drug to be released e.g. the MC linker and the like.

Preparation of ADCs

The ADC may be prepared by several routes, employing organic chemistry reactions, conditions, and reagents known to those skilled in the art, including: (1) reaction of a nucleophilic group or an electrophilic group of an antibody with a bivalent linker reagent, to form antibody-linker intermediate Ab-L, via a covalent bond, followed by reaction with an activated drug moiety D; and (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) may be employed with a variety of antibodies, drug moieties, and linkers to prepare the antibody-drug conjugates described here.

Several specific examples of methods of preparing ADCs are known in the art and are described in U.S. Pat. No. 8,624,003 (pot method), U.S. Pat. No. 8,163,888 (one-step), and U.S. Pat. No. 5,208,020 (two-step method).

### Formulation of Antigen Binding Constructs and Administration Methods

The antigen binding constructs described herein can be formulated and administered by any method well known to one of skill in the art and depending on the application. In some embodiments the antigen-binding construct is formulated in a pharmaceutical composition of the antigen-binding construct and a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. In some aspects, the carrier is a man-made carrier not found in nature. Water can be used as a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In certain embodiments, the composition comprising the construct is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the antigen-binding constructs described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

Various delivery systems are known and can be used to administer an antigen-binding construct formulation described herein, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in certain embodiments, it is desirable to introduce the antigen-binding construct compositions described herein into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it is desirable to administer the antigen-binding constructs, or compositions described herein locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, described herein, care must be taken to use materials to which the protein does not absorb.

In another embodiment, the antigen-binding constructs or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the antigen-binding constructs or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, supra, vol. 2, pp. 115-138 (1984)).

In a specific embodiment comprising a nucleic acid encoding antigen-binding constructs described herein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

In certain embodiments a one arm monovalent antigen-binding construct described herein is administered as a combination with other one arm monovalent or multivalent antibodies with non-overlapping binding target epitopes.

Also provided herein are pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In certain embodiments, the composition comprising the antigen-binding constructs is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

The amount of the composition described herein which will be effective in the treatment, inhibition and prevention of a disease or disorder associated with aberrant expression and/or activity of a Therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

The antigen-binding constructs described herein may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in an embodiment, human antibodies, fragments derivatives, analogs, or nucleic acids, are administered to a human patient for therapy or prophylaxis.

### Polypeptides and Nucleic Acids

The methods described herein use isolated antigen binding constructs comprising polypeptides encoded by nucleic acids.

The term “isolated,” when applied to a nucleic acid or protein, denotes that the nucleic acid or protein is free of at least some of the cellular components with which it is associated in the natural state, or that the nucleic acid or protein has been concentrated to a level greater than the concentration of its in vivo or in vitro production. It can be in a homogeneous state. Isolated substances can be in either a dry or semi-dry state, or in solution, including but not limited to, an aqueous solution. It can be a component of a pharmaceutical composition that comprises additional pharmaceutically acceptable carriers and/or excipients. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to substantially one band in an electrophoretic gel. Particularly, it may mean that the nucleic acid or protein is at least 85% pure, at least 90% pure, at least 95% pure, at least 99% or greater pure.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins of the present invention may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles described herein.

Conservative substitution tables providing functionally similar amino acids are known to those of ordinary skill in the art. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and [0139] 8) Cysteine (C), Methionine (M) (see, e.g., Creighton, Proteins: Structures and Molecular Properties (W H Freeman & Co.; 2nd edition (December 1993)

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide of the present invention, including homologs from species other than human, may be obtained by a process comprising the steps of screening a library under stringent hybridization conditions with a labeled probe having a polynucleotide sequence described herein or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al. (1997) Nuc. Acids Res. 25:3389-3402, and Altschul et al. (1990) J. Mol. Biol. 215:403-410, respectively. Software for performing BLAST analyses is publicly available through the National Center for Biotechnology Information available at the World Wide Web at ncbi.nlm.nih.gov. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, or other nucleic acids, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993).

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides under discussion can be modified or unmodified.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

Provided are antigen-binding constructs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Additional post-translational modifications encompassed herein include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The antigen-binding constructs are modified with a detectable label, such as an enzymatic, fluorescent, isotopic, or affinity label to allow for detection and isolation of the protein. Examples of suitable enzymes include horseradish peroxidase, alkaline phosphatase, beta-galactosidase, or acetylcholinesterase; examples of suitable prosthetic group complexes include streptavidin biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine, carbon, sulfur, tritium, indium, technetium, thallium, gallium, palladium, molybdenum, xenon, fluorine.

Various publications are cited herein, the disclosures of which are incorporated by reference in their entireties.

It is to be understood that this invention is not limited to the particular protocols; cell lines, constructs, and reagents described herein and as such may vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing described herein, the preferred methods, devices and materials are now described.

All publications and patents mentioned herein are incorporated herein by reference for the purpose of describing and disclosing, for example, the constructs and methodologies that are described in the publications, which might be used in connection with the presently described invention. The publications discussed herein are provided solely for their disclosure prior to the filing date of the present application. Nothing herein is to be construed as an admission that the inventors are not entitled to antedate such disclosure by virtue of prior invention or for any other reason.

### REFERENCES

Bowles J A, Wang S Y, Link B K, Allan B, Beuerlein G, Campbell M A, Marquis D, Ondek B, Wooldridge J E, Smith B J, Breitmeyer J B, Weiner G J. Anti-CD20 monoclonal antibody with enhanced affinity for CD16 activates NK cells at lower concentrations and more effectively than rituximab. Blood. 2006 Oct. 15; 108(8):2648-54. Epub 2006 Jul. 6.
Desjarlais J R, Lazar G A. Modulation of antibody effector function. Exp Cell Res. 2011 May 15; 317(9):1278-85.
Ferrara C, Grau S, Jager C, Sondermann P, Brünker P, Waldhauer I, Hennig M, Ruf A, Rufer A C, Stihle M, Umaña P, Benz J. Unique carbohydrate-carbohydrate interactions are required for high affinity binding between FcgammaRIII and antibodies lacking core fucose. Proc Natl Acad Sci USA. 2011 Aug. 2; 108(31):12669-74.
Heider K H, Kiefer K, Zenz T, Volden M, Stilgenbauer S, Ostermann E, Baum A, Lamche H, Küpcū Z, Jacobi A, Müller S, Hirt U, Adolf G R, Borges E. A novel Fc-engineered monoclonal antibody to CD37 with enhanced ADCC and high proapoptotic activity for treatment of B-cell malignancies. Blood. 2011 Oct. 13; 118(15):4159-68. Epub 2011 Jul. 27. Blood. 2011 Oct. 13; 118(15):4159-68. Epub 2011 Jul. 27.
Lazar G A, Dang W, Karki S, Vafa O, Peng J S, Hyun L, Chan C, Chung H S, Eivazi A, Yoder S C, Vielmetter J, Carmichael D F, Hayes R J, Dahiyat B I. Engineered antibody Fc variants with enhanced effector function. Proc Natl Acad Sci USA. 2006 Mar. 14; 103(11):4005-10. Epub 2006 Mar. 6.
Lu Y, Verres J M, Chiang N, Ou Q, Ding J, Adams C, Hong K, Truong B T, Ng D, Shen A, Nakamura G, Gong Q, Presta L G, Beresini M, Kelley B, Lowman H, Wong W L, Meng Y G. Identification of IgG(1) variants with increased affinity to FcγRIIIa and unaltered affinity to FcγR I and FcRn: comparison of soluble receptor-based and cell-based binding assays. J Immunol Methods. 2011 Feb. 28; 365(1-2):132-41. Epub 2010 Dec. 23.
Mizushima T, Yagi H, Takemoto E, Shibata-Koyama M, Isoda Y, Iida S, Masuda K, Satoh M, Kato K. Structural basis for improved efficacy of therapeutic antibodies on defucosylation of their Fc glycans. Genes Cells. 2011 November; 16(11):1071-1080.
Moore G L, Chen H, Karki S, Lazar G A. Engineered Fc variant antibodies with enhanced ability to recruit complement and mediate effector functions. MAbs. 2010 March-April; 2(2):181-9.
Nordstrom J L, Gorlatov S, Zhang W, Yang Y, Huang L, Burke S, Li H, Ciccarone V, Zhang T, Stavenhagen J, Koenig S, Stewart S J, Moore P A, Johnson S, Bonvini E. Anti-tumor activity and toxicokinetics analysis of MGAH22, an anti-HER2 monoclonal antibody with enhanced Fc-gamma receptor binding properties. Breast Cancer Res. 2011 Nov. 30; 13(6):R123. [Epub ahead of print]
Richards J O, Karki S, Lazar G A, Chen H, Dang W, Desjarlais J R. Optimization of antibody binding to FcgammaRIIa enhances macrophage phagocytosis of tumor cells. Mol Cancer Ther. 2008 August; 7(8):2517-27.
Schneider S, Zacharias M. Atomic resolution model of the antibody Fc interaction with the complement Clq component. Mol Immunol. 2012 May; 51(1):66-72.
Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma RI, Fc gamma RII, Fc gamma RIII, and FcRn and design of IgG1 variants with improved binding to the Fc gamma R. J Biol Chem. 2001 Mar. 2; 276(9):6591-604.
Stavenhagen J B, Gorlatov S, Tuaillon N, Rankin C T, Li H, Burke S, Huang L, Vijh S, Johnson S, Bonvini E, Koenig S. Fc optimization of therapeutic antibodies enhances their ability to kill tumor cells in vitro and controls tumor expansion in vivo via low-affinity activating Fcgamma receptors. Cancer Res. 2007 Sep. 15; 67(18):8882-90.
Stewart R, Thom G, Levens M, Güler-Gane G, Holgate R, Rudd P M, Webster C, Jermutus L, Lund J. A variant human IgG1-Fc mediates improved ADCC. Protein Eng Des Sel. 2011 September; 24(9):671-8. Epub 2011 May 18.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

The reagents employed in the examples are generally commercially available or can be prepared using commercially available instrumentation, methods, or reagents known in the art. The foregoing examples illustrate various aspects described herein and practice of the methods described herein. The examples are not intended to provide an exhaustive description of the many different embodiments described herein. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

### Example 1

### Preparation of One-Armed (OA) Anti-HER2 Antibodies and Controls

A number of monovalent anti-HER2 antibodies and controls were prepared as described below. FIG. 1 depicts schematic representations of different OA antibody formats. FIG. 1A depicts the structure of a bivalent mono-specific, full-sized antibody, where the light chains are shown in white, the Fab portion of the heavy chain is shown in hatched fill, and the Fc portion of the heavy chains are grey. FIG. 1B depicts two versions of a monovalent, mono-specific OA where the antigen-binding domain is in the Fab format. In both of these versions, the light chain is shown in white, while the Fab portion of the heavy chain is shown in hatched fill. The Fc portion of Chain A is grey and the Fc portion of Chain B is black. In the version on the left, the Fab is fused to Chain A, while in the version on the right, the Fab is fused to Chain B. FIG. 1C depicts two versions of an OA where the antigen-binding domain is in the scFv format. In both of these versions, the variable domain of the light chain (VL) is shown in white, while the variable domain of the heavy chain (VH) is shown in hatched fill. The Fc portion of Chain A is grey and the Fc portion of Chain B is black. In the version on the left, the scFv is fused to Chain A, while in the version on the right, the scFv is fused to Chain B. A number of OA anti-HER2 antibodies in the formats described in FIG. 1B or FIG. 1C were prepared as described below and in Example 17.

Exemplary Anti-HER2 Monovalent Antibodies (One-Armed Antibodies, OAAs):

v1040: a monovalent anti-HER2 antibody, where the HER2 binding domain is a Fab derived from trastuzumab on chain A, and the Fc region is a heterodimer having the mutations T350V_L351Y_F405A_Y407V in Chain A, T350V_T366L_K392L_T394W in Chain B, and the hinge region of Chain B having the mutation C226S; the antigen binding domain binds to domain 4 of HER2. The DNA sequences of heavy chain A, light chain, and heavy chain B, respectively are provided as follows: SEQ ID NO: 11, SEQ ID NO: 13 and SEQ ID NO: 15; The amino acid sequences of heavy chain A, light chain, and heavy chain B, respectively are provided as follows: SEQ ID NO: 12, SEQ ID NO: 14, and SEQ ID NO: 16.

v4182: a monovalent anti-HER2 antibody, where the HER2 binding domain is a Fab derived from pertuzumab on chain A, and the Fc region is a heterodimer having the mutations T350V_L351Y_F405A_Y407V in Chain A, T350V_T366L_K392L_T394W in Chain B, and the hinge region of Chain B having the mutation C226S; the antigen binding domain binds to domain 2 of HER2.

Control Anti-HER2 Bivalent Antibodies (Full-Sized Antibodies, FSAs)

v506 is a wild-type anti HER2 produced in-house in Chinese Hamster Ovary (CHO) cells, as a control. Both HER2 binding domains are derived from trastuzumab in the Fab format and the Fc is a wild type homodimer; the antigen binding domain binds to domain 4 of HER2. This antibody is also referred to as a trastuzumab analog.

v792, is wild-type trastuzumab with a IgG1 hinge, where both HER2 binding domains are derived from trastuzumab in the Fab format, and the and the Fc region is a heterodimer having the mutations T350V_L351Y_F405A_Y407V in Chain A, and T350V_T366L_K392L_T394W Chain B; the antigen binding domain binds to domain 4 of HER2. This antibody is also referred to as a trastuzumab analog.

v4184, a bivalent anti-HER2 antibody, where both HER2 binding domains are derived from pertuzumab in the Fab format, and the Fc region is a heterodimer having the mutations L351Y_S400E_F405A_Y407V in Chain A, and T366I_N390R_K392M_T394W in Chain B. The antigen binding domain binds to domain 2 of HER2. This antibody is also referred to as a pertuzumab analog.

hIgG, is a commercial non-specific polyclonal antibody control (Jackson ImmunoResearch, #009-000-003).

The relevant amino acid and DNA sequences corresponding to each variant are shown in Table 1.

TABLE 1

| Variant | Type | Heavy Chain A | Heavy Chain B | Light chain |
|---|---|---|---|---|
| v1040 | Amino acid | SEQ ID NO: 12 | SEQ ID NO: 16 | SEQ ID NO: 14 |
| | DNA | SEQ ID NO: 11 | SEQ ID NO: 15 | SEQ ID NO: 13 |
| v4182 | Amino acid | SEQ ID NO: 40 | SEQ ID NO: 42 | SEQ ID NO: 44 |
| | DNA | SEQ ID NO: 39 | SEQ ID NO: 41 | SEQ ID NO: 43 |
| v506 | Amino acid | SEQ ID NO: 23 | — | SEQ ID NO: 25 |
| | DNA | SEQ ID NO: 24 | — | SEQ ID NO: 26 |
| v792 | Amino acid | SEQ ID NO: 28 | SEQ ID NO: 32 | SEQ ID NO: 30 |
| | DNA | SEQ ID NO: 27 | SEQ ID NO: 31 | SEQ ID NO: 29 |
| v4184 | Amino acid | SEQ ID NO: 52 | SEQ ID NO: 54 | SEQ ID NO: 56 |
| | DNA | SEQ ID NO: 51 | SEQ ID NO: 53 | SEQ ID NO: 55 |

These antibodies and controls were cloned and expressed as follows. The genes encoding the antibody heavy and light chains were constructed via gene synthesis using codons optimized for human/mammalian expression. The Trastuzumab Fab sequence was generated from a known HER2/neu domain 4 binding antibody (Carter P. et al. (1992) Humanization of an anti p185 her2 antibody for human cancer therapy. Proc Natl Acad Sci 89, 4285.) nd the Fc was an IgG1 isotype. The Pertuzumab Fab sequence was generated from a known HER2/neu domain 2 binding antibody (Adams C W et al. (2006) Humanization of a recombinant monoclonal antibody to produce a therapeutic HER2 dimerization inhibitor, Pertuzumab. Cancer Immunol Immunother. 2006; 55(6):717-27).

The final gene products were sub-cloned into the mammalian expression vector PTT5 (NRC-BRI, Canada) and expressed in CHO cells (Durocher, Y., Perret, S. & Kamen, A. High-level and high-throughput recombinant protein production by transient transfection of suspension-growing CHO cells. Nucleic acids research 30, e9 (2002)).

The CHO cells were transfected in exponential growth phase (1.5 to 2 million cells/ml) with aqueous 1 mg/ml 25 kDa polyethylenimine (PEI, polysciences) at a PEI:DNA ratio of 2.5:1. (Raymond C. et al. A simplified polyethylenimine-mediated transfection process for large-scale and high-throughput applications. Methods. 55(1):44-51 (2011)). To determine the optimal concentration range for forming heterodimers, the DNA was transfected in optimal DNA ratios of the heavy chain a (HC-A), light chain (LC), and heavy chain B (HC-B) that allow for heterodimer formation (e.g. HC-A/HC-B/LC ratios=30:30:40 (v1040 or v4182). Transfected cells were harvested after 5-6 days with the culture medium collected after centrifugation at 4000 rpm and clarified using a 0.45 μm filter.

The clarified culture medium was loaded onto a MabSelect™ SuRe (GE Healthcare) protein-A column and washed with 10 column volumes of PBS buffer at pH 7.2. The antibody was eluted with 10 column volumes of citrate buffer at pH 3.6 with the pooled fractions containing the antibody neutralized with TRIS at pH 11.

The protein-A antibody eluate was further purified by gel filtration (SEC). For gel filtration, 3.5 mg of the antibody mixture was concentrated to 1.5 mL and loaded onto a Sephadex 200 HiLoad 16/600 200 pg column (GE Healthcare) via an AKTA Express FPLC at a flow-rate of 1 mL/min. PBS buffer at pH 7.4 was used at a flow-rate of 1 mL/min. Fractions corresponding to the purified antibody were collected, concentrated to ˜1 mg/mL.

### Example 2

### Preparation of Exemplary Anti-HER2 Antibody Drug Conjugates (ADCs)

The following anti-HER2 antibody drug conjugates were prepared: v6246: v506 conjugated to DM1 (T-DM1 analog); v6247: v1040 (OA-tras) conjugated to DM1; v6248: v4182 (OA-pert) conjugated to DM1.

These ADCs were prepared via direct coupling to the maytansine. Antibodies purified by Protein A and SEC as described in Example 1 (>95% purity) were used in the preparation of the ADC molecules. ADCs were conjugated following the method described in Kovtun Y V, Audette C A, Ye Y, et al. Antibody-drug conjugates designed to eradicate tumors with homogeneous and heterogeneous expression of the target antigen. Cancer Res 2006; 66:3214-21. The ADCs had an average molar ratio of 2.8 to 3.5 maytansinoid molecules per antibody as determined by LC/MS as described below.

Details of the reagents used in the ADC conjugation reaction are as follows: Conjugation Buffer 1: 50 mM Potassium Phosphate/50 mM Sodium Chloride, pH 6.5, 2 mM EDTA. Conjugation Buffer 2: 50 mM Sodium Succinate, pH 5.0. ADC formulation buffer: 20 mM Sodium Succinate, 6% (w/v) Trehalose, 0.02% polysorbate 20, pH 5.0. Dimethylacetamide (DMA); 10 mM SMCC in DMA (prepared before conjugation), 10 mM DM1-SH in DMA (prepared before conjugation),) mM DTNB in PBS, 1 mM Cysteine in buffer, 20 mM Sodium Succinate, pH 5.0. UV-VIS spectrophotometer (Nano drop 100 from Fisher Scientific), PD-10 columns (GE Healthcare).

The ADCs were prepared as follows. The starting antibody solution was loaded onto the PD-10 column, previously equilibrated with 25 mL of Conjugation Buffer 1, followed by 0.5 ml Conjugation Buffer 1. The antibody eluate was collect and the concentration measured at A₂₈₀ and the concentration was adjusted to 20 mg/mL. The 10 mM SMCC-DM1 solution in DMA was prepared. A 7.5 molar equivalent of SMCC-DM1 to antibody was added to the antibody solution and DMA was added to a final DMA volume of 10% v/v. The reaction was briefly mixed and incubated at RT for 2 h. A second PD-10 column was equilibrated with 25 ml of Conjugation Buffer 1 and the antibody-SMCC-DM1 solution was added to the column follow by 0.5 ml of Buffer 1. The antibody-SMCC-DM1 eluate was collected and the A₂₅₂ and A₂₈₀ of antibody solution was measured. The Antibody-SMCC-DM1 concentration was calculated (=1.45 mg⁻¹ cm⁻¹, or 217500 M⁻¹ cm⁻¹). The ADCs were analyzed on a SEC-HPLC column for high MW analysis (SEC-HPLC column TOSOH, G3000-SWXL, 7.8 mm×30 cm, Buffer, 100 mM Sodium phosphate, 300 mM Sodium Chloride, pH 7.0, flow rate: 1 ml/min).

ADC drug to antibody ratio (DAR) was determined by LC/MS by the following method. The antibodies were deglycosylated with PNGasF prior to loading on the LC-MS. Liquid chromatography was carried out on an Agilent 1100 Series HPLC under the following conditions:

Flow rate: 1 mL/min split post column to 100 uL/min to MS. Solvents: A=0.1% formic acid in ddH2O, B=65% acetonitrile, 25% THF, 9.9% ddH2O, 0.1% formic acid. Column: 2.1×30 mm PorosR2. Column Temperature: 80° C.; solvent also pre-heated. Gradient: 20% B (0-3 min), 20-90% B (3-6 min), 90-20% B (6-7 min), 20% B (7-9 min)

Mass Spectrometry (MS) was subsequently carried out on an LTQ-Orbitrap XL mass spectrometer under the following conditions: Ionization method using Ion Max Electrospray. Calibration and Tuning Method: 2 mg/mL solution of CsI is infused at a flowrate of 10 μL/min. The Orbitrap is then tuned on m/z 2211 using the Automatic Tune feature (overall CsI ion range observed: 1690 to 2800). Cone Voltage: 40V; Tube Lens: 115V; FT Resolution: 7,500; Scan range m/z 400-4000; Scan Delay: 1.5 min. A molecular weight profile of the data was generated using Thermo's Promass deconvolution software. DAR was determined using the calculation: Σ(DAR× fractional peak intensity).

Table 2 summarizes the average DAR for the ADC molecules. The average DAR for the OA anti-HER2 ADCs was approximately 2, and the average DAR for the anti-HER2 FSA was 3.4.

TABLE 2

| ADC variant | DAR determined by LC/MS |
|---|---|
| v6247 | 1.9 |
| v6248 | 2.1 |
| v6246 | 3.4 |

### Example 3

### Measurement of Cell Surface Binding by Monovalent Anti-HER2 Antibodies and Combinations Thereof Using FACS

The following experiment was performed in order to measure the amount of monovalent anti-HER2 antibodies bound to the surface of SKOV3 cells, an ovarian HER2 2-3+(gene amplified) cell line expressing high levels of HER2. The experiment was carried out as follows.

Binding of the test antibodies to the surface of SKOV3 cells was determined by flow cytometry. Cells were grown to subconfluency and washed with PBS and resuspended in DMEM at 1×10⁵ cells/100 μl. 100 μl cell suspension was added into each microcentrifuge tube, followed by 10 μl/tube of the antibody variants. The tubes were incubated for 2 hr 4° C. on a rotator. The microcentrifuge tubes were centrifuged for two minutes at 2000 RPM at room temperature and the cell pellets washed with 500 μl media. Each cell pellet was resuspended 100 μl of fluorochrome-labelled secondary antibody diluted in media to 2 μg/sample. The samples were then incubated for 1 hr at 4° C. on a rotator. After incubation, the cells were centrifuged for 2 min at 2000 rpm and washed in media. The cells were resuspended in 500 μl media, filtered, and transferred to tube containing 5 μl propidium iodide (PI) and analyzed on a BD lsrii flow cytometer according to the manufacturer's instructions. The K_{D} of exemplary biparatopic anti-HER2 heterodimer antibody and control antibodies were assessed by FACS with data analysis and curve fitting performed in GraphPad Prism.

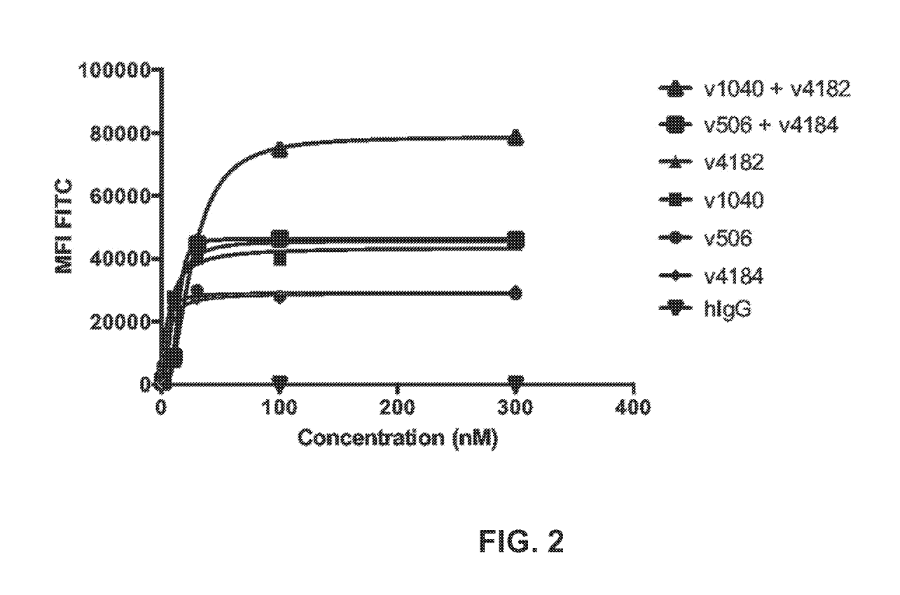

The results are shown in FIG. 2 and summarized in Table 3 below.

TABLE 3

| Antibody variant | KD (nM) | Bmax (MFI) |
|---|---|---|
| v506 | 2.713 | 29190 |
| v4184 | 4.108 | 29188 |
| v506 + v4184 | 13.85 | 46279 |
| v1040 | 6.058 | 43668 |
| v4182 | 10.00 | 45790 |
| v1040 + v4182 | 26.04 | 78874 |

The FACS binding results in FIG. 2 and the summarized data in Table 2 show that the combination of two anti-HER2 OAAs (v1040+v4182) have increased whole cell binding (Bmax) that is approximately 1.7-fold greater than the Bmax of individual OAAs (v1040, v4182) and the FSA combination (v506+v4184), and approximately 2.7-fold greater than the Bmax of the FSA antibody (v506). Apparent K_{D} values show that the combination of two anti-HER2 OAA, have an approximate 2-fold higher K_{D} compared to the FSA combination and approximate 10-fold higher K_{D} compare to the FSA v506.

### Example 4

### Measurement of Cell Surface Binding by Monovalent Anti-HER2 Antibodies and Combinations Thereof by Confocal Microscopy

The ability of monovalent anti-HER2 antibodies and combinations thereof to bind to the surface of JIMT-1 cells (a trastuzumab-resistant breast cancer cell line) was measured using confocal microscopy. Confocal microscopy was used in order to visualize whole cell binding over different time points.

JIMT-1 cells were incubated with the antibody variants (200 nM) in serum-free DMEM, 37° C. for 1 h, 3 h and 16 h. Cells were gently washed two times with warmed sterile PBS (500 ml/well). Cells were fixed with 250 ml of 10% formalin/PBS solution for 10 mins at room temperature. The formalin solution was removed and fixed cells washed three times with PBS (500 ul/well). Cells were permeabilized with 250 μl/well of PBS containing 0.2% Triton X-100 for 5 min, then washed three times with 500 μl/well PBS. Blocking was carried out with 500 μl/well of PBS+5% goat serum for 1 h at room temperature. The blocking buffer was removed, and 300 μl/well secondary antibodies was added and the cells incubated at room temperature for 1 h. The secondary antibodies were removed by washing three times with 500 μl/well of PBS. The coverslips containing fixed cells were then mounted on a slide using Prolong gold anti-fade with DAPI/Life technologies/#P36931/lot #1319493). 60× single images were acquired using Olympus FV1000 Confocal microscope.

The results of this experiment show that combination of two anti-HER2 one-armed antibodies (v1040+v4182) resulted in intense surface staining of the JIMT-1 cells at 1, 3 and 16 hours (overnight), compared to the FSA antibody (v506) and FSA combination (v506+v4184) which appeared to internalize at 1 and 3 h as denoted by punctate intracellular staining (data not shown). The surface staining of the OAA combinations (v1040+v4182) was greater than the surface staining of the individual OAA alone (v1040 or v4182) at all timepoints. The confocal cell staining images in JIMT-1 cells are consistent with the increased cell surface decoration (Bmax) data of the two anti-HER2 OAA combinations in SKOV3 cells, described in Example 3.

### Example 5

### Ability of Monovalent Anti-HER2 Antibodies and Combinations Thereof to Inhibit Cell Growth

This experiment was performed to measure the ability of monovalent anti-HER2 antibodies and combinations thereof to inhibit the growth of SKOV3 cells and BT-474 cells. As indicated previously, SKOV3 cells are an ovarian HER2 2-3+(gene amplified) cell line. BT-474 cells are a HER2 3+ breast cancer cell line. The experiments were carried out as described below.

Test antibodies were diluted in media and added to the SKOV3 or BT-474 cells at 10 μl/well (300 nM) in triplicate. The plates were incubated for 5 days 37° C. Cell viability was measured using AlamarBlue™ (BIOSOURCE # DAL1100). 10 μl/of AlamarBlue™ was added per well and the plates incubate at 37° C. for 2 hr. Absorbance was read at 530/580 nm. [controls?]

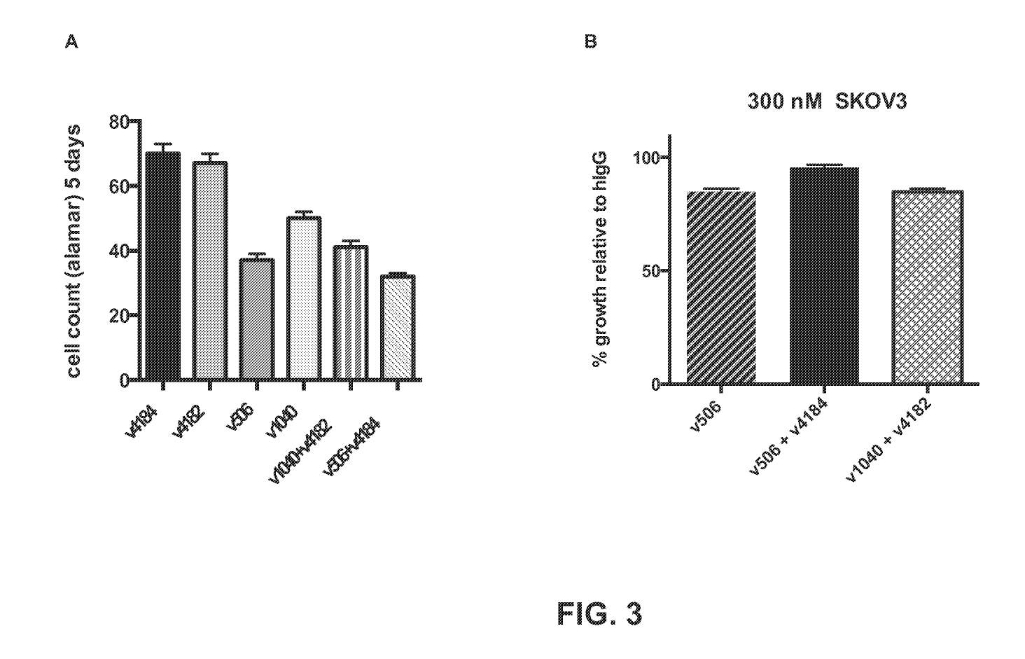

The results of the growth inhibition assay in BT-474 cells are found in FIG. 3A. These results show that combination of v1040+v4182 mediates greater growth inhibition compared to the individual OAA alone (v1040 or v4182), similar growth inhibition compared to the FSA v506, and less growth inhibition compared to the FSA combination (v506+v4184).

The results of the growth inhibition assay in SKOV3 cells are found in FIG. 3B. The data is reported as % growth relative to IgG control. Combination of v1040 and v4182 has equivalent growth inhibition compared to FSA v506 and superior growth inhibition compared to the FSA combination v506+v4184 in SKOV3.

The preceding data shows that the combination of monovalent anti-HER2 antibodies is capable of inhibiting the growth of HER2 3+ breast cancer cells and HER2 2-3+ ovarian cancer cells. However, there are differences in the level of growth inhibition observed between the HER2 3+ breast cancer cells and HER2 2-3+ ovarian cancer cells tested.

### Example 6

### Ability of Monovalent Anti-HER2 Antibodies and Combinations Thereof to Internalize in HER2+ Cells

This experiment was performed in order to determine the ability of monovalent anti-HER2 antibody antibodies and combinations to internalize compared to FSA and combinations. The assay was carried out in a HER2 3+ ovarian tumor cell line, SKOV3. The assay was carried out as follows.

The direct internalization method was followed according to the protocol detailed in Schmidt, M. et al., Kinetics of anti-carcinoembryonic antigen antibody internalization: effects of affinity, bivalency, and stability. Cancer Immunol Immunother (2008) 57:1879-1890. Specifically, the antibodies were directly labeled using the AlexaFluor® 488 Protein Labeling Kit (Invitrogen, cat. no. A10235), according to the manufacturer's instructions.

For the internalization assay, 12 well plates were seeded with 1×10⁵ cells/well and incubated overnight at 37° C.+5% CO2. The following day, the labeled antibodies were added at 200 nM in DMEM+10% FBS and incubated 24 hours at 37° C.+5% CO2. Under dark conditions, media was aspirated and wells were washed 2×500 μL PBS. To harvest cells, cell dissociation buffer was added (250 μL) at 37° C. Cells were pelleted and resuspended in 100 μL DMEM+10% FBS without or with anti-Alexa Fluor 488, rabbit IgG fraction (Molecular Probes, Al1094, lot 1214711) at 50 μg/mL, and incubated on ice for 30 min. Prior to analysis 300 μL DMEM+10% FBS the samples filtered 4 μL propidium iodide was added. Samples were analyzed using the LSRII flow cytometer.

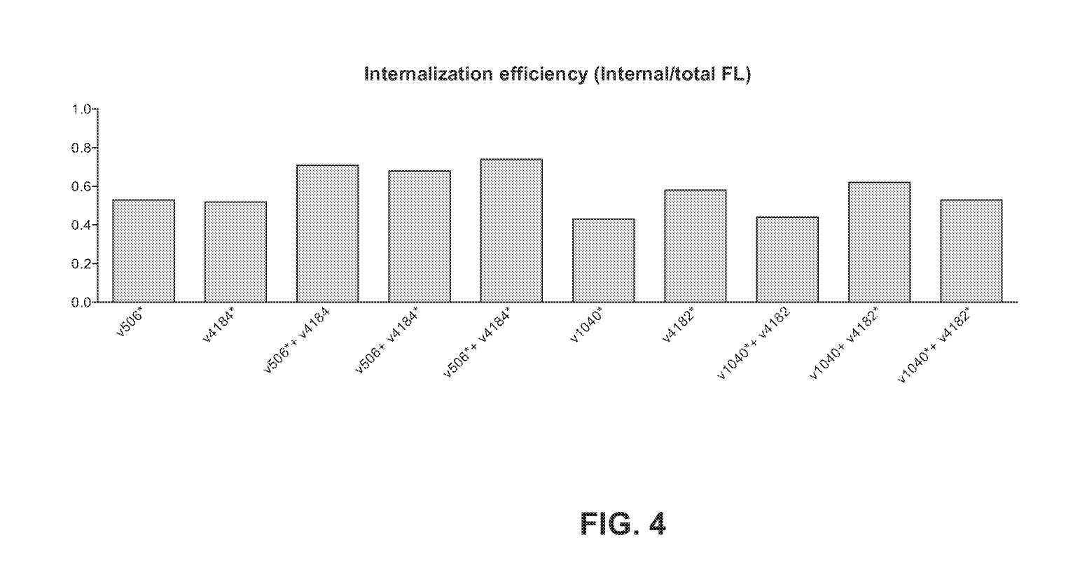

The results are shown in FIG. 4, where an asterisk * indicates an antibody that is fluorescently labeled and where the reported internalization efficacy is a measure of the amount of the labeled antibody that is internalized (e.g. ‘v1040*+v4182’ measures the amount of labeled v1040 that is internalized in the presence of v4182). All single antibody treatments were measured at 200 nM and the combination treatments were measured at 200 nM+200 nM. The results show that both anti-HER2 OAAs can internalize in HER2 3+SKOV3 cells.

### Example 7

### Ability of Monovalent Anti-HER2 Antibodies and Combinations to Mediate ADCC in HER2+ Cells

The following experiment was performed in order to assess the ability of monovalent anti-HER2 antibodies and combinations to mediate concentration-dependent ADCC in the SKOV3 cell line. The monovalent antibodies tested were 1040, the combination of 1040 and 4182, with 792 and 4184 as the FSA controls. All antibodies tested had comparable levels of fucosylation (approximately 88%) of the Fc N-linked glycan, as measured by glycopeptide analysis and detection by nanoLC-MS (data not shown). The assay was carried out as follows.

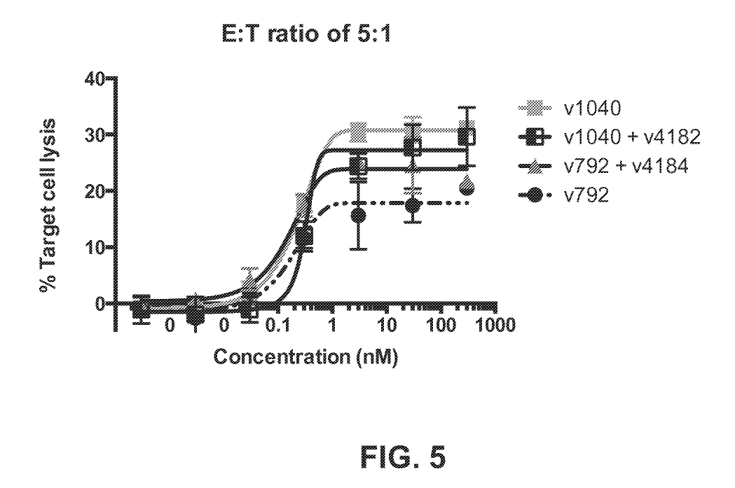

SKOV3 target cells (ATCC, Cat #HTB-30) were harvested by centrifugation at 800 rpm for 3 minutes. The cells were washed once with assay medium and centrifuged; the medium above the pellet was completely removed. The cells were gently suspended with assay medium to make single cell solution. The number of SKOV3 cells was adjusted to 4× cell stock (10,000 cells in 50 μl assay medium). The test antibodies were then diluted to the desired concentrations as noted in FIG. 5.

The SKOV3 target cells were seeded in the assay plates as follows. 50 μl of 4× target cell stock and 50 μl of 4× sample diluents was added to wells of a 96-well assay plate and the plate was incubated at room temperature for 30 min in cell culture incubator. Effector cells (NK92/FcRγ3a(158V/V), 100 μl, E/T=5:1, i.e, 50,000 effector cells per well) were added to initiate the reaction and mixed gently by cross shaking. The plate was incubated at 37° C./5% CO2 incubator for 6 hours.

Triton X-100 was added to cell controls without effector cells and antibody in a final concentration of 1% to lyse the target cells and these controls served as the maximum lysis controls. ADCC assay buffer (98% Phenol red free MEM medium, 1% Pen/Strep and 1% FBS) was added in to cell controls without effector cells and antibody and it served as the minimum LDH release control. Target cells incubated with effector cells without the presence of antibodies were set as background control of non-specific LDH release when both cells were incubated together. Cell viability was assayed with an LDH kit (Roche, cat#11644793001). The absorbance data was read at OD492 nm and OD650 nm on Flexstation 3. Data analysis and curve fitting (sigmoidal dose-response, variable slope) was performed in GraphPad Prism.

The results are shown in FIG. 5 and a summary of the results is provided in Table 4 below. The data in FIG. 5 and Table 4 show that the combination of two anti-HER2 OAAs can mediate approximately 1.5-fold greater percentage of maximum cell lysis by ADCC compared to an anti-HER2 FSA (v792, which differs from v506 in that it includes amino acid modifications to the Fc region, see description in Example 1) and approximately 1.1-fold greater ADCC compared to an anti-HER2 FSA combination (v792+v4184). The percent maximum cell lysis was approximately equivalent between the OA alone (v1040) and the OAA combination (v1040+v4182).

TABLE 4

| Antibody variant | IC50 (nM) SKOV3 | % Max Cell Lysis |
|---|---|---|
| v792 | ~0.032 | 18 |
| v792 + v4184 | ~0.04 | 24 |
| v1040 | ~0.039 | 31 |
| v1040 + v4182 | 2.01 | 27 |

### Example 8

### Monovalent Anti-HER2 Antibody Drug Conjugates (ADC) in Combination Increase the Potency in HER2 2+ Cellular Cytotoxicity Over the Monovalent Anti-HER2 ADCs Alone

The ability of a combination of monovalent anti-HER2 antibodies conjugated to DM1 to mediate cellular cytotoxicity in a concentration-dependent manner was measured in HER2 2-3+ ovarian tumor (SKOV3), and HER2 2+ breast tumor (JIMT-1) cells. The assay was carried out as follows.

Test antibodies were diluted in media and added to the cells at 10 μl/well in triplicate. The plates were incubated for 4 days 37° C. Cell viability was measured using AlamarBlue™ (BIOSOURCE # DAL1100). 10 μl/of AlamarBlue™ was added per well and the plates incubate at 37° C. for 2 hr. Absorbance was read at 530/580 nm.

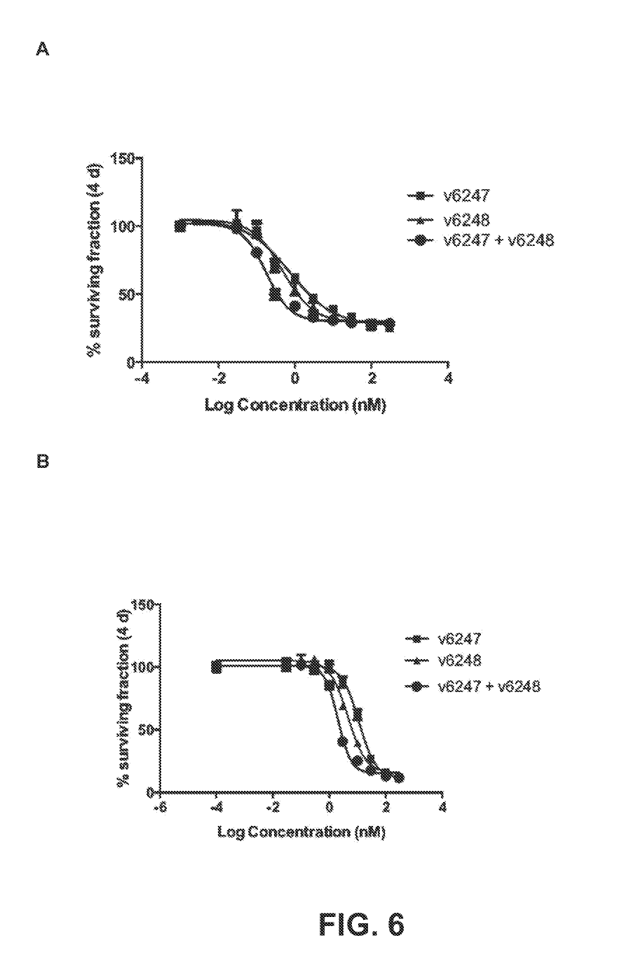

The results are shown in FIG. 6A (SKOV3 cells) and FIG. 6B (JIMT-1 cells) and a summary of the results is provided in Table 5. The results in FIG. 6A, FIG. 6B and Table 5 show that the combination of two anti-HER2 OAA (v6247+v6248) is approximately 2-3 to 4-fold more cytotoxic compared to the single OAAs alone (v6247 or v6248) at equimolar concentrations in SKOV3 as indicated by the IC₅₀. In JIMT-1 cells, the combination of two anti-HER2 OAAs (v6247+v6248) is approximately 2 to 6-fold more cytotoxic compared to the single OAAs alone (v6247 or v6248) at equimolar concentrations as indicated by the Log EC₅₀.

TABLE 5

| ADC variant | IC50 (nM) JIMT-1 | IC50 (nM) SKOV3 |
|---|---|---|
| v6247 | 11.3 | 0.751 |
| v6248 | 4.62 | 0.483 |
| v6247 + v6248 | 2.05 | 0.177 |

### Example 9

### Monovalent Anti-HER2 Antibody Drug Conjugates (ADC) in Combination Increase the Potency in HER2+ Cellular Cytotoxicity Over FSA-Tras-DM1

The effect of a combination of monovalent anti-HER2 ADCs on cellular cytotoxicity was measured in the Herceptin™ resistant HER2 2+ breast tumor cell line, JIMT-1 and compared to the FSA-Tras-DM1 (v6246, T-DM1 analog), and the FSA combination of FSA-Tras-DM1 and v4184 (Pertuzumab analog). The assay was performed as described for JIMT-1 cells in Example 8, except the cells were incubated with the OA ADCs for 5 days.

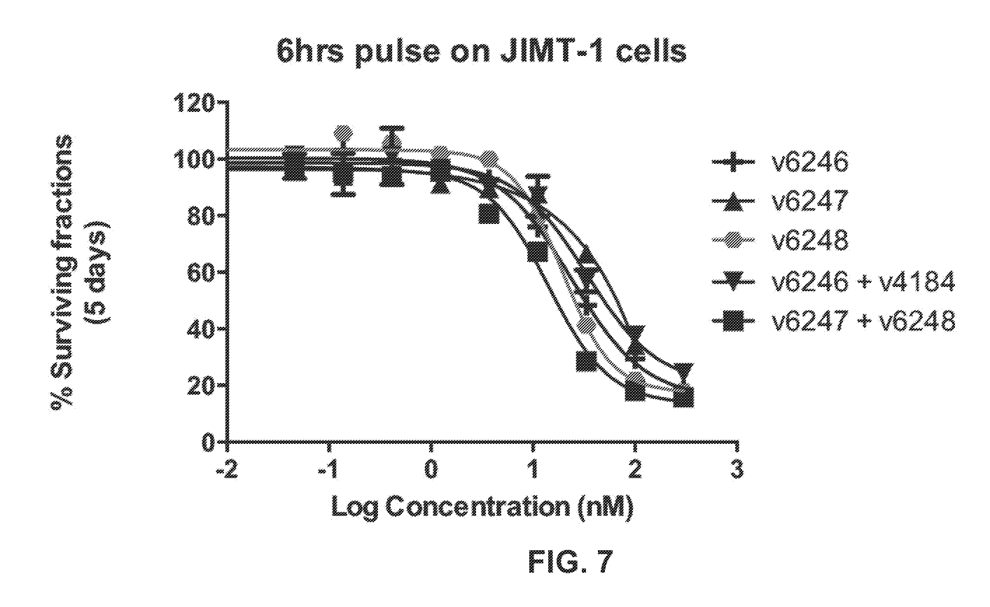

The results are shown in FIG. 7 and a summary of the results is provided in Table 6 below. Cytotoxicity of the individual OA ADCs (v6247 or v6248) was comparable to T-DM1 analog (v6246) and the T-DM1+Pertuzumab analog (v6246+v4184). The anti-HER2-ADC OAA combination (v6247+v6248) had the lowest IC50 value among the 5 ADC treatments (Table 6) and was approximately 2-fold more cytotoxic compared to the T-DM1+Pertuzumab analog (v6246+v4184) treatment.

TABLE 6

| ADC variant | IC50 (nM) JIMT-1 |
|---|---|
| v6246 | 23.8 |
| v6247 | Not determined |
| v6248 | 19.3 |
| v6247 + v6248 | 14.3 |
| v6246 + v4184 | 33.5 |

### Example 10

### Monovalent Anti-HER2 Antibody Combinations are More Effective in Inhibiting Established Tumor Growth in an SKOV3 Mouse Model Relative to IgG Control

This experiment was performed in order to determine the efficacy of monovalent anti-HER2 antibody as single agents and as follow-on combinations on tumor growth inhibition in an ovarian cancer cell derived xenograft model, SKOV3 (HER2 3+), that is moderately sensitive to Trastuzumab in nude mice. The effect of OA-Trastuzumab (v1040) was compared to Trastuzumab analog (v506) alone and in follow-on combination with either a OA-Pertuzumab (4182) or Pertuzumab analog (4184), respectively.

Female athymic nude mice were inoculated with the tumor via the insertion of a 1 mm³ tumor fragment subcutaneously. Tumors were monitored until they reached an average volume of 220 mm³; animals were then randomized into 3 treatment groups: IgG control, Trastuzumab analog (v506), and OA-Tras (v1040). Fifteen animals were included in each group. Dosing for each group was as indicated below or until tumour volumes reached 2000 mm³ (termination endpoint), which ever occurred first:

A) IgG control was dosed intravenously with a loading dose of 30 mg/kg on study day 1 then with maintenance doses of 20 mg/kg twice per week to study day 39.

B) Trastuzumab analog (v506) was dosed intravenously with loading doses of 15 mg/kg on study day 1 then with maintenance doses of 10 mg/kg twice per week to study day 18. On days 22 through 39, 5 mg/kg trastuzumab analog was dosed intravenously twice per week in combination with Pertuzumab analog (v4184) at 5 mg/kg intraperitoneally twice per week.

C) OA-Tras (v1040) was dosed intravenously with loading doses of 15 mg/kg on study day 1 then with maintenance doses of 10 mg/kg twice per week to study day 18. On study days 22 through 39, 10 mg/kg One-Armed trastuzumab was dosed intravenously twice per week in combination with OA-Pert (v4182) at 10 mg/kg intraperitoneally twice per week.

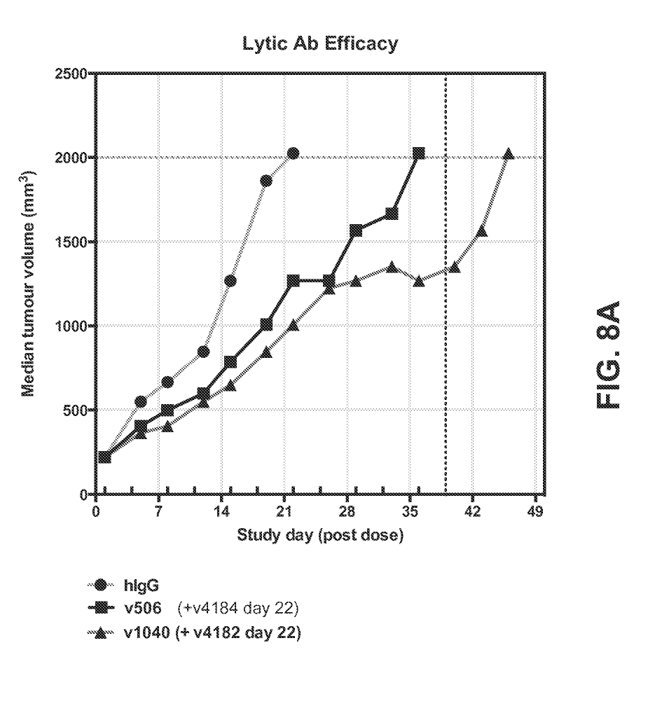

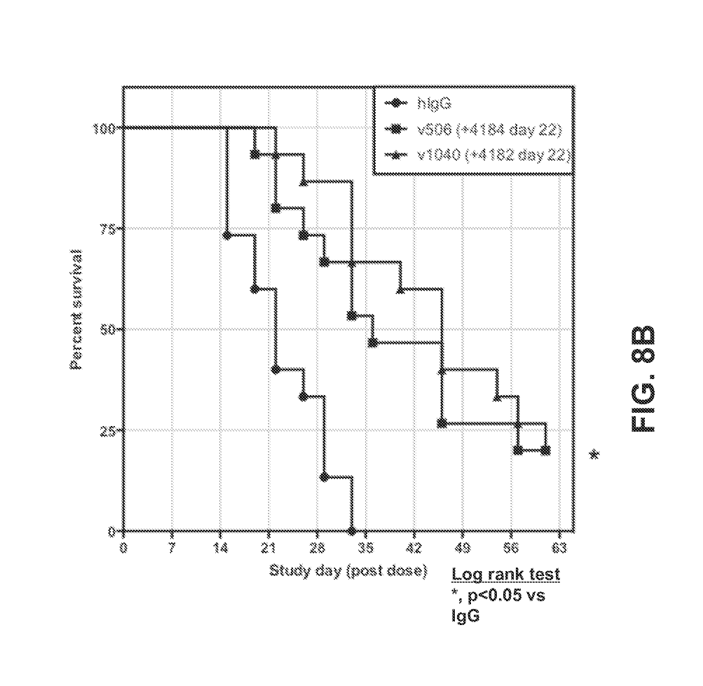

The results are shown in FIG. 8A, FIG. 8B and Tables 7 and 8. The OA-Trastuzumab monovalent antibody and the trastuzumab analog induced significant and similar tumor growth inhibition compared to IgG control. In addition, treatment with OA-Trastuzumab was associated with an increase in the number of tumors responding to therapy compared to Trastuzumab (7/15 vs 5/15, respectively) and a single animal that had zero residual disease (Table 7). Consistent with the PK results the serum exposure of the OA-Trastuzumab antibody on study day 11 was lower than the Trastuzumab analogue with values of 70.9 and 146.7 μg/ml respectively (Table 7).

As indicated above, on study day 22 either a pertuzumab analog was added in combination to the trastuzumab analog or a OA-Pertuzumab monovalent antibody was added in combination to the OA-Trastuzumab monovalent antibody. The combination of two anti-HER2 OAAs (v1040+v4182) showed an improved tumor growth inhibition as seen by a slower rate of tumor growth post combination dosing. Significant differences in tumor growth inhibition were not detected between v506 and v1040 treatment groups, nor between the combination groups (i.e. v1040+v4182, and v506+v4184) post day 22. The combination of two anti-HER2 OAA (v1040+v4182) showed a significant improvement in median survival vs control IgG (46 vs 22 days, respectively) and improved median survival compared to the combination of full sized antibodies (v506+v4184) with values of 46 vs 36 days, respectively (Table 8). Both therapeutic combinations significantly improved survival vs control and a trend towards superior survival was observed for the combination of two anti-HER2 OAA (v1040+v4182) compared to the combination of full sized antibodies (v506+v4184) (FIG. 8B). This result indicates that OA anti-HER2 antibodies may have therapeutic utility in HER2 positive ovarian cancers as a single agent and as OA anti-HER2 antibody combinations.

TABLE 7

| Day 22, n = 15 | IgG | v506 | v1040 |
|---|---|---|---|
| Mean TV (mm3) | 1908 | 1291 | 1194 |
| (% change from Baseline) | (+766%) | (+486%) | (+446%) |
| T/C (IgG) ratio | 1 | 0.68 | 0.62 |
| Responders | 0/15 | 5/15 | 7/15 |
| (TV <50% of control) | | | |
| Complete response | 0/15 | 0/15 | 0/15 |
| (>10% baseline regression) | | | |
| Zero residual disease | 0/15 | 0/15 | 1/15 |
| (TV <20 mm3) | | | |

TABLE 8

| Day 61, n = 15 | IgG | v506 + v4184 | v1040 + v4182 |
|---|---|---|---|
| Median Survival | 22 | 36 | 46 |
| (days) | | | |
| Day 11 Serum exposure | na | 146.7 | 70.9 |
| (microg/ml | | | |

### Example 11

### Efficacy of a Monovalent Anti-HER2 Antibody in Inhibiting Tumor Growth in a Primary Breast Cancer Xenograft Model HBCx-13b

This experiment was performed to compare the efficacy of a monovalent anti-HER2 antibody, to a full-sized Trastuzumab analog, in the trastuzumab resistant primary breast cancer xenograft model HBCx-13b. HBCx-13b is a HER2 3+, estrogen receptor negative, metastatic breast cancer that is innately resistant to Trastuzumab in nude mice. HBCx-13b is also resistant to docetaxel, capecitabine, and the combination of Adriamycin/Cyclophosphamide.

Female athymic nude mice were inoculated with the tumor via the insertion of a 20 mm³ tumor fragment subcutaneously. Tumors were monitored until they reached an average volume of 100 mm³; animals were then randomized into 2 treatment groups: trastuzumab analog (v506) and OA-tras (v1040). Seven animals were included in each group. Both groups were dosed intravenously with a loading dose of 15 mg/kg on study day 1 and maintenance doses of 10 mg/kg administered on study days 3, 7, 10, 14, 17, 21, and 24. Total study duration was 64 days.

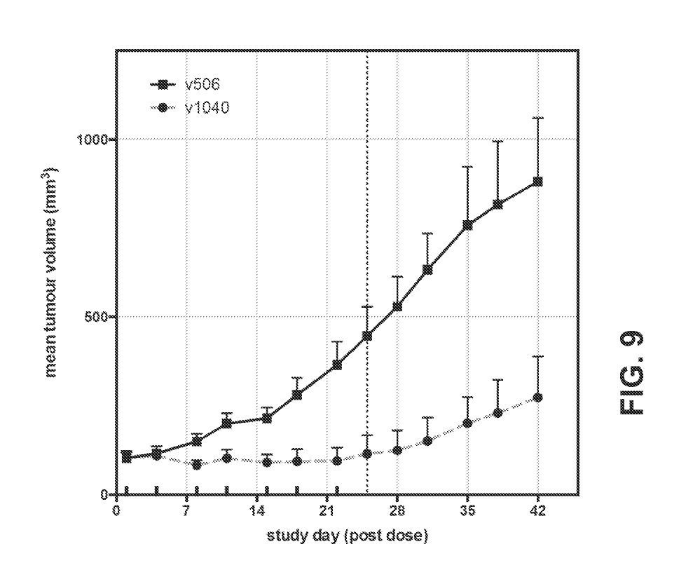

The results are shown in FIG. 9, where the vertical hashed line indicates the last dose date at day 24. The monovalent anti-HER2 antibody (v1040) showed significantly better tumor growth inhibition than the trastuzumab analog (506) and demonstrated significantly increased time to tumor progression compared to the trastuzumab analog of 41 and 15 days, respectively. Consistent with the PK results the serum exposure of the OA-Trastuzumab antibody on study day 11 was lower than the Trastuzumab analogue with values of 107.3 and 190.5 microg/ml respectively (Table 9). The results suggest that v1040 may have utility in Trastuzumab and chemotherapeutic resistant metastatic breast cancer.

Table 9 provides data comparing measurements of efficacy of the monovalent anti-HER2 antibody and the FSA-tras control (v506). Table 9 shows that monovalent anti-HER2 antibody (v1040) is superior compared to FSA-tras (v506) in the reduction of mean tumour volume (TV mm³), the number of responders (TV>50% of control), the number of complete response (<10% baseline regression) the number showing zero residual disease (TV<20 mm³), the number of progressive tumors (tumor doubling), and in the mean time to tumor progression (time to doubling).

TABLE 9

| Day 25, n = 7 | v506 | v1040 |
|---|---|---|
| Mean TV (mm3) | 447 | 115 |
| (% change from Baseline) | (+335%) | (+11%) |
| T/C (tras) ratio | 1 | 0.26 |
| Responders | 0/7 | 5/7 |
| (TV <50% of control) | | |
| Complete response | 0/7 | 4/7 |
| (>10% baseline regression) | | |
| Zero residual disease | 0/7 | 1/7 |
| (TV <20 mm3) | | |
| Mean time to progression | 15 | 41 |
| (days required for doubling | | |
| from baseline) | | |
| Number of progressing tumors | 7/7 | 5/7 |
| Day 25 Serum exposure | 190.5 | 107.3 |
| (microg/ml) | | |

### Example 12

### Monovalent Anti-HER2 Antibody Shows Increased Volume of Distribution Compared to a Bivalent HER2 Antibody (FSA)

The pharmacokinetics (PK) of an exemplary monovalent anti-HER2 antibody (v1040, OA-tras) were examined and compared to that of the control bivalent anti-HER2 antibody (v506, trastuzumab analog). These studies were carried out as described below
Strain/gender: CD-1 Nude/male
Target body weight of animals at treatment: 0.025 kg
Number of animals: 12, n=3/timepoint
Body weight: Recorded on the day prior to treatment for calculation of the volume to be administered.
Clinical signs observation: Up to 2 h post-injection and then twice daily from Day 1 to Day 11.
Mice were dosed on Day 1 by an IV injection into the tail vein with the test article at a dose of 10 mg/kg. Blood samples, approximately 0.060 mL, were collected from the submandibular or saphenous vein at selected time points (3 animals per time points) up to 240 h post-dose as per Table 8 below. Pre-treatment serum samples (Pre-Rx) were obtained from a naïve animal. Blood samples were allowed to clot at room temperature for 15 to 30 minutes. Blood samples were centrifuged to obtain serum at 2700 rpm for 10 min at room temperature and the serum stored at −80° C. For the terminal bleed, blood was collected by cardiac puncture.

Serum concentrations were determined by ELISA. Briefly, HER2 was coated at 0.5 ug/ml in PBS, 25 ul/well in a HighBind 384 plate (Corning 3700) plate and incubated overnight at 4° C. Well were washed 3× with PBS-0.05% tween-20 and blocked with PBS containing 1% BSA, 80 μl/well for 1-2 h at RT. Dilution of antibody serum and standards were prepared PBS containing 1% BSA. Following blocking, the block was removed and the antibody dilutions were transferred to the wells. The ELISA plate was centrifuged 30 sec at 1000 g to remove bubbles and the plate was incubated at RT for 2 h. The plate was washed 3× with PBS-0.05% tween-20 and 25 μl/well of AP-conjugated goat anti-human IgG, Fc (Jackson ImmunoResearch) was added (at a 1:5000 dilution in PBS containing 1% BSA) and incubated 1 h at RT. The plate was washed 4× with PBS-0.05% tween-20 and 25 μl/well of AP substrate (1 tablet in 5.5 mL pNPP buffer) was added. Using the Perkin Elmer Envision reader, read OD at 405 nm at different time intervals (0-30 minutes). The reaction was stopped with addition of 5 μL of 3N NaOH before the OD405 reached 2.2. The plate was centrifuged for 2 minutes at 1000 g before performing the last reading.

Serum concentrations were analysed using the WinnonLin software version 5.3 to obtain PK parameters. Serum samples were analyzed in two set of multiple dilutions and results within the validated range were accepted and averaged. Serum concentration values below the Lower Limit of Quantification (LLOQ) following ELISA analysis, were considered as 0 for the calculation of the mean serum concentration. The LLOQ obtained from the ELISA assays was approximately 1.2 μg/mL.

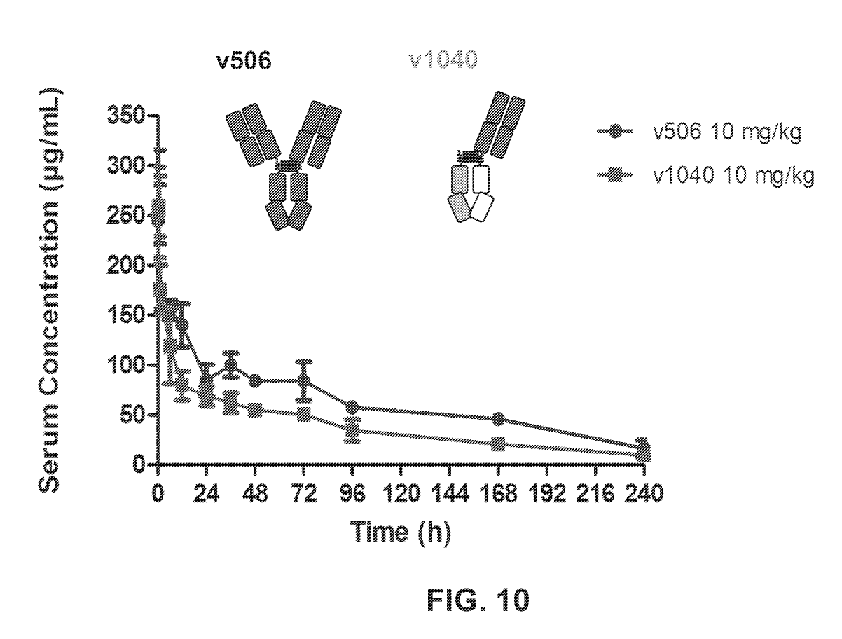

The results are shown in FIG. 10 and the PK parameters tested are shown in Table 10.

TABLE 10
PK Parameters

| | 506 | | v1040 | |
|---|---|---|---|---|
| Parameters | 10 mg/kg | % CV | 10 mg/kg | % CV |
| α (1/h) | 1.104 | 49.89 | 0.8065 | 32.93 |
| β (1/h) | 0.0089 | 23.29 | 0.0115 | 26.72 |
| k10 (1/h) | 0.0181 | 22.38 | 0.0329 | 21.75 |
| k12 (1/h) | 0.5515 | 59.20 | 0.5031 | 36.46 |
| k21 (1/h) | 0.5437 | 44.13 | 0.2820 | 32.37 |
| C0 | 292.5 | 12.57 | 301.4 | 8.52 |
| (μg/mL) | | | | |
| AUC | 16134 | 17.93 | 9158 | 19.49 |
| (μg · h/mL) | | | | |
| MRT (h) | 111.1 | 23.28 | 84.60 | 26.88 |
| Vc | 34.19 | 12.58 | 33.17 | 8.53 |
| (mL/kg) | | | | |
| Vp | 34.69 | 20.91 | 59.20 | 18.07 |
| (mL/kg) | | | | |
| CL | 0.620 | 17.95 | 1.092 | 19.51 |
| (mL/h/kg) | | | | |
| Vss | 68.88 | 8.96 | 92.37 | 11.38 |
| (mL/kg) | | | | |
| t½ α (h) | 0.628 | 49.85 | 0.8594 | 32.91 |
| t½ β (h) | 77.68 | 23.27 | 60.24 | 26.71 |

The results shown in FIG. 10 indicate that OA-tras has reasonable PK parameters for dosing in humans. Notably, OA-tras has a greater Vss (volume at steady state), indicating that the antibody is distributed in a greater volume and has a greater distribution into the tissues. Due to the increased tissue distribution of OA-tras, and OAAs in general, the antibodies may have therapeutic utility in treating disease in peripheral tissues where antibody concentration is limiting.

### Example 13

### Monovalent Anti-HER2 Antibody Shows Increased Blood-Brain-Barrier (BBB) Permeability Compared to FSA In Vitro

This experiment was performed in order to test the ability of an exemplary monovalent anti-HER2 antibody variant v1040 to pass through an in vitro BBB model. The in vitro BBB model used is described in detail in Garberg, M. Ball, N. Borg, R. Cecchelli, L. Fenart, R. D. Hurst, T. Lindmark, A. Mabondzo, J. E. Nilsson, T. J. Raub, D. Stanimirovic, T. Terasaki, J. O. Oberg, and T. Osterberg. In vitro models for the blood-brain barrier. Toxicol. in Vitro 19:299-334 (2005). This model used SV-ARBEC rat brain endothelial cells prepared as described in Garberg et al, supra.

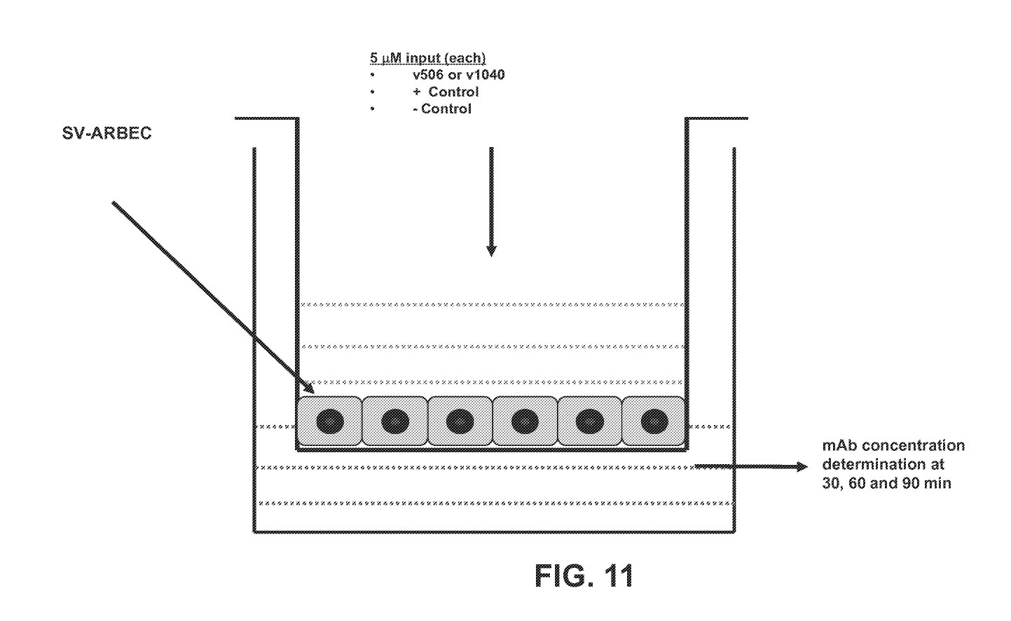

The experimental design of the assay is shown in FIG. 11. 80,000 rat brain endothelial cells (SV-ARBEC) were plated on a rat-tail collagen-coated 0.83 cm2 Falcon cell insert, 1 μm pore size in 1 mL SV-ARBEC feeding media without phenol red in a 12 well tissue culture plate. The bottom chamber contained 2 mL of 50:50 (v/v) mixture of SV-ARBEC media without phenol red and rat astrocyte-conditioned media. Transport experiments were performed in triplicate using transport buffer (10 mM HEPES, 5 mM MgCl₂, and 0.01% BSA in phosphate buffered saline, pH 7.4; 1 ml upper chamber and 2 ml bottom chamber) in ‘multiplexed’ fashion by adding: the test antibody; a positive control antibody (79 kDa VHH mouse Fc fusion, A20.1 VHH) with a known ability to transcytose; and a non-specific negative control antibody fragment (17 kDa VHH).

The test articles used in the assay and their size are described in Table 11 below.

TABLE 11

| | | Size |
|---|---|---|
| Variant | Target | (kDa) |
| v506, full-size bivalent, Trastuzumab | Human HER2 | 145 |
| v1040, OA-tras | Human HER2 | 98 |
| +Control | Specific Transcytosis | 79 |
| Control (A20.1VHH) | Non-specific | 17 |

The input concentration of each antibody was 5 μM and transcytosis for all antibodies was quantitated using the multiple reaction monitoring (MRM) method (Haqqani et al., 2008, Haqqani et al., 2008, 2013) by determining the antibody concentration of 1 l aliquot from the bottom chamber at 30, 60 and 90 min (followed by the replacement of 100 l of transport buffer into the bottom chamber after each aliquot collection).

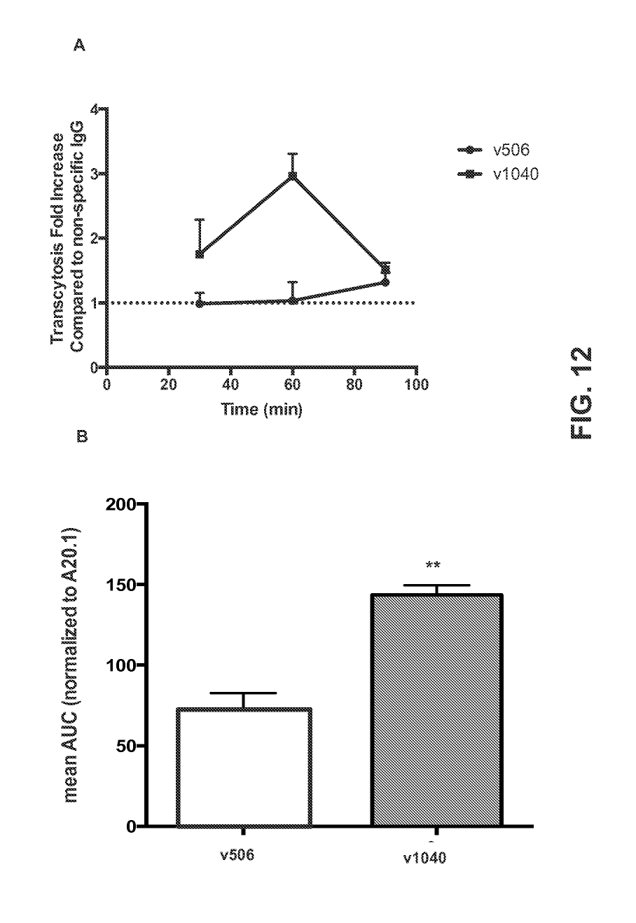

Briefly, MRM detects and quantitates peptide specific antibody fragments using previously described LCMS methods (Haqqani et al., 2008, Haqqani et al., 2008, 2013). Standard curves were used to calculate the concentration of each MRM peptide in the sample. The results of this assay are shown in FIG. 12. FIG. 12A shows the mean fold increase in transcytosis of v506 and v1040 compared to non-specific IgG at 30, 60 and 90 minutes. FIG. 12B shows the mean area under the curve (AUC) for both the bivalent and monovalent antibodies from all three replicates, AUC was calculated after normalization to the non-specific IgG control.

The results shown in FIG. 12B demonstrate that the monovalent anti-HER2 antibody shows a statistically significant 1.8-fold higher level of BBB transcytosis compared to the FSA.

### Example 14

### Increased In Vitro BBB Permeability of Monovalent Anti-HER2 Antibody is not Related to Molecular Weight

In order to determine the effect of molecular weight on the in vitro BBB permeability of test antibodies, the performance of variants 506 (control trastuzumab analog), 4182 (OA-pert), v6247 (v1040 conjugated to DM-1), v6248 (4182 conjugated to DM-1) and v630 (a monovalent anti-HER2 antibody based on trastuzumab where the antigen-binding domain is an scFv derived from trastuzumab, see additional description below) were compared to that of v1040.

V630 is a monovalent anti-HER2 antibody, where the HER2 binding domain is an scFv derived from trastuzumab, and the Fc region is a heterodimer having the mutations L351Y_S400E_F405A_Y407V in Chain A, and T366I_N390R_K392M_T394W in Chain B; v630 binds to domain 4 of HER2.

The assay was carried out as described in Example 13. The results are shown in Table 12 below.

TABLE 12

| | | | In vitro |
|---|---|---|---|
| | | Molecular | Transcytosis |
| | | Weight | (Mean normal- |
| Variant | Composition | (kDa) | ized AUC) |
| v506 | FSA-Trastuzumab_Fab | 145.6 | 54-71 |
| v1040 | OA-Trastuzumab_Fab | 98.9 | 143 |
| v4182 | OA-Pertuzumab_Fab | 98.8 | 120 |
| V6247 | OA-Trastuzumab_Fab- | 98.9 | 82 |
| (v1040-DM1) | ADC | | |
| V6248 | OA-Pertuzumab_Fab- | 98.8 | 92 |
| (4182-DM1) | ADC | | |
| v630 | OA-Trastuzumab_scFv- | 78.6 | 33 |
| | Fc | | |

These results demonstrate that one-armed (monovalent) antibodies composed of Fabs (either based on Trastuzumab or pertuzumab) have superior in vitro BBB permeability compared to full size antibodies. In addition, the mass of the antibodies appears to have no correlation to BBB permeability. Furthermore the conjugation of DM1 on the OAA reduced in vitro BBB permeability of the antibodies.

### Example 15

### Monovalent Anti-HER2 Antibody Shows Increased Brain and Lung Distribution Compared to Full Size Antibody

The ability of a monovalent anti-HER2 antibody, OA-tras (v1040), to distribute to the brain and lung was compared to full-size anti-HER2 antibody (v506, trastuzumab analog) using ex vivo imaging.

Female athymic nude mice were inoculated with a suspension of SKOV3 tumor cells subcutaneously. Tumors were monitored until they reached a tumor volume of ˜2000 mm3; animals were then randomized into 2 treatment groups: fluorescently labeled anti-HER2 full size antibody v506 or fluorescently labeled monovalent anti-HER2 antibody v1040. Both antibodies were fluorescently labeled with Cy5.5 for imaging, and both antibodies had a similar dye to protein ratio of ˜1.5:1. A single animal was dosed with each antibody at 10 mg/kg IV. 24 hours after injection the animals were anesthetized and had an intracardiac perfusion with heparinized saline. Following perfusion the brain and lungs were removed and optically imaged to determine antibody distribution.

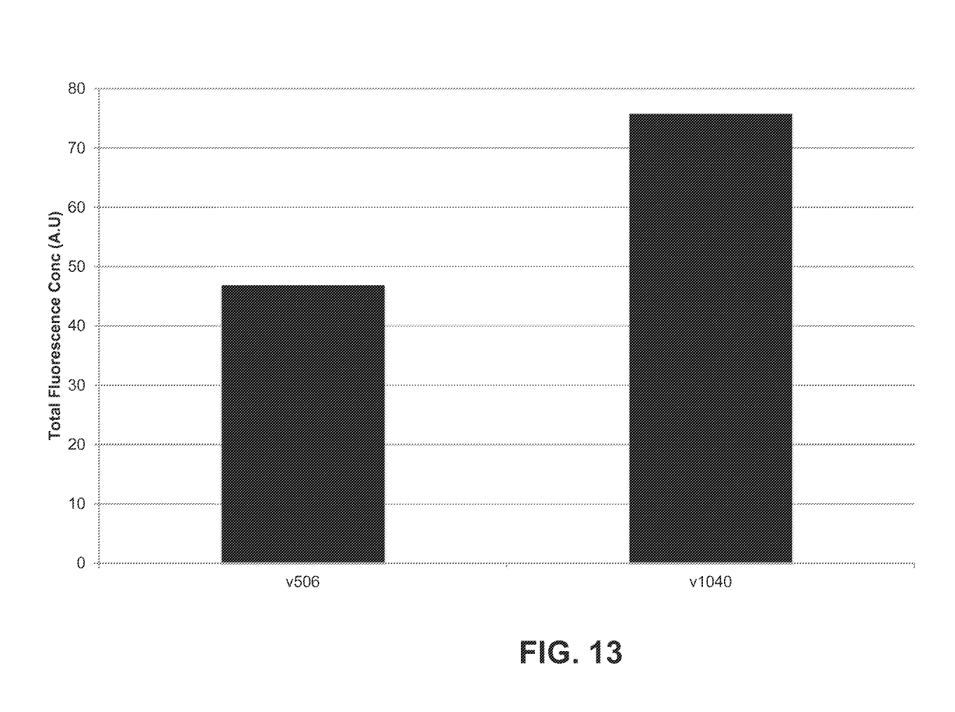

The results are shown in FIGS. 13 (brain) and 14 (lung). FIG. 13 demonstrates that v1040 has 1.6 fold greater brain distribution compared to v506. The superior brain distribution of the monovalent anti-HER2 antibody compared to FSA in brain may provide a therapeutic advantage in treating brain metastasis.

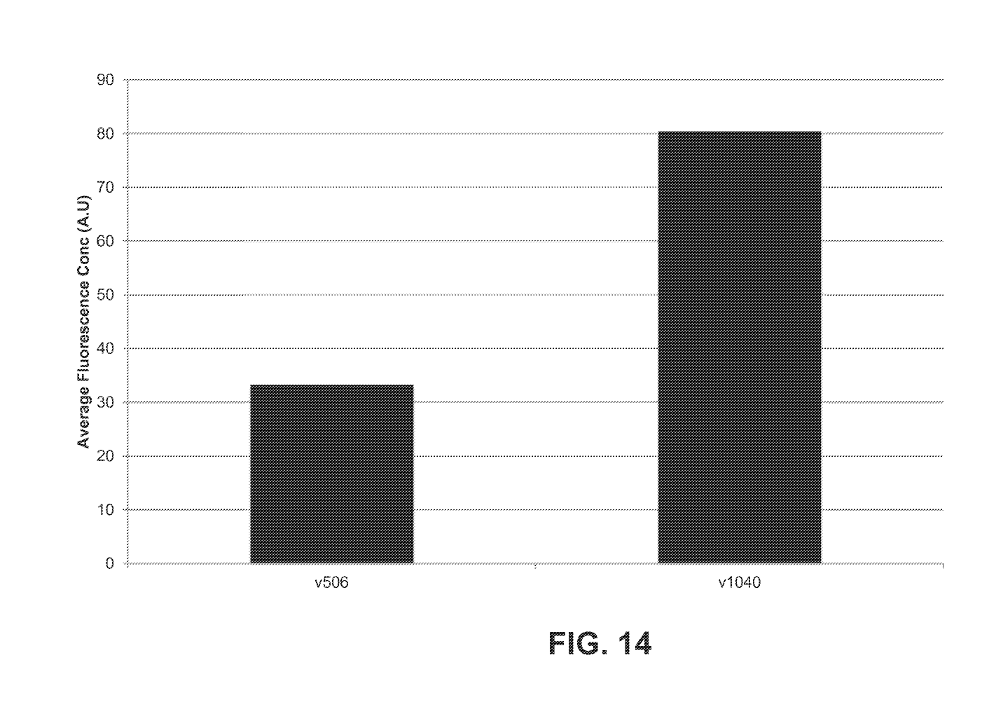

FIG. 14 demonstrates that the v1040 has 2.4 fold greater lung distribution compared to v506. The superior lung distribution of the monovalent anti-HER2 antibody compared to FSA may provide a therapeutic advantage in treating lung metastasis.

### Example 16

### Monovalent Anti-HER2 Antibody Shows Decreased Spontaneous Metastatic Lung Disease in a Trastuzumab Resistant PDX Model Compared to a T-DM1 Analog

The ability of a monovalent anti-HER2 antibody to prevent spontaneous lung metastasis was compared to buffer control and T-DM1 analog (v6246) in the trastuzumab resistant primary breast cancer xenograft model HBCx-13b. HBCx-13b is a HER2 3+, estrogen receptor negative, metastatic breast cancer that is innately resistant to Trastuzumab in nude mice.

Female athymic nude mice were inoculated subcutaneously with a 20 mm³ tumor fragment of HBCx-13b patient derived breast cancer serially passaged in mice. HBCx-13b is a HER3+, estrogen receptor negative, metastatic breast cancer that is resistant to Trastuzumab in nude mice. Tumors were then monitored until they reached an average volume of 150 mm³. Animals were then randomized into 3 treatment groups: vehicle control, T-DM1 analog (v6246) and afucosylated monovalent anti-HER2 antibody (OA-HER2-afuco, v7188) with either eight or nine animals in each group. v7188 is an afucosylated version of v1040, and used here as another example of a monovalent anti-HER2 antibody. The afucosylated version of v1040 was prepared using the same transient CHO expression system and protein A and size exclusion chromatography purification procedure described Example 1, but with the addition of an extra clone encoding a GDP-6-deoxy-D-lyxo-4-hexulose reductase (RMD) to 15% of the total DNA transfected.

Dosing was as follows:
A. Vehicle control was dosed intravenously with 5 ml/kg of formulation buffer twice per week to study day 39.
B. T-DM1 analog was dosed intravenously with 3 mg/kg on study day 0, 15, 33, and 43.
C. OA-HER2 afuco (v7188) was dosed intravenously with 10 mg/kg twice per week to study day 39.

On study day 43 four animals from each group were sacrificed and the lungs and tumors collected and stored for metastasis quantification by snap freezing. Metastases in the lungs were quantified by PCR of specific human Alu sequences as previously described (Scheuer W et al. 2009. Cancer Res 69:9330-9336). HBCx-13b frozen tumor lysate was used to generate a standard curve.

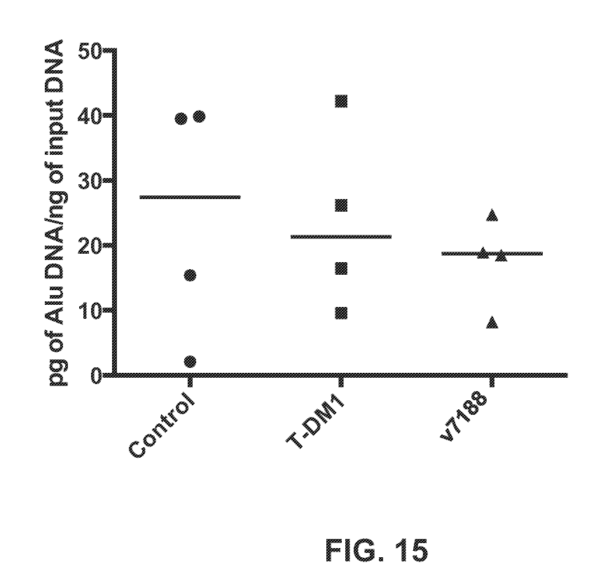

As shown in Table 13, HBCx-13b tumors were found to spontaneously metastasize to the lung in buffer-treated mice and had a median value of 27.4 pg of human DNA/ng input DNA (n=4). FIG. 15 shows that mice treated with T-DM1 analog or OA-HER2-afuco (v7188) had a median value of 21.3 and 18.7 pg of human DNA/ng input DNA, respectively (n=4). The OA-HER2-afuco antibody showed a trend towards reduced lung metastasis compared to control and T-DM1 analog. This result indicates that OA-HER2-afuco antibody may be efficacious in reducing lung metastases.

TABLE 13
PCR Quantification of human DNA indicating metastatic
disease in mice bearing HBCx-13b tumors.

| | | pg of huAluDNA/ng of |
|---|---|---|
| Treatment | Animal # | input DNA in lung |
| Control | 27 | 39.8 |
| | 57 | 39.5 |
| | 102 | 2.1 |
| | 204 | 15.4 |
| T-DM1 analog | 23 | 42.2 |
| | 53 | 16.5 |
| | 153 | 9.6 |
| | 167 | 26.1 |
| OA-HER2 -afuco | 24 | 18.9 |
| | 42 | 8.2 |
| | 145 | 18.5 |
| | 203 | 24.7 |

### Example 17

### Preparation of Additional OA Anti-HER2 Antibodies

The following OA antibodies were prepared as additional examples of OA anti-HER2 antibodies suitable for use according to the methods described herein. v1041, v630, and v878 have been previously described and characterized in International Patent Publication No. WO 2013/166604.

OA Antibodies Derived from Trastuzumab

v1041—a monovalent anti-HER2 antibody, where the HER2 binding domain is a Fab derived from trastuzumab on chain B, and the Fc region is a heterodimer having the mutations T350V_L351Y_F405A_Y407V in Chain A, T350V_T366L_K392L_T394W in Chain B; and the hinge region having the mutation C226S (EU numbering) in Chain A; the antigen binding domain binds to domain 4 of HER2.

v630—a monovalent anti-Her2 antibody, where the HER2 binding domain is an scFv derived from trastuzumab on Chain A, and the Fc region is a heterodimer having the mutations L351Y_S400E_F405A_Y407V in Chain A, T366I_N390R_K392M_T394W in Chain B; and the hinge region having the mutation C226S (EU numbering) in both chains; the antigen binding domain binds to domain 4 of HER2.

OA Antibody Derived from Antibody B1D2

v878—a monovalent anti-Her2 antibody, where the HER2 binding domain is a scFv on Chain A derived from the antibody B1D2 (generated from a known Her2/neu binding Ab (Schier R. et al. (1995) In vitro and in vivo characterization of a human anti-c-erbB-2 single-chain Fv isolated from a filamentous phage antibody library. Immunotechnology 1, 73)), and the Fc region is a heterodimer having the mutations L351Y_F405A_Y407V in Chain A, T366L_K392M_T394W in Chain B; and the hinge region having the mutation C226S (EU numbering) in both chains. The antigen binding domain binds to domain 1 of HER2.

OA Antibodies with Affinity-Improved Antigen-Binding Domains

v4442: a monovalent anti-HER2 antibody, where the HER2 binding domain is an affinity-improved Fab derived from pertuzumab on Chain A, having the mutations T30A on the heavy and Y96F on the light chain (Kabat numbering), the Fc region is a heterodimer having the mutations T350V_L351Y_F405A_Y407V in Chain A, and T350V_T366L_K392L_T394W in Chain B, and the hinge region having the mutation C226S (EU numbering) in Chain B; the antigen binding domain binds to domain 2 of HER2.

v4443: a monovalent anti-HER2 antibody, where the HER2 binding domain is an affinity-improved Fab derived from pertuzumab on Chain A, having the mutations T30A on the heavy chain and Y96A on the light chain (Kabat numbering), the Fc region is a heterodimer having the mutations T350V_L351Y_F405A_Y407V in Chain A, and T350V_T366L_K392L_T394W in Chain B, and the hinge region having the mutation C226S (EU numbering) in Chain B; the antigen binding domain binds to domain 2 of HER2.

v4444: a monovalent anti-HER2 antibody, where the HER2 binding domain is an affinity-improved Fab derived from pertuzumab on Chain A, having the mutations T30A and G57A on the heavy chain and Y96F on the light chain (Kabat numbering), the Fc region is a heterodimer having the mutations T350V_L351Y_F405A_Y407V in Chain A, and T350V_T366L_K392L_T394W in Chain B, and the hinge region having the mutation C226S (EU numbering) in Chain B; the antigen binding domain binds to domain 2 of HER2.

v4445: a monovalent anti-HER2 antibody, where the HER2 binding domain is an affinity-improved Fab derived from pertuzumab on Chain A, having the mutations T30A and G57A on the heavy chain and Y96A on the light chain (Kabat numbering), the Fc region is a heterodimer having the mutations T350V_L351Y_F405A_Y407V in Chain A, and T350V_T366L_K392L_T394W in Chain B, and the hinge region having the mutation C226S (EU numbering) in Chain B; the antigen binding domain binds to domain 2 of HER2.

For additional clarity, the amino acid residue numbers that have been modified in the Fab portions of the antibodies are identified in Table 14 below using both IMGT and Kabat numbering conventions.

TABLE 14
IMGT and Kabat numbering for modified residues in Fab region

| IMGT # | Kabat # |
|---|---|
| H_T35A | H_T30A |
| H_G64A | H_G56A |
| L_Y116A/F | L_Y96A/F |

All variants were prepared, expressed and purified as described in Example 1.

### Example 18

### Determination of Biophysical Properties of Affinity-Improved OA Anti-HER2 Antibodies

The biophysical properties of affinity-improved OA anti-HER2 antibodies 4442, 4443, 4444, and 4445 were assessed. The biophysical properties assessed were thermal stability and target binding affinity.

Thermal Stability

Thermal stability was assessed by differential scanning calorimetry as follows. Differential scanning calorimetry (DSC) was performed on SEC purified variants to evaluate thermodynamic stability of the molecule. The OA variants 4442, 4443, 4444, and 4445 were compared to the OA variant 4182, which is the OA variant with a wild-type pertuzumab Fab.

DSC experiments were carried out using a GE or MicroCal VP-Capillary instrument. The proteins were buffer-exchanged into PBS (pH 7.4) and diluted to 0.3 to 0.7 mg/mL with 0.137 mL loaded into the sample cell and measured with a scan rate of 1° C./min from 20 to 100° C. Data was analyzed using the Origin software (GE Healthcare) with the PBS buffer background subtracted.

The DSC results as indicated by melting temperature (Tm) are shown in Table 15 below.

Target Binding Affinity

The ability of the affinity-improved variants to bind to HER2 was assessed by surface plasmon resonance (SPR) as follows.

SPR was performed on SEC purified variants to evaluate the affinity of the variants for the HER2 extracellular domain. All SPR assays were carried out using a Biacore T200 instrument (GE Healthcare (Canada) Ltd. (Mississauga, ON)) with 1×PBS running buffer (1×PBS buffer with 0.05% Tween 20 with 0.5 M EDTA stock solution added to 3.4 mM final concentration) at 37° C. for trastuzumab, and at 25° C. for pertuzumab. Anti-human Fc surfaces were generated with a CM5 sensorchip using the condition described by the standard immobilization wizard template using the option to aim for immobilization level set to 2000 RU. All 4 flowcells (FC) were immobilized with a similar amount of anti-human Fc which was diluted in 10 mM NaOAc pH 4.5 at 5 to 10 ug/ml. The OA variant 6449 (binding trastuzumab) was compared to v1040, which is the OA variant with a wild-type (wt) trastuzumab Fab. The OA variants 4442, 4443, 4444, and 4445 were compared to the OA variant 4182, which is the OA variant with a wild-type pertuzumab Fab. Variants were injected at 0.5 to 2 μg/ml for 60 s at a flow rate of 10 μl/min on FC2, FC3 or FC4. FC1 was never used to capture variants and left as a blank control surface.

Kinetic parameters of Her2 binding to captured variants was determined using single cycle kinetics (SCK) and derived from a 1:1 model using the Biacore T200 evaluation software. Her2 was injected for 180 s at a flow rate of 50 μl/min, at a concentration series of 0.74, 2.22, 6.66, 20 and 60 nM; and dissociation time of 1800 s. All measurements were performed at least in duplicates.

The measured affinity of the OA antibodies (indicated by K_{D}) tested is also shown in Table 15 below.

TABLE 15
Summary of thermal stability and target affinity measurements

| | | Tm | | | |
|---|---|---|---|---|---|
| | | (difference | | STEDEV | |
| | | compared to | KD | KD | KD_wt/ |
| WT Fab | Variant | wt) | (M) | (M) | KD_var |
| Pertuzumab | 4442 | −1.3 | 2.65E−9 | 8.62E−10 | 5.2 |
| | 4443 | −1.1 | 1.70E−09 | 3.95E−10 | 8.5 |
| | 4444 | 0.9 | 2.86E−09 | 7.52E−10 | 4.7 |
| | 4445 | 1 | 2.18E−09 | 3.86E−10 | 6.5 |
| | 4182 | 0 | 1.49E−08 | 4.20E−09 | 1 |
| | (wt) | | | | |

The data shown in Table 15 indicates that the thermal stability of the affinity-improved variants is very similar to that of the wild-type control antibodies. The affinity-improved variant 6449 shows an increase in affinity of about 1.7-fold over the wild-type control. The affinity-improved variants 4442-4445 show an increase in affinity ranging from 4.7- to 8.5-fold over the wild-type control.

### Example 19

### Determination of the Ability of OA Anti-HER2 Affinity-Matured Variants to Bind to Cells Expressing HER2

The ability of the OA anti-HER2 affinity matured variants to bind to SKOV3 cells was assessed by FACS as described in Example 3. Only the variants with affinity-improved pertuzumab Fab were assessed here, and all variants were directly labeled using the AlexaFluor® 488 Protein Labeling Kit (Invitrogen, cat. no. A10235), according to the manufacturer's instructions.

The results are shown in Table 16 below and demonstrate the affinity-improved OA anti-HER2 variants tested here are able to bind to HER2 expressed on the surface of SKOV3 cells.

TABLE 16
Binding data in SKOV3 cells

| WT Fab | Variant | Bmax | KD (M) |
|---|---|---|---|
| Pertuzumab | 4442 | 12087 | 8.8E−09 |
| | 4443 | 10715 | 9.3E−09 |
| | 4444 | 11208 | 1.3E−08 |
| | 4445 | 10243 | 7.4E−09 |
| | 4182 | 9469 | 9.7E−09 |
| | (wt) | | |

### Example 20

### Ability of OA Anti-HER2 Affinity-Improved Variants to Internalize in HER2-Expressing Cells

The ability of the OA anti-HER2 variants with affinity-improved pertuzumab Fabs to internalize was assessed in SKOV3 cells. The assay was carried out as described in Example 6, except that each antibody was tested individually.

The results are shown in Table 17, and indicate that the OA anti-HER2 variants tested were able to internalize in SKOV3 cells to a similar degree as the control variant 4182.

TABLE 17
Internalization in SKOV3 cells

| WT Fab | Variant | Internal MFI |
|---|---|---|
| Pertuzumab | 4442 | 5482 |
| | 4443 | 5253 |
| | 4444 | 5212 |
| | 4445 | 4342 |
| | 4182 | 6268 |
| | (wt) | |

### Example 21

### Ability of Monovalent Anti-her2 Antibodies and Combinations to Mediate ADCC in HER2+ Cells

The following experiment was performed in order to assess the ability of monovalent anti-HER2 antibodies and combinations to mediate ADCC in the HER2+ tumor cell lines, SKBr3 (HER2 3+), ZR-75-1 (HER2 2+, estrogen receptor positive, breast cancer), MCF7 (HER2 1+) and MDA-MB-231 (HER2 0/1+, triple negative breast cancer; TNBC). The assay was carried out as described in Example 7, except that for the ZR-75-1 PBMCs were used as effector cells at an E/T=30:1. The monovalent antibodies tested were 1040, 4182, and the combination of 1040 and 4182, with Herceptin™ as the FSA control. All antibodies tested had comparable levels of fucosylation (approximately 88%) of the Fc N-linked glycan, as measured by glycopeptide analysis and detection by nanoLC-MS (data not shown). Herceptin™ was purchased from Roche.

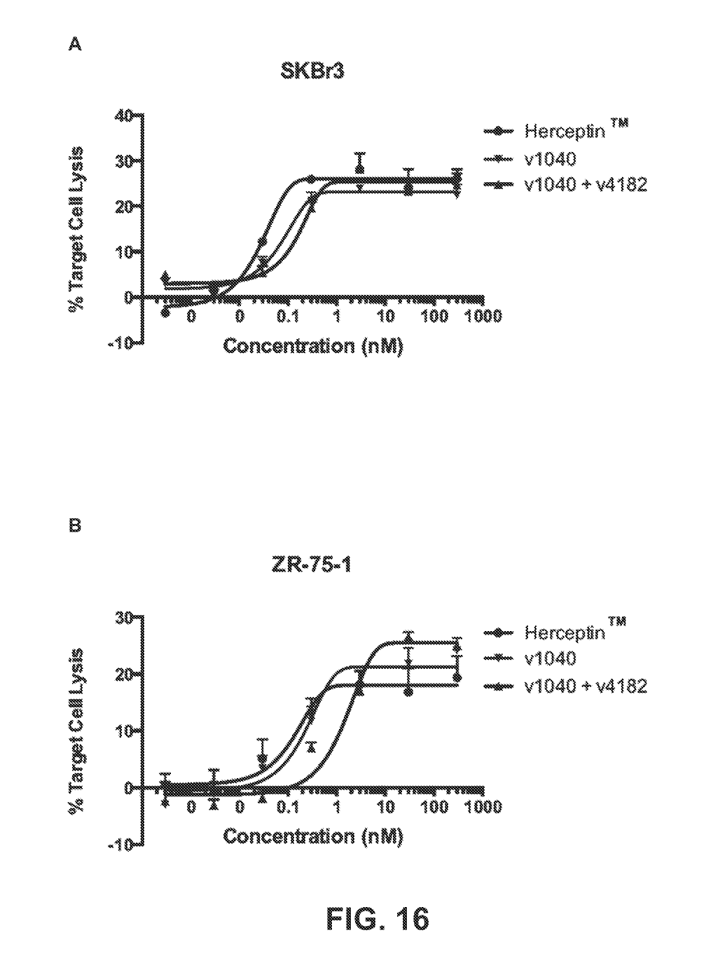

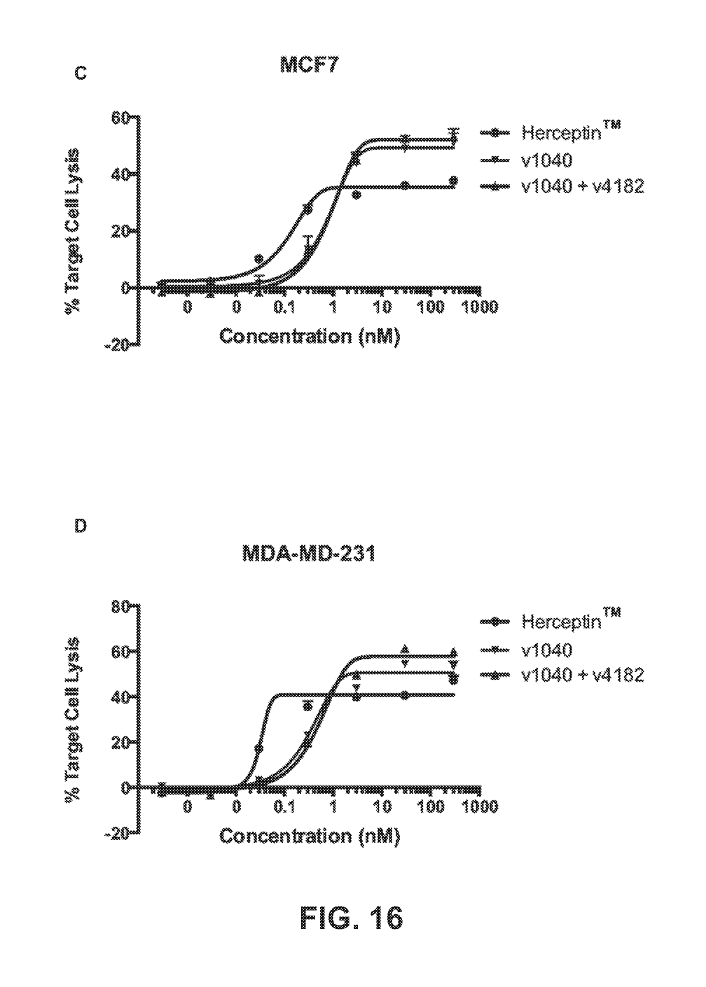

The results are shown in FIGS. 16A-D and Tables 18 to 21.

The results with SKBr3 HER2 3+ breast tumor cells are shown in FIG. 16A and Table 18 and indicate that the combination of two anti-HER2 OAAs can mediate equivalent maximum cell lysis by ADCC compared to an anti-HER2 FSA (Herceptin™) and the anti-HER2 OAA (v1040).

TABLE 18

| Antibody variant | % Max Cell Lysis |
|---|---|
| Herceptin | 26 |
| v1040 | 23 |
| v1040 + v4182 | 25 |

The results with ZR-75-1 HER2 2+ breast tumor cells are shown in FIG. 16B and Table 19, and indicate that the combination of two anti-HER2 OAAs can mediate approximately 1.4-fold greater maximum cell lysis by ADCC compared to an anti-HER2 FSA (commercial Herceptin) and approximately 1.2-fold greater compared to the anti-HER2 OAA (v1040).

TABLE 19

| Antibody variant | % Max Cell Lysis |
|---|---|
| Herceptin | 18 |
| v1040 | 21 |
| v1040 + v4182 | 26 |

The results with MCF7 HER2 1+ breast tumor cells are shown in FIG. 16C and Table 20, and indicate that the combination of two anti-HER2 OAAs can mediate approximately 1.5-fold greater maximum cell lysis by ADCC compared to an anti-HER2 FSA (commercial Herceptin) and equivalent ADCC compared to the anti-Her2 OAA (v1040).

TABLE 20

| Antibody variant | % Max Cell Lysis |
|---|---|
| Herceptin | 35 |
| v1040 | 49 |
| v1040 + v4182 | 52 |

The results with MDA-MB-231 HER2 0/1+TNBC tumor cells are shown in FIG. 16D and Table 21, and indicate that the combination of two anti-HER2 OAAs can mediate approximately 1.4-fold greater maximum cell lysis by ADCC compared to an anti-HER2 FSA (commercial Herceptin) and approximately 1.1-fold greater ADCC compared to the anti-HER2 OAA (v1040).

TABLE 21

| Antibody variant | % Max Cell Lysis |
|---|---|
| Herceptin | 41 |
| v1040 | 51 |
| v1040 + v4182 | 58 |

The results described here and in Example 7 show that combinations of two anti-HER2 OAAs are effective at mediating ADCC in HER2 3+, 2+, 1+ and 0/1+ breast and ovarian tumor cells with greater target cell lysis compared to an anti-HER2 FSA. The results also show a trend for greater ADCC with two anti-HER2 OAAs compared to one anti-HER2 OAA, in the HER2 2+ZR-75-1 and HER2 0/1+MDA-MB-231 breast tumor cells. This trend for increased ADCC with the OA combinations is consistent with the increased cell surface decoration shown in FIG. 2. Based on the cell-surface decoration (FIG. 2) and ADCC data in FIG. 5, it would be expected that the combination of two anti-HER2 OAAs would elicit greater maximum cell lysis compared to the an anti-HER2 FSA combination (v792+v4184) in HER2+ tumor cells.

Exemplary Variants, Clone Names, and SEQ ID NOS

TABLE 22

| SEQ ID NO | Variant name, sequence description, and type |
|---|---|
| 3 | 628 chain A DNA |
| 4 | 628 chain A amino acid |
| 5 | 628 chain B DNA |
| 6 | 628 chain B amino acid |
| 7 | 630 chain A DNA |
| 8 | 630 chain A amino acid |
| 9 | 630 chain B DNA |
| 10 | 630 chain B amino acid |
| 11 | 1040 chain A DNA |
| 12 | 1040 chain A amino acid |
| 13 | 1040 light chain DNA |
| 14 | 1040 light chain amino acid |
| 15 | 1040 chain B DNA |
| 16 | 1040 chain B amino acid |
| 17 | 1041 chain A DNA |
| 18 | 1041 chain A amino acid |
| 19 | 1041 light chain DNA |
| 20 | 1041 light chain amino acid |
| 21 | 1041 chain B DNA |
| 22 | 1041 chain B amino acid |
| 23 | 506 heavy chain DNA |
| 24 | 506 heavy chain amino acid |
| 25 | 506 light chain DNA |
| 26 | 506 light chain amino acid |
| 27 | 792 chain A Heavy DNA |
| 28 | 792 chain A Heavy amino acid |
| 29 | 792 light chain DNA |
| 30 | 792 light chain amino acid |
| 31 | 792 chain B Heavy DNA |
| 32 | 792 chain B Heavy amino acid |
| 33 | 871 chain A and B DNA |
| 34 | 871 Chain A and B amino acid |
| 35 | 878 chain A DNA |
| 36 | 878 chain A amino acid |
| 37 | 878 chain B DNA |
| 38 | 878 chain B amino acid |
| 39 | 4182 chain A DNA |
| 40 | 4182 chain A amino acid |
| 41 | 4182 chain B DNA |
| 42 | 4182 chain B amino acid |
| 43 | 4182 light chain DNA |
| 44 | 4182 light chain amino acid |
| 45 | 4183 chain A DNA |
| 46 | 4183 chain A amino acid |
| 47 | 4183 chain B DNA |
| 48 | 4183 chain B amino acid |
| 49 | 4183 light chain DNA |
| 50 | 4183 light chain amino acid |
| 51 | 4184 chain A DNA |
| 52 | 4184 chain A amino acid |
| 53 | 4184 chain B DNA |
| 54 | 4184 chain B amino acid |
| 55 | 4184 light chain DNA |
| 56 | 4184 light chain amino acid |

TABLE 23

| | H1 | H2 | L1 | L2 |
|---|---|---|---|---|
| Variant | (Clone) | (Clone) | (Clone) | (Clone) |
| 1040 | 4560 | 4553 | 4561 | n/a |
| 4182 | 4560 | 3057 | 1811 | n/a |
| 506 | 642 | 642 | 4561 | 4561 |
| 792 | 1011 | 1015 | 4561 | 4561 |
| 4184 | 3057 | 3041 | 1811 | 1811 |
| 1041 | 4558 | 4555 | 4561 | n/a |
| 630 | 719 | 716 | n/a | n/a |
| 878 | 1070 | 1039 | n/a | n/a |
| 4442 | 4560 | 3376 | 3383 | n/a |
| 4443 | 4560 | 3376 | 3382 | n/a |
| 4444 | 4560 | 3379 | 3383 | n/a |
| 4445 | 4560 | 3379 | 3382 | n/a |

TABLE 24

| SEQ ID NO | Clone | Desc. | Type |
|---|---|---|---|
| 57. | 642 | Full pr | Protein |
| 58. | 642 | Full | DNA |
| 59. | 642 | VH | Protein |
| 60. | 642 | VH | DNA |
| 61. | 642 | H1 | Protein |
| 62. | 642 | H1 | DNA |
| 63. | 642 | H3 | Protein |
| 64. | 642 | H3 | DNA |
| 65. | 642 | H2 | Protein |
| 66. | 642 | H2 | DNA |
| 67. | 642 | CH1 | Protein |
| 68. | 642 | CH1 | DNA |
| 69. | 642 | CH2 | Protein |
| 70. | 642 | CH2 | DNA |
| 71. | 642 | CH3 | Protein |
| 72. | 642 | CH3 | DNA |
| 73. | 716 | Full | Protein |
| 74. | 716 | Full | DNA |
| 75. | 716 | CH2 | Protein |
| 76. | 716 | CH2 | DNA |
| 77. | 716 | CH3 | Protein |
| 78. | 716 | CH3 | DNA |
| 79. | 1039 | Full | Protein |
| 80. | 1039 | Full | DNA |
| 81. | 1039 | CH2 | Protein |
| 82. | 1039 | CH2 | DNA |
| 83. | 1039 | CH3 | Protein |
| 84. | 1039 | CH3 | DNA |
| 85. | 1811 | Full | Protein |
| 86. | 1811 | Full | DNA |
| 87. | 1811 | VL | Protein |
| 88. | 1811 | VL | DNA |
| 89. | 1811 | L1 | Protein |
| 90. | 1811 | L1 | DNA |
| 91. | 1811 | L3 | Protein |
| 92. | 1811 | L3 | DNA |
| 93. | 1811 | L2 | Protein |
| 94. | 1811 | L2 | DNA |
| 95. | 1811 | CL | Protein |
| 96. | 1811 | CL | DNA |
| 97. | 1070 | Full | Protein |
| 98. | 1070 | Full | DNA |
| 99. | 1070 | VH | Protein |
| 100. | 1070 | VH | DNA |
| 101. | 1070 | H1 | Protein |
| 102. | 1070 | H1 | DNA |
| 103. | 1070 | H3 | Protein |
| 104. | 1070 | H3 | DNA |
| 105. | 1070 | H2 | Protein |
| 106. | 1070 | H2 | DNA |
| 107. | 1070 | VL | Protein |
| 108. | 1070 | VL | DNA |
| 109. | 1070 | L1 | Protein |
| 110. | 1070 | L1 | DNA |
| 111. | 1070 | L3 | Protein |
| 112. | 1070 | L3 | DNA |
| 113. | 1070 | L2 | Protein |
| 114. | 1070 | L2 | DNA |
| 115. | 1070 | CH2 | Protein |
| 116. | 1070 | CH2 | DNA |
| 117. | 1070 | CH3 | Protein |
| 118. | 1070 | CH3 | DNA |
| 119. | 3376 | Full | Protein |
| 120. | 3376 | Full | DNA |
| 121. | 3376 | VH | Protein |
| 122. | 3376 | VH | DNA |
| 123. | 3376 | H1 | Protein |
| 124. | 3376 | H1 | DNA |
| 125. | 3376 | H3 | Protein |
| 126. | 3376 | H3 | DNA |
| 127. | 3376 | H2 | Protein |
| 128. | 3376 | H2 | DNA |
| 129. | 3376 | CH1 | Protein |
| 130. | 3376 | CH1 | DNA |
| 131. | 3376 | CH2 | Protein |
| 132. | 3376 | CH2 | DNA |
| 133. | 3376 | CH3 | Protein |
| 134. | 3376 | CH3 | DNA |
| 135. | 3379 | Full | Protein |
| 136. | 3379 | Full | DNA |
| 137. | 3379 | VH | Protein |
| 138. | 3379 | VH | DNA |
| 139. | 3379 | H1 | Protein |
| 140. | 3379 | H1 | DNA |
| 141. | 3379 | H3 | Protein |
| 142. | 3379 | H3 | DNA |
| 143. | 3379 | H2 | Protein |
| 144. | 3379 | H2 | DNA |
| 145. | 3379 | CH1 | Protein |
| 146. | 3379 | CH1 | DNA |
| 147. | 3379 | CH2 | Protein |
| 148. | 3379 | CH2 | DNA |
| 149. | 3379 | CH3 | Protein |
| 150. | 3379 | CH3 | DNA |
| 151. | 3382 | Full | Protein |
| 152. | 3382 | Full | DNA |
| 153. | 3382 | VL | Protein |
| 154. | 3382 | VL | DNA |
| 155. | 3382 | L1 | Protein |
| 156. | 3382 | L1 | DNA |
| 157. | 3382 | L3 | Protein |
| 158. | 3382 | L3 | DNA |
| 159. | 3382 | L2 | Protein |
| 160. | 3382 | L2 | DNA |
| 161. | 3382 | CL | Protein |
| 162. | 3382 | CL | DNA |
| 163. | 3383 | Full | Protein |
| 164. | 3383 | Full | DNA |
| 165. | 3383 | VL | Protein |
| 166. | 3383 | VL | DNA |
| 167. | 3383 | L1 | Protein |
| 168. | 3383 | L1 | DNA |
| 169. | 3383 | L3 | Protein |
| 170. | 3383 | L3 | DNA |
| 171. | 3383 | L2 | Protein |
| 172. | 3383 | L2 | DNA |
| 173. | 3383 | CL | Protein |
| 174. | 3383 | CL | DNA |
| 175. | 4553 | Full | Protein |
| 176. | 4553 | Full | DNA |
| 177. | 4553 | VH | Protein |
| 178. | 4553 | VH | DNA |
| 179. | 4553 | H1 | Protein |
| 180. | 4553 | H1 | DNA |
| 181. | 4553 | H3 | Protein |
| 182. | 4553 | H3 | DNA |
| 183. | 4553 | H2 | Protein |
| 184. | 4553 | H2 | DNA |
| 185. | 4553 | CH1 | Protein |
| 186. | 4553 | CH1 | DNA |
| 187. | 4553 | CH2 | Protein |
| 188. | 4553 | CH2 | DNA |
| 189. | 4553 | CH3 | Protein |
| 190. | 4553 | CH3 | DNA |
| 191. | 4555 | Full | Protein |
| 192. | 4555 | Full | DNA |
| 193. | 4555 | VH | Protein |
| 194. | 4555 | VH | DNA |
| 195. | 4555 | H1 | Protein |
| 196. | 4555 | H1 | DNA |
| 197. | 4555 | H3 | Protein |
| 198. | 4555 | H3 | DNA |
| 199. | 4555 | H2 | Protein |
| 200. | 4555 | H2 | DNA |
| 201. | 4555 | CH1 | Protein |
| 202. | 4555 | CH1 | DNA |
| 203. | 4555 | CH2 | Protein |
| 204. | 4555 | CH2 | DNA |
| 205. | 4555 | CH3 | Protein |
| 206. | 4555 | CH3 | DNA |
| 207. | 4558 | Full | Protein |
| 208. | 4558 | Full | DNA |
| 209. | 4558 | CH2 | Protein |
| 210. | 4558 | CH2 | DNA |
| 211. | 4558 | CH3 | Protein |
| 212. | 4558 | CH3 | DNA |
| 213. | 719 | Full | Protein |
| 214. | 719 | Full | DNA |
| 215. | 719 | VL | Protein |
| 216. | 719 | VL | DNA |
| 217. | 719 | L1 | Protein |
| 218. | 719 | L1 | DNA |
| 219. | 719 | L3 | Protein |
| 220. | 719 | L3 | DNA |
| 221. | 719 | L2 | Protein |
| 222. | 719 | L2 | DNA |
| 223. | 719 | VH | Protein |
| 224. | 719 | VH | DNA |
| 225. | 719 | H1 | Protein |
| 226. | 719 | H1 | DNA |
| 227. | 719 | H3 | Protein |
| 228. | 719 | H3 | DNA |
| 229. | 719 | H2 | Protein |
| 230. | 719 | H2 | DNA |
| 231. | 719 | CH2 | Protein |
| 232. | 719 | CH2 | DNA |
| 233. | 719 | CH3 | Protein |
| 234. | 719 | CH3 | DNA |
| 235. | 4560 | Full | Protein |
| 236. | 4560 | Full | DNA |
| 237. | 4560 | CH2 | Protein |
| 238. | 4560 | CH2 | DNA |
| 239. | 4560 | CH3 | Protein |
| 240. | 4560 | CH3 | DNA |
| 241. | 4561 | Full | Protein |
| 242. | 4561 | Full | DNA |
| 243. | 4561 | VL | Protein |
| 244. | 4561 | VL | DNA |
| 245. | 4561 | L1 | Protein |
| 246. | 4561 | L1 | DNA |
| 247. | 4561 | L3 | Protein |
| 248. | 4561 | L3 | DNA |
| 249. | 4561 | L2 | Protein |
| 250. | 4561 | L2 | DNA |
| 251. | 4561 | CL | Protein |
| 252. | 4561 | CL | DNA |
| 253. | 3041 | Full | Protein |
| 254. | 3041 | Full | DNA |
| 255. | 3041 | VH | Protein |
| 256. | 3041 | VH | DNA |
| 257. | 3041 | H1 | Protein |
| 258. | 3041 | H1 | DNA |
| 259. | 3041 | H3 | Protein |
| 260. | 3041 | H3 | DNA |
| 261. | 3041 | H2 | Protein |
| 262. | 3041 | H2 | DNA |
| 263. | 3041 | CH1 | Protein |
| 264. | 3041 | CH1 | DNA |
| 265. | 3041 | CH2 | Protein |
| 266. | 3041 | CH2 | DNA |
| 267. | 3041 | CH3 | Protein |
| 268. | 3041 | CH3 | DNA |
| 269. | 3057 | Full | Protein |
| 270. | 3057 | Full | DNA |
| 271. | 3057 | VH | Protein |
| 272. | 3057 | VH | DNA |
| 273. | 3057 | H1 | Protein |
| 274. | 3057 | H1 | DNA |
| 275. | 3057 | H3 | Protein |
| 276. | 3057 | H3 | DNA |
| 277. | 3057 | H2 | Protein |
| 278. | 3057 | H2 | DNA |
| 279. | 3057 | CH1 | Protein |
| 280. | 3057 | CH1 | DNA |
| 281. | 3057 | CH2 | Protein |
| 282. | 3057 | CH2 | DNA |
| 283. | 3057 | CH3 | Protein |
| 284. | 3057 | CH3 | DNA |
| 285. | 1011 | Full | Protein |
| 286. | 1011 | Full | DNA |
| 287. | 1011 | VH | Protein |
| 288. | 1011 | VH | DNA |
| 289. | 1011 | H1 | Protein |
| 290. | 1011 | H1 | DNA |
| 291. | 1011 | H3 | Protein |
| 292. | 1011 | H3 | DNA |
| 293. | 1011 | H2 | Protein |
| 294. | 1011 | H2 | DNA |
| 295. | 1011 | CH1 | Protein |
| 296. | 1011 | CH1 | DNA |
| 297. | 1011 | CH2 | Protein |
| 298. | 1011 | CH2 | DNA |
| 299. | 1011 | CH3 | Protein |
| 300. | 1011 | CH3 | DNA |
| 301. | 1015 | Full | Protein |
| 302. | 1015 | Full | DNA |
| 303. | 1015 | VH | Protein |
| 304. | 1015 | VH | DNA |
| 305. | 1015 | H1 | Protein |
| 306. | 1015 | H1 | DNA |
| 307. | 1015 | H3 | Protein |
| 308. | 1015 | H3 | DNA |
| 309. | 1015 | H2 | Protein |
| 310. | 1015 | H2 | DNA |
| 311. | 1015 | CH1 | Protein |
| 312. | 1015 | CH1 | DNA |
| 313. | 1015 | CH2 | Protein |
| 314. | 1015 | CH2 | DNA |
| 315. | 1015 | CH3 | Protein |
| 316. | 1015 | CH3 | DNA |

1. A method of treating a subject comprising, administering an effective amount of a first monovalent antigen-binding construct or a combination of a first and a second monovalent antigen-binding construct to the subject,
a) wherein the first and second monovalent antigen-binding constructs each comprise an antigen-binding polypeptide construct and a dimeric Fc coupled, with or without a linker, to the antigen-binding polypeptide construct;
b) each antigen-binding polypeptide construct specifically binds a extracellular domain 2 (ECD2) of human epidermal growth factor receptor 2 (HER2), a ECD4 of HER2, or a ECD1 of HER2;
c) the first monovalent antigen-binding construct and the second monovalent antigen-binding construct bind to non-overlapping epitopes and do not compete with each other for binding to HER2,
d) wherein the first monovalent antigen-binding construct comprises v1040 and the second monovalent antigen-binding construct comprises v4182, and
e) wherein treating the subject is treating a HER2+ cancer that expresses HER2 at the 2+ level or lower as determined by immunohistochemistry (IHC).

2. A method of treating a subject comprising, administering an effective amount of a first monovalent antigen-binding construct or a combination of a first and a second monovalent antigen-binding construct to the subject,
a) wherein the first and second monovalent antigen-binding construct each comprise an antigen-binding polypeptide construct and a dimeric Fc coupled, with or without a linker, to the antigen-binding polypeptide construct;
b) each antigen-binding polypeptide construct specifically binds a extracellular domain 2 (ECD2) of human epidermal growth factor receptor 2 (HER2), a ECD4 of HER2, or a ECD1 of HER2; and
c) the first monovalent antigen-binding construct and the second monovalent antigen-binding construct bind to non-overlapping epitopes and do not compete with each other for binding to HER2.

3. The method of claim 1 or 2, wherein treating a subject is inhibiting growth of a HER2+ tumor, delaying progression of a HER2+ tumor, treating a HER2+ cancer or preventing a HER2+ cancer.

4. The method of claim 3, wherein the HER2+ tumor or cancer is selected from breast, ovarian, stomach, gastroesophageal junction, endometrial, salivary gland, head and neck, lung, brain, kidney, colon, colorectal, thyroid, pancreatic, prostate or bladder.

5. The method of claim 4, wherein the HER2+ tumor or cancer is selected from breast, ovarian, stomach, lung, or brain.

6. The method of claim 4, wherein the HER2+ tumor or cancer expresses HER2 at a 2+ level or lower, as determined by immunohistochemistry (IHC).

7. The method of claim 4, wherein the HER2+ tumor or cancer is an ovarian cancer that expresses HER2 at a 2+ or 3+ level, as determined by immunohistochemistry (IHC).

8. The method of claim 4, wherein the HER2+ tumor or cancer is a breast cancer.

9. The method of claim 8, wherein the breast cancer expresses HER2 at a 2+ level or lower, as determined by immunohistochemistry (IHC).

10. The method of claim 8, wherein the breast cancer is a trastuzumab-resistant breast cancer, a chemotherapy-resistant breast cancer, a triple-negative breast cancer, an estrogen receptor-negative breast cancer, or a estrogen receptor-positive breast cancer.

11. The method of claim 1 or 2, wherein the treating is treating or preventing a HER2+ metastatic cancer.

12. The method of claim 11, wherein the HER2+ metastatic cancer is a metastatic breast cancer, metastatic brain cancer or a metastatic lung cancer.

13. The method of claim 11, wherein the HER2+ cancer is an established primary and metastatic breast cancer, or a lung metastasis or brain metastasis of a breast cancer.

14. The method of claim 1, wherein the subject is a human.

15. The method of any of the above claims, the first monovalent antigen-binding construct comprising v1040 and the second monovalent antigen-binding construct comprising v4182.

16. The method of any one of claims 1 to 15, wherein the dimeric Fc is a heterodimeric Fc comprising at least two CH3 sequences and the dimerized CH3 sequences have a melting temperature (Tm) of about 68° C. or higher.

17. The method of any one of claims 1 to 16, wherein each monovalent antigen-binding construct selectively and/or specifically binds HER2 with a greater maximum binding (Bmax) as compared to a monospecific bivalent antigen-binding construct that specifically binds HER2, and wherein at a monovalent antigen-binding construct to target ratio of 1:1 the increase in Bmax relative to the monospecific bivalent antigen-binding construct is observed at a concentration greater than the observed equilibrium constant (KD) of the constructs up to saturating concentrations.

18. The method of any one of claims 2 to 17, wherein the antigen-binding polypeptide construct of the first monovalent antigen-binding construct comprises the v1040 antigen-binding polypeptide amino acid sequence and the antigen-binding polypeptide construct of the second monovalent antigen-binding construct comprises the v4182 antigen-binding polypeptide construct amino acid sequence.

19. The method of any one of claims 2 to 17, wherein the antigen-binding polypeptide construct of the first monovalent antigen-binding construct comprises an amino acid sequence at least 80%, 90%, 95%, 96%, 97%, 98%, or 99% identical to the v1040 antigen-binding polypeptide construct and the antigen-binding polypeptide construct of the second monovalent antigen-binding construct comprises an amino acid sequence at least 80%, 90%, 95%, 96%, 97%, 98%, or 99% identical to the v1040 antigen-binding polypeptide construct.

20. The method of any one of claims 2 to 17, wherein the first monovalent antigen-binding construct comprises a heterodimeric Fc of v1040 and the second monovalent antigen-binding construct comprises a heterodimeric Fc of v4182.

21. The method of any one of claims 2 to 17, wherein the first monovalent antigen-binding construct and the second monovalent antigen-binding construct are selected from v1041, v1041, v4182, v630, v878, v4442, v4443, v4444, and v4445.

22. The method of any one of claims 2 to 17, wherein the combination of first monovalent antigen-binding construct and second monovalent antigen-binding construct is v1040 and v4182.

23. The method of any one of claims 2 to 17, wherein only a first monovalent antigen-binding construct is administered and the first monovalent antigen-binding construct is selected from v1041, v1041, v4182, v630, v878, v4442, v4443, v4444, and v4445.

24. The method of claim 16, wherein each heterodimeric Fc
a. is a human Fc; and/or
b. is a human IgG1 Fc; and/or
c. comprises one or more modifications in at least one of the CH3 domains; and/or
d. comprises one or more modifications in at least one of the CH3 domains that promote the formation of a heterodimeric Fc with stability comparable to a wild-type homodimeric Fc; and/or
e. comprises one or more modifications in at least one of the CH3 domains as described in Table A2
f. further comprises at least one CH2 domain; and/or
g. further comprises at least one CH2 domain comprising one or more modifications; and/or
h. further comprises at least one CH2 domain comprising one or more modifications in at least one of the CH2 domains as described in Table A2; and/or
i. comprises one or more modifications to promote selective binding of Fc-gamma receptors.

25. The method of claim 24, wherein the dimerized CH3 domains have a melting temperature (Tm) of 68, 69, 70, 71, 72, 73, 74, 75, 76, 77, 77.5, 78, 79, 80, 81, 82, 83, 84, or 85° C. or higher.

26. The method of claim 24, wherein each heterodimeric Fc domain is fused to the antigen-binding polypeptide construct by a linker.

27. The method of claim 26, wherein the linker is a polypeptide linker.

28. The method of claim 26, wherein the linker comprises an IgG1 hinge region.

29. The method of any one of claims 2 to 28, wherein the first and/or second monovalent antigen-binding construct is conjugated to a drug.

30. The method of any one of claims 2 to 29, wherein the first and/or second monovalent antigen-binding construct is conjugated to maytansine (DM1).

31. The method of any one of claims 2 to 30, comprising administering the first and second monovalent antigen-binding constructs in a pharmaceutical composition.

32. The method of any one of claims 2 to 30, comprising administering the first and second monovalent antigen-binding constructs in a pharmaceutical composition comprising a buffer, an antioxidant, a low molecular weight molecule, a drug, a protein, an amino acid, a carbohydrate, a lipid, a chelating agent, a stabilizer, or an excipient.

33. The method of any one of claims 1 to 32, wherein the first and second monovalent antigen-binding constructs are co-administered.

34. The method of any one of claims 1 to 33, further comprising administering an additional agent.

35. The method of one of claims 1 to 34, wherein administering is orally or through injection.

36. A pharmaceutical composition for use in the method of any one of claims 1 to 35.

37. A method for
a. shrinking a tumor in a subject and/or
b. increasing overall survival in the subject wherein the subject has a tumor; and/or
c. treating a disorder characterized by HER2 expression in the subject; and/or
d. treating a disorder characterized by HER2 expression in the breast, colon, ovarian, gastro-intestinal, or brain tissue of the subject; and/or
e. treating a disorder characterized by HER2 expression in the subject wherein the subject is refractory or resistant to anti-HER2 treatments comprising trastuzumab and/or pertuzumab and/or trastuzumab emtansine (T-DM1); and/or
f. treating a cancer in the subject; and/or
g. treating a cancer in the subject wherein the subject is refractory to chemotherapy Standard of Care (SoC); and/or
h. treating a breast cancer in the subject;
the method comprising administering an effective amount of a first monovalent antigen-binding construct or a combination of a first and a second monovalent antigen-binding construct to the subject,
a) wherein the first and second monovalent antigen-binding construct each comprise at least one antigen-binding polypeptide construct and a dimeric Fc coupled, with or without a linker, to the antigen-binding polypeptide construct;
b) each antigen-binding polypeptide construct specifically binds a extracellular domain 2 (ECD2) of human epidermal growth factor receptor 2 (HER2), a ECD4 of HER2, or a ECD1 of HER2; and
c) the first monovalent antigen-binding construct and the second monovalent antigen-binding construct bind to non-overlapping epitopes and do not compete with each other for binding to HER2.

38. The method of claim 37, wherein the dimeric Fc comprises at least two CH3 domains and the dimerized CH3 domains have a melting temperature (Tm) of about 68° C. or higher.

39. The method of claim 37, wherein each monovalent antigen-binding construct selectively and/or specifically binds HER2 with a greater maximum binding (Bmax) as compared to monospecific bivalent antigen-binding construct that specifically binds HER2, and wherein at a construct to target ratio of 1:1 the increase in Bmax relative to the monospecific bivalent antigen-binding construct is observed at a concentration greater than the observed equilibrium constant (KD) of the constructs up to saturating concentrations.

40. The method of claim 37, wherein the first and second monovalent antigen-binding constructs are characterized by:
a. higher cell surface decoration in SKOV3 cells as determined by FACS and/or confocal microscopy when contacting cells with both constructs compared to contacting cells with each monovalent antigen-binding construct alone and/or an bivalent antigen-binding construct that specifically binds HER2; and/or
b. increased growth inhibition in BT-474 cells when contacting the cells with both constructs compared to contacting the cells with each monovalent antigen-binding construct alone; and/or
c. internalization of both monovalent antigen-binding constructs in SKOV3 cells when contacting the cells with both constructs; and/or
d. mediation of antibody-dependent cellular toxicity (ADCC) of SKOV3 cells when contacting the cells with both constructs; and/or
e. increased potency as measured by cell toxicity in SKOV3 and/or JIMT-1 cells when contacting the cells with both constructs compared to contacting the cell with each monovalent antigen-binding construct alone and/or
f. comparable potency as measured by cell toxicity in Herceptin resistant JIMT-1 cells when contacting the cells with both constructs compared to the cell with each monovalent antigen-binding construct alone.

41. A method of inhibiting growth of a HER2+ cancer cell, comprising contacting the HER2+ cancer cell with a first monovalent antigen-binding construct or a combination of a first and a second monovalent antigen-binding construct to the subject,
a. wherein the first and second monovalent antigen-binding construct each comprise at least one antigen-binding polypeptide construct and a dimeric Fc coupled, with or without a linker, to the antigen-binding polypeptide;
b. each antigen-binding polypeptide construct specifically binds a extracellular domain 2 (ECD2) of human epidermal growth factor receptor 2 (HER2), a ECD4 of HER2, or a ECD1 of HER2; and
c. the first monovalent antigen-binding construct and the second monovalent antigen-binding construct bind to non-overlapping epitopes and do not compete with each other for binding to HER2.

42. A method of killing HER2+ cancer cells, comprising contacting the HER2+ cancer cells with a first monovalent antigen-binding construct or a combination of a first and a second monovalent antigen-binding construct to the subject,
a. wherein the first and second monovalent antigen-binding construct each comprise at least one antigen-binding polypeptide construct and a dimeric Fc coupled, with or without a linker, to the antigen-binding polypeptide construct;
b. each antigen-binding polypeptide construct specifically binds a extracellular domain 2 (ECD2) of human epidermal growth factor receptor 2 (HER2), a ECD4 of HER2, or a ECD1 of HER2; and
c. the first monovalent antigen-binding construct and the second monovalent antigen-binding construct bind to non-overlapping epitopes and do not compete with each other for binding to HER2.

43. The method of claim 41 or 42, wherein the dimeric Fc comprises at least two CH3 domains and the dimerized CH3 domains have a melting temperature (Tm) of about 68° C. or higher.

44. The method of claim 41 or 42, wherein each monovalent antigen-binding construct selectively and/or specifically binds HER2 with a greater maximum binding (Bmax) as compared to an, monospecific bivalent antigen-binding construct that specifically binds HER2, and wherein at a construct to target ratio of 1:1 the increase in Bmax relative to the monospecific bivalent antigen-binding construct is observed at a concentration greater than the observed equilibrium constant (KD) of the constructs up to saturating concentrations.

45. The method of claim 42, wherein the first monovalent antigen-binding or the combination of first and second monovalent antigen-binding constructs mediate killing of HER2+ cancer cells by ADCC, ADCP, or CDC.

46. The method of claim 42, wherein the first monovalent antigen-binding or the combination of first and second monovalent antigen-binding constructs are conjugated to a drug.


SEQUENCE LISTING (317 sequences)

SEQ ID NO: 1 - protein, 17 aa, Homo sapiens
MLQNSAVLLLLVISASA

SEQ ID NO: 2 - protein, 22 aa, Artificial Sequence
  Description of Artificial Sequence Synthetic peptide
MPTWAWWLFLVLLLALWAPARG

SEQ ID NO: 3 - DNA, 1539 bp, Homo sapiens
gaattcgccaccatggccgtgatggctcctagaaccctggtgctgctgctgtctggagctctggctctgactcagacctgggctggagacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaaggtggttctggtggtggttctggtggtggttctggtggtggttctggtggtggttctggtgaagtgcagctggtggagtctgggggaggcttggtacagcctggcgggtccctgagactctcctgtgcagcctctggattcaacattaaagatacttatatccactgggtccggcaagctccagggaagggcctggagtgggtcgcacgtatttatcccacaaatggttacacacggtatgcggactctgtgaagggccgattcaccatctccgcagacacttccaagaacaccgcgtatctgcaaatgaacagtctgagagctgaggacacggccgtttattactgttcaagatggggcggagacggtttctacgctatggactactggggccaagggaccctggtcaccgtctcctcagccgccgagcccaagagcagcgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacatacccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacgaggatggcagcttcgccctggtgtccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 4 - protein, 510 aa, Homo sapiens
  misc_feature (30)..(510): Final protein product
EFATMAVMAPRTLVLLLSGALALTQTWAGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDEDGSFALVSKLTVDKSRWQQGNVFSCSVMHEALHNHYTQKSLSLSPGK

SEQ ID NO: 5 - DNA, 1539 bp, Homo sapiens
gaattcgccaccatggccgtgatggctcctagaaccctggtgctgctgctgtctggagctctggctctgactcagacctgggctggagacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaaggtggttctggtggtggttctggtggtggttctggtggtggttctggtggtggttctggtgaagtgcagctggtggagtctgggggaggcttggtacagcctggcgggtccctgagactctcctgtgcagcctctggattcaacattaaagatacttatatccactgggtccggcaagctccagggaagggcctggagtgggtcgcacgtatttatcccacaaatggttacacacggtatgcggactctgtgaagggccgattcaccatctccgcagacacttccaagaacaccgcgtatctgcaaatgaacagtctgagagctgaggacacggccgtttattactgttcaagatggggcggagacggtttctacgctatggactactggggccaagggaccctggtcaccgtctcctcagccgccgagcccaagagcagcgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgatctgtctggtgaaaggcttctatcctagtgatattgctgtggagtgggaatcaaatggacagccagagaacagatacatgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 6 - protein, 510 aa, Homo sapiens
  misc_feature (30)..(510): Final protein product
EFATMAVMAPRTLVLLLSGALALTQTWAGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLICLVKGFYPSDIAVEWESNGQPENRYMTWPPVLDSDGSFFLYSKLTVDKSRWQQGNVFSCSVMHEALHNHYTQKSLSLSPGK

SEQ ID NO: 7 - DNA, 1539 bp, Homo sapiens; identical to SEQ ID NO: 3

SEQ ID NO: 8 - protein, 510 aa, Homo sapiens; identical to SEQ ID NO: 4
  misc_feature (30)..(510): Final protein product

SEQ ID NO: 9 - DNA, 792 bp, Homo sapiens
gaattcgccaccatggccgtgatggctcctagaaccctggtgctgctgctgtctggagctctggctctgactcagacctgggctggagagcccaagagcagcgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgatctgtctggtgaaaggcttctatcctagtgatattgctgtggagtgggaatcaaatggacagccagagaacagatacatgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 10 - protein, 261 aa, Homo sapiens
  misc_feature (30)..(261): Final protein product
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLICLVKGFYPSDIAVEWESNGQPENRYMTWPPVLDSDGSFFLYSKLTVDKSRWQQGNVFSCSVMHEALHNHYTQKSLSLSPGK

SEQ ID NO: 11 - DNA, 1446 bp, Homo sapiens
gaattcgccaccatggccgtgatggctccaagaaccctggtcctgctgctgagtggggcactggctctgacacagacatgggccggggaagtccagctggtcgaaagcggaggaggactggtgcagccaggagggtctctgcgactgagttgcgccgcttcaggcttcaacatcaaggacacctacattcactgggtgcgccaggctcctggaaaaggcctggagtgggtggcacgaatctatccaaccaatggatacacacggtatgccgacagcgtgaagggccggttcaccattagcgcagatacttccaaaaacaccgcctacctgcagatgaacagcctgcgagccgaagataccgctgtgtactattgcagtcggtggggaggcgacggcttctacgctatggattattgggggcagggaacactggtcactgtgagctccgcatctactaaggggcctagtgtgtttccactggccccctctagtaaatccacatctgggggaactgcagccctgggatgtctggtgaaggactatttcccagagcccgtcacagtgagttggaactcaggcgccctgacttccggggtccatacctttcctgctgtgctgcagtcaagcggcctgtactctctgtcctctgtggtcacagtgccaagttcaagcctggggacccagacatatatctgcaacgtgaatcacaagccaagcaatactaaagtcgacaagaaagtggaacccaagagctgtgataaaactcatacctgcccaccttgtcctgcaccagagctgctgggaggaccatccgtgttcctgtttccacccaagcctaaagacaccctgatgatttccaggaccccagaagtcacatgcgtggtcgtggacgtgtctcacgaggaccccgaagtcaagttcaactggtacgtggatggcgtcgaggtgcataatgccaagacaaaacccagggaggaacagtacaactcaacatatcgcgtcgtgagcgtcctgactgtgctgcaccaggactggctgaacggcaaggagtataagtgcaaagtgagcaataaggctctgcccgcacctatcgagaaaaccattagcaaggctaaagggcagcctagagaaccacaggtctacgtgctgcctccaagcagggacgagctgacaaagaaccaggtctccctgctgtgtctggtgaaagggttctatcccagtgatatcgcagtggagtgggaatcaaatggacagcctgaaaacaattacctgacctggccccctgtgctggacagcgatggcagcttcttcctgtattccaagctgacagtggataaatctcggtggcagcagggcaacgtctttagttgttcagtgatgcatgaggccctgcacaatcattacacccagaagagcctgtccctgtctcccggcaaatgaggatcc

SEQ ID NO: 12 - protein, 479 aa, Homo sapiens
  misc_feature (30)..(479): Final protein product
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 13 - DNA, 738 bp, Homo sapiens
gaattcgccactatggccgtgatggcacctagaaccctggtcctgctgctgtccggggcactggcactgactcagacttgggctggggatattcagatgacccagtcccctagctccctgtccgcttctgtgggcgacagggtcactatcacctgccgcgcatctcaggatgtgaacaccgcagtcgcctggtaccagcagaagcctgggaaagctccaaagctgctgatctacagtgcatcattcctgtattcaggagtgcccagccggtttagcggcagcagatctggcaccgacttcacactgactatctctagtctgcagcctgaggattttgccacatactattgccagcagcactataccacaccccctactttcggccaggggaccaaagtggagatcaagcgaactgtggccgctccaagtgtcttcatttttccacccagcgacgaacagctgaaatccggcacagcttctgtggtctgtctgctgaacaacttctaccccagagaggccaaagtgcagtggaaggtcgataacgctctgcagagtggcaacagccaggagagcgtgacagaacaggactccaaagattctacttatagtctgtcaagcaccctgacactgagcaaggcagactacgaaaagcataaagtgtatgcctgtgaggtgacccatcaggggctgtcttctcccgtgaccaagtctttcaaccgaggcgaatgttgaggatcc

SEQ ID NO: 14 - protein, 243 aa, Homo sapiens
  misc_feature (30)..(243): Final protein product
EFATMAVMAPRTLVLLLSGALALTQTWAGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 792 bp, Homo sapiens
gaattcgccaccatggctgtgatggctccacgcaccctggtcctgctgctgtccggggcactggcactgactcagacttgggctggggaacctaaaagcagcgacaagacccacacatgccccccttgtccagctccagaactgctgggaggaccaagcgtgttcctgtttccacccaagcccaaagatacactgatgatcagccgaactcccgaggtcacctgcgtggtcgtggacgtgtcccacgaggaccccgaagtcaagttcaactggtacgtggacggcgtcgaagtgcataatgcaaagactaaaccacgggaggaacagtacaactctacatatagagtcgtgagtgtcctgactgtgctgcatcaggattggctgaacggcaaagagtataagtgcaaagtgtctaataaggccctgcctgctccaatcgagaaaactattagtaaggcaaaagggcagcccagggaacctcaggtctacgtgctgcctccaagtcgcgacgagctgaccaagaaccaggtctcactgctgtgtctggtgaaaggattctatccttccgatattgccgtggagtgggaatctaatggccagccagagaacaattacctgacctggccccctgtgctggacagcgatgggtccttctttctgtattcaaagctgacagtggacaaaagcagatggcagcagggaaacgtctttagctgttccgtgatgcacgaagccctgcacaatcattacacccagaagtctctgagtctgtcacctggcaaatgaggatcc

SEQ ID NO: 16 - protein, 261 aa, Homo sapiens
  misc_feature (30)..(261): Final protein product
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 17 - DNA, 1446 bp, Homo sapiens; identical to SEQ ID NO: 11

SEQ ID NO: 18 - protein, 479 aa, Homo sapiens; identical to SEQ ID NO: 12
  misc_feature (30)..(479): Final protein product

SEQ ID NO: 19 - DNA, 738 bp, Homo sapiens; identical to SEQ ID NO: 13

SEQ ID NO: 20 - protein, 243 aa, Homo sapiens; identical to SEQ ID NO: 14
  misc_feature (30)..(243): Final protein product

SEQ ID NO: 21 - DNA, 792 bp, Homo sapiens
gaattcgccaccatggccgtgatggcacctagaaccctggtcctgctgctgagcggggcactggcactgacacagacttgggctggggaacctaagagcagcgacaagactcacacctgcccaccttgtccagcaccagaactgctgggaggaccaagcgtgttcctgtttccacccaagcccaaagataccctgatgatcagccgaacacccgaagtgacttgcgtggtcgtggacgtgtcccacgaggaccccgaagtcaagttcaactggtacgtggacggcgtcgaagtgcataatgctaagacaaaaccacgggaggaacagtacaactctacttatagagtcgtgagtgtcctgaccgtgctgcatcaggattggctgaacggcaaagagtataagtgcaaagtgtctaataaggccctgcctgctccaatcgagaaaaccattagtaaggctaaagggcagcccagggaacctcaggtctacgtgtatcctccaagtcgcgacgagctgaccaagaaccaggtctcactgacatgtctggtgaaaggattttacccttccgatattgcagtggagtgggaatctaatggccagccagagaacaattataagaccacaccccctgtgctggacagcgatgggtccttcgcactggtctcaaagctgacagtggacaaaagcagatggcagcagggaaacgtctttagctgttccgtgatgcacgaagccctgcacaatcattacactcagaagtctctgagtctgtcacctggcaaatgaggatcc

SEQ ID NO: 22 - protein, 261 aa, Homo sapiens
  misc_feature (30)..(261): Final protein product
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 23 - DNA, 1446 bp, Homo sapiens
gaattcgccaccatggccgtgatggctcctagaaccctggtgctgctgctgtctggagctctggctctgactcagacctgggctggagaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 24 - protein, 479 aa, Homo sapiens
  misc_feature (30)..(479): Final protein product
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 738 bp, Homo sapiens
gaattcgccactatggctgtgatggcccctaggaccctggtgctgctgctgtccggagctctggctctgactcagacctgggctggagacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaagagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgaggatcc

SEQ ID NO: 26 - protein, 243 aa, Homo sapiens; identical to SEQ ID NO: 14
  misc_feature (30)..(243): Final protein product

SEQ ID NO: 27 - DNA, 1446 bp, Homo sapiens
gaattcgccaccatggccgtgatggctcctagaaccctggtgctgctgctgtctggagctctggctctgactcagacctgggctggagaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacgtgtacccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcgccctggtgtccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 28 - protein, 479 aa, Homo sapiens
  misc_feature (30)..(479): Final protein product
EFATMAVMAPRTLVLLLSGALALTQTWAGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 29 - DNA, 738 bp, Homo sapiens; identical to SEQ ID NO: 25

SEQ ID NO: 30 - protein, 243 aa, Homo sapiens; identical to SEQ ID NO: 14
  misc_feature (30)..(243): Final protein product

SEQ ID NO: 31 - DNA, 1446 bp, Homo sapiens
gaattcgccaccatggccgtgatggctcctagaaccctggtgctgctgctgtctggagctctggctctgactcagacctgggctggagaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacgtgctgccacccagcagagacgaactgaccaagaaccaggtgtccctgctgtgtctggtgaaaggcttctatcctagtgatattgctgtggagtgggaatcaaatggacagccagagaacaattacctgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaatgaggatcc

SEQ ID NO: 32 - protein, 479 aa, Homo sapiens; identical to SEQ ID NO: 12
  misc_feature (30)..(479): Final protein product

SEQ ID NO: 33 - DNA, 1563 bp, Homo sapiens
gaattcgccaccatggctgtgatggctcctagaacactggtcctgctgctgtccggggcactggcactgactcagacttgggccgggcaggtccagctggtgcagagcggggcagaggtcaagaaacccggagaaagtctgaagatctcatgcaaagggagtggatactcattcaccagctattggattgcctgggtgaggcagatgcctggcaaggggctggaatacatgggcctgatctatccaggggacagcgatacaaaatactccccctctttccagggccaggtcacaatttccgtggacaagagtgtctcaactgcttatctgcagtggagctccctgaaacctagcgattccgcagtgtacttttgtgccaggcacgacgtcgggtattgcacagatcgcacttgtgcaaagtggccagagtggctgggagtgtggggacagggaaccctggtcacagtgtctagtggaggaggaggctcaagcggaggaggctctggaggaggagggtctcagagtgtgctgactcagccaccttcagtcagcgcagctcctggacagaaggtgaccatctcctgctctggcagctctagtaacattggcaacaattacgtgagctggtatcagcagctgcctggcaccgccccaaagctgctgatctacgaccacacaaatcggcccgctggggtgcctgatagattcagtgggtcaaaaagcggaacctccgcttctctggcaattagcggctttcgctccgaggacgaagctgattactattgtgcatcttgggactacacactgagtggctgggtgttcggaggcgggactaagctgaccgtgctgggggcagccgaaccaaagtcaagcgataaaactcatacctgcccaccatgtcctgcaccagagctgctgggaggaccttccgtgttcctgtttcctccaaagccaaaagacaccctgatgatcagccgaacaccagaagtgacttgcgtggtcgtggacgtctcccacgaggaccccgaagtgaagtttaactggtacgtggatggcgtcgaggtgcataatgccaagaccaaaccccgagaggaacagtacaactcaacttatcgggtcgtgagcgtcctgaccgtgctgcaccaggactggctgaacgggaaagagtataagtgcaaagtgtctaataaggccctgcccgctcctatcgagaaaacaattagcaaggccaaaggccagccaagagaaccccaggtgtacactctgcccccttctagggacgagctgaccaagaaccaggtgagcctgacatgtctggtcaaaggattctatcccagtgatattgctgtggagtgggaatccaatggccagcctgaaaacaattacaagaccacaccacccgtgctggactccgatggatctttctttctgtattccaagctgactgtggataaatctcggtggcagcagggcaacgtgtttagttgttcagtcatgcatgaggccctgcacaatcattacacacagaagagcctgtccctgtctcccggcaaatgaggatcc

SEQ ID NO: 34 - protein, 518 aa, Homo sapiens
  misc_feature (30)..(518): Final protein product
EFATMAVMAPRTLVLLLSGALALTQTWAGQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSSGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1563 bp, Homo sapiens
gaattcgccaccatggccgtgatggcacctagaacactggtcctgctgctgagcggagccctggcactgacacagacttgggccggacaggtccagctggtgcagagcggggcagaggtcaagaaacccggagaaagtctgaagatctcatgcaaagggagtggatactcattcaccagctattggattgcctgggtgaggcagatgcctggcaaggggctggaatacatgggcctgatctatccaggggacagcgatacaaaatactccccctctttccagggccaggtcacaatttccgtggacaagagtgtctcaactgcctatctgcagtggagctccctgaaacctagcgattccgcagtgtacttttgtgccaggcacgacgtcgggtattgcacagatcgcacttgtgctaagtggccagagtggctgggagtgtggggacagggaaccctggtcacagtgtctagtggaggaggaggctcaagcggaggaggctctggaggaggagggtctcagagtgtgctgactcagccaccttcagtcagcgcagctcctggacagaaggtgaccatctcctgctctggcagctctagtaacattggcaacaattacgtgagctggtatcagcagctgcctggcaccgccccaaagctgctgatctacgaccacacaaatcggcccgctggggtgcctgatagattcagtgggtcaaaaagcggaacctccgcttctctggcaattagcggctttcgctccgaggacgaagctgattactattgtgcatcttgggactacacactgagtggctgggtgttcggaggcgggactaagctgaccgtgctgggggcagccgaaccaaagtcaagcgataaaactcatacctgcccaccatgtcctgcaccagagctgctgggaggaccttccgtgttcctgtttcctccaaagccaaaagacaccctgatgatcagccgaacaccagaagtgacttgcgtggtcgtggacgtctcccacgaggaccccgaagtgaagtttaactggtacgtggatggcgtcgaggtgcataatgccaagaccaaaccccgagaggaacagtacaactcaacttatcgggtcgtgagcgtcctgaccgtgctgcaccaggactggctgaacgggaaagagtataagtgcaaagtgtctaataaggccctgcccgctcctatcgagaaaacaattagcaaggcaaaaggccagccaagagaaccccaggtgtacacttatcccccttctagggacgagctgaccaagaaccaggtgagcctgacatgtctggtcaaaggattttaccccagtgatattgctgtggagtgggaatccaatggccagcctgaaaacaattataagaccacaccacccgtgctggactccgatggatctttcgctctggtgtccaagctgactgtcgataaatctcggtggcagcagggcaacgtgtttagttgttcagtcatgcatgaggcactgcacaatcattacacacagaagagcctgtccctgtctcccggcaaatgaggatcc

SEQ ID NO: 36 - protein, 518 aa, Homo sapiens [sequence omitted]

SEQ ID NO: 37 - DNA, 792 bp, Homo sapiens
gaattcgctacaatggccgtgatggcaccccgaacactggtcctgctgctgagcggcgcactggcactgacacagacttgggctggagaacctaagagctccgacaaaacccacacatgccccccttgtccagctccagaactgctgggaggaccatccgtgttcctgtttccacccaagcccaaagatacactgatgatctctcgaactcccgaggtcacctgcgtggtcgtggacgtcagtcacgaggaccccgaagtcaagttcaactggtacgtggacggcgtcgaagtgcataatgcaaagactaaaccacgggaggaacagtacaactcaacatatagagtcgtgagcgtcctgactgtgctgcatcaggattggctgaacggcaaggagtataagtgcaaagtgagcaataaggccctgcctgctccaatcgagaaaaccattagcaaggcaaaagggcagcccagggaacctcaggtgtacaccctgcctccaagccgcgacgagctgacaaagaaccaggtctccctgctgtgtctggtgaaaggattctatcctagtgatattgccgtggagtgggaatcaaatggccagccagagaacaattacatgacttggccccctgtgctggactctgatgggagtttctttctgtattccaagctgaccgtggacaaatctagatggcagcagggaaacgtcttttcttgtagtgtgatgcacgaagccctgcacaatcattacacacagaagtcactgagcctgtcccctggcaaatgaggatcc

SEQ ID NO: 38 - protein, 261 aa, Homo sapiens
  misc_feature (30)..(261): Final protein product
EFATMAVMAPRTLVLLLSGALALTQTWAGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 39 - DNA, 1440 bp, Homo sapiens
gaattcgccacaatggctgtgatggctccaagaaccctggtcctgctgctgtccggggctctggctctgactcagacctgggccggggaagtgcagctggtcgaatctggaggaggactggtgcagccaggagggtccctgcgcctgtcttgcgccgctagtggcttcacttttaccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctccgcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtggaacccaagtcttgtgataaaacccatacatgccccccttgtcctgcaccagagctgctgggaggaccaagcgtgttcctgtttccacccaagcctaaagatacactgatgattagtaggaccccagaagtcacatgcgtggtcgtggacgtgagccacgaggaccccgaagtcaagtttaactggtacgtggacggcgtcgaggtgcataatgccaagactaaacccagggaggaacagtacaacagtacctatcgcgtcgtgtcagtcctgacagtgctgcatcaggattggctgaacgggaaagagtataagtgcaaagtgagcaataaggctctgcccgcacctatcgagaaaacaatttccaaggcaaaaggacagcctagagaaccacaggtgtacgtgtatcctccatcaagggatgagctgacaaagaaccaggtcagcctgacttgtctggtgaaaggattctatccctctgacattgctgtggagtgggaaagtaatggccagcctgagaacaattacaagaccacaccccctgtgctggactcagatggcagcttcgcgctggtgagcaagctgaccgtcgacaaatcccggtggcagcaggggaatgtgtttagttgttcagtcatgcacgaggcactgcacaaccattacacccagaagtcactgtcactgtcaccagggtgaggatcc

SEQ ID NO: 40 - protein, 477 aa, Homo sapiens
  misc_feature (30)..(477): Final protein product
EFATMAVMAPRTLVLLLSGALALTQTWAGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 41 - DNA, 792 bp, Homo sapiens; identical to SEQ ID NO: 15

SEQ ID NO: 42 - protein, 261 aa, Homo sapiens; identical to SEQ ID NO: 16
  misc_feature (30)..(261): Final protein product

SEQ ID NO: 43 - DNA, 738 bp, Homo sapiens
gaattcgccacaatggctgtgatggcacctagaacactggtcctgctgctgagcggggcactggcactgacacagacttgggccggggatattcagatgacccagtccccaagctccctgagtgcctcagtgggcgaccgagtcaccatcacatgcaaggcttcccaggatgtgtctattggagtcgcatggtaccagcagaagccaggcaaagcacccaagctgctgatctatagcgcctcctaccggtataccggcgtgccctctagattctctggcagtgggtcaggaacagactttactctgaccatctctagtctgcagcctgaggatttcgctacctactattgccagcagtactatatctacccatatacctttggccaggggacaaaagtggagatcaagaggactgtggccgctccctccgtcttcatttttcccccttctgacgaacagctgaaaagtggcacagccagcgtggtctgtctgctgaacaatttctaccctcgcgaagccaaagtgcagtggaaggtcgataacgctctgcagagcggcaacagccaggagtctgtgactgaacaggacagtaaagattcaacctatagcctgtcaagcacactgactctgagcaaggcagactacgagaagcacaaagtgtatgcctgcgaagtcacacatcaggggctgtcctctcctgtgactaagagctttaacagaggagagtgttgaggatcc

SEQ ID NO: 44 - protein, 243 aa, Homo sapiens
  misc_feature (30)..(243): Final protein product
EFATMAVMAPRTLVLLLSGALALTQTWAGDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 792 bp, Homo sapiens; identical to SEQ ID NO: 21

SEQ ID NO: 46 - protein, 261 aa, Homo sapiens; identical to SEQ ID NO: 22
  misc_feature (30)..(261): Final protein product

SEQ ID NO: 47 - DNA, 1440 bp, Homo sapiens
gaattcgccacaatggctgtgatggctccaagaaccctggtcctgctgctgtccggggctctggctctgactcagacctgggccggggaagtgcagctggtcgaatctggaggaggactggtgcagccaggagggtccctgcgcctgtcttgcgccgctagtggcttcacttttaccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctccgcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtggaacccaagtcttgtgataaaacccatacatgccccccttgtcctgcaccagagctgctgggaggaccaagcgtgttcctgtttccacccaagcctaaagatacactgatgattagtaggaccccagaagtcacatgcgtggtcgtggacgtgagccacgaggaccccgaagtcaagtttaactggtacgtggacggcgtcgaggtgcataatgccaagactaaacccagggaggaacagtacaacagtacctatcgcgtcgtgtcagtcctgacagtgctgcatcaggattggctgaacgggaaagagtataagtgcaaagtgagcaataaggctctgcccgcacctatcgagaaaacaatttccaaggcaaaaggacagcctagagaaccacaggtgtacgtgctgcctccatcaagggatgagctgacaaagaaccaggtcagcctgctgtgtctggtgaaaggattctatccctctgacattgctgtggagtgggaaagtaatggccagcctgagaacaattacctgacctggccccctgtgctggactcagatggcagcttctttctgtatagcaagctgaccgtcgacaaatcccggtggcagcaggggaatgtgtttagttgttcagtcatgcacgaggcactgcacaaccattacacccagaagtcactgtcactgtcaccagggtgaggatcc

SEQ ID NO: 48 - protein, 477 aa, Homo sapiens
  misc_feature (30)..(477): Final protein product
EFATMAVMAPRTLVLLLSGALALTQTWAGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 49 - DNA, 738 bp, Homo sapiens; identical to SEQ ID NO: 43

SEQ ID NO: 50 - protein, 243 aa, Homo sapiens; identical to SEQ ID NO: 44
  misc_feature (30)..(243): Final protein product

SEQ ID NO: 51 - DNA, 1440 bp, Homo sapiens; identical to SEQ ID NO: 39

SEQ ID NO: 52 - protein, 477 aa, Homo sapiens; identical to SEQ ID NO: 40
  misc_feature (30)..(477): Final protein product

SEQ ID NO: 53 - DNA, 1440 bp, Homo sapiens; identical to SEQ ID NO: 47

SEQ ID NO: 54 - protein, 477 aa, Homo sapiens; identical to SEQ ID NO: 48
  misc_feature (30)..(477): Final protein product

SEQ ID NO: 55 - DNA, 738 bp, Homo sapiens; identical to SEQ ID NO: 43

SEQ ID NO: 56 - protein, 243 aa, Homo sapiens; identical to SEQ ID NO: 44
  misc_feature (30)..(243): Final protein product

SEQ ID NO: 57 - protein, 450 aa, Artificial Sequence
  Description of Artificial Sequence Synthetic clone 642 Full polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - DNA, 1350 bp, Artificial Sequence
  Description of Artificial Sequence Synthetic clone 642 Full polynucleotide
gaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaa

SEQ ID NO: 59 - protein, 120 aa, Artificial Sequence [sequence omitted]

SEQ ID NO: 60 - DNA, 360 bp, Artificial Sequence
  Description of Artificial Sequence Synthetic clone 642 VH polynucleotide
gaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctcc

SEQ ID NO: 61 - protein, 8 aa, Artificial Sequence
  Description of Artificial Sequence Synthetic clone 642 H1 peptide
GFNIKDTY

SEQ ID NO: 62 - DNA, 24 bp, Artificial Sequence
  Description of Artificial Sequence Synthetic clone 642 H1 oligonucleotide
ggattcaacatcaaggacacctac

SEQ ID NO: 63 - protein, 13 aa, Artificial Sequence
  Description of Artificial Sequence Synthetic clone 642 H3 peptide
SRWGGDGFYAMDY

SEQ ID NO: 64 - DNA, 39 bp, Artificial Sequence
  Description of Artificial Sequence Synthetic clone 642 H3 oligonucleotide
agtcgatggggaggagacggattctacgctatggattat

SEQ ID NO: 65 - protein, 8 aa, Artificial Sequence
  Description of Artificial Sequence Synthetic clone 642 H2 peptide
IYPTNGYT

SEQ ID NO: 66 - DNA, 24 bp, Artificial Sequence
  Description of Artificial Sequence Synthetic clone 642 H2 oligonucleotide
atctatcccactaatggatacacc

SEQ ID NO: 67 - protein, 98 aa, Artificial Sequence
  Description of Artificial Sequence Synthetic clone 642 CH1 polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 68 - DNA, 294 bp, Artificial Sequence
  Description of Artificial Sequence Synthetic clone 642 CH1 polynucleotide
gcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtg

SEQ ID NO: 69 - protein, 110 aa, Artificial Sequence
  Description of Artificial Sequence Synthetic clone 642 CH2 polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 70 - DNA, 330 bp, Artificial Sequence
  Description of Artificial Sequence Synthetic clone 642 CH2 polynucleotide
gctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaa

SEQ ID NO: 71 - protein, 106 aa, Artificial Sequence [sequence omitted]

SEQ ID NO: 72 - DNA, 318 bp, Artificial Sequence
  Description of Artificial Sequence Synthetic clone 642 CH3 polynucleotide
ggccagccaagggagccccaggtgtacacactgccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggc

SEQ ID NO: 73 - protein, 232 aa, Artificial Sequence
  Description of Artificial Sequence Synthetic clone 716 Full polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLICLVKGFYPSDIAVEWESNGQPENRYMTWPPVLDSDGSFFLYSKLTVDKSRWQQGNVFSCSVMHEALHNHYTQKSLSLSPGK

SEQ ID NO: 74 - DNA, 696 bp, Artificial Sequence
  Description of Artificial Sequence Synthetic clone 716 Full polynucleotide
gagcccaagagcagcgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacactgccacccagcagagacgaactgaccaagaaccaggtgtccctgatctgtctggtgaaaggcttctatcctagtgatattgctgtggagtgggaatcaaatggacagccagagaacagatacatgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaa

SEQ ID NO: 75 - protein, 110 aa, Artificial Sequence; identical to SEQ ID NO: 69
  Description of Artificial Sequence Synthetic clone 716 CH2 polypeptide

SEQ ID NO: 76 - DNA, 330 bp, Artificial Sequence; identical to SEQ ID NO: 70
  Description of Artificial Sequence Synthetic clone 716 CH2 polynucleotide

SEQ ID NO: 77 - protein, 106 aa, Artificial Sequence
  Description of Artificial Sequence Synthetic clone 716 CH3 polypeptide
GQPREPQVYTLPPSRDELTKNQVSLICLVKGFYPSDIAVEWESNGQPENRYMTWPPVLDSDGSFFLYSKLTVDKSRWQQGNVFSCSVMHEALHNHYTQKSLSLSPG

SEQ ID NO: 78 - DNA, 318 bp, Artificial Sequence
  Description of Artificial Sequence Synthetic clone 716 CH3 polynucleotide
ggccagccaagggagccccaggtgtacacactgccacccagcagagacgaactgaccaagaaccaggtgtccctgatctgtctggtgaaaggcttctatcctagtgatattgctgtggagtgggaatcaaatggacagccagagaacagatacatgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggc

SEQ ID NO: 79 - protein, 232 aa, Artificial Sequence
  Description of Artificial Sequence Synthetic clone 1039 Full polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 80 - DNA, 696 bp, Artificial Sequence
  Description of Artificial Sequence Synthetic clone 1039 Full polynucleotide
gaacctaagagctccgacaaaacccacacatgccccccttgtccagctccagaactgctgggaggaccatccgtgttcctgtttccacccaagcccaaagatacactgatgatctctcgaactcccgaggtcacctgcgtggtcgtggacgtcagtcacgaggaccccgaagtcaagttcaactggtacgtggacggcgtcgaagtgcataatgcaaagactaaaccacgggaggaacagtacaactcaacatatagagtcgtgagcgtcctgactgtgctgcatcaggattggctgaacggcaaggagtataagtgcaaagtgagcaataaggccctgcctgctccaatcgagaaaaccattagcaaggcaaaagggcagcccagggaacctcaggtgtacaccctgcctccaagccgcgacgagctgacaaagaaccaggtctccctgctgtgtctggtgaaaggattctatcctagtgatattgccgtggagtgggaatcaaatggccagccagagaacaattacatgacttggccccctgtgctggactctgatgggagtttctttctgtattccaagctgaccgtggacaaatctagatggcagcagggaaacgtcttttcttgtagtgtgatgcacgaagccctgcacaatcattacacacagaagtcactgagcctgtcccctggcaaa

SEQ ID NO: 81 - protein, 110 aa, Artificial Sequence; identical to SEQ ID NO: 69
  Description of Artificial Sequence Synthetic clone 1039 CH2 polypeptide

SEQ ID NO: 82 - DNA, 330 bp, Artificial Sequence
  Description of Artificial Sequence Synthetic clone 1039 CH2 polynucleotide
gctccagaactgctgggaggaccatccgtgttcctgtttccacccaagcccaaagatacactgatgatctctcgaactcccgaggtcacctgcgtggtcgtggacgtcagtcacgaggaccccgaagtcaagttcaactggtacgtggacggcgtcgaagtgcataatgcaaagactaaaccacgggaggaacagtacaactcaacatatagagtcgtgagcgtcctgactgtgctgcatcaggattggctgaacggcaaggagtataagtgcaaagtgagcaataaggccctgcctgctccaatcgagaaaaccattagcaaggcaaaa

SEQ ID NO: 83 - protein, 106 aa, Artificial Sequence
  Description of Artificial Sequence Synthetic clone 1039 CH3 polypeptide
GQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPG

SEQ ID NO: 84 - DNA, 318 bp, Artificial Sequence
  Description of Artificial Sequence Synthetic clone 1039 CH3 polynucleotide
gggcagcccagggaacctcaggtgtacaccctgcctccaagccgcgacgagctgacaaagaaccaggtctccctgctgtgtctggtgaaaggattctatcctagtgatattgccgtggagtgggaatcaaatggccagccagagaacaattacatgacttggccccctgtgctggactctgatgggagtttctttctgtattccaagctgaccgtggacaaatctagatggcagcagggaaacgtcttttcttgtagtgtgatgcacgaagccctgcacaatcattacacacagaagtcactgagcctgtcccctggc

SEQ ID NO: 85 - protein, 214 aa, Artificial Sequence
  Description of Artificial Sequence Synthetic clone 1811 Full polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - DNA, 642 bp, Artificial Sequence
  Description of Artificial Sequence Synthetic clone 1811 Full polynucleotide
gatattcagatgacccagtccccaagctccctgagtgcctcagtgggcgaccgagtcaccatcacatgcaaggcttcccaggatgtgtctattggagtcgcatggtaccagcagaagccaggcaaagcacccaagctgctgatctatagcgcctcctaccggtataccggcgtgccctctagattctctggcagtgggtcaggaacagactttactctgaccatctctagtctgcagcctgaggatttcgctacctactattgccagcagtactatatctacccatatacctttggccaggggacaaaagtggagatcaagaggactgtggccgctccctccgtcttcatttttcccccttctgacgaacagctgaaaagtggcacagccagcgtggtctgtctgctgaacaatttctaccctcgcgaagccaaagtgcagtggaaggtcgataacgctctgcagagcggcaacagccaggagtctgtgactgaacaggacagtaaagattcaacctatagcctgtcaagcacactgactctgagcaaggcagactacgagaagcacaaagtgtatgcctgcgaagtcacacatcaggggctgtcctctcctgtgactaagagctttaacagaggagagtgt

SEQ ID NO: 87 - protein, 107 aa, Artificial Sequence [sequence omitted]

SEQ ID NO: 88 - DNA, 321 bp, Artificial Sequence
  Description of Artificial Sequence Synthetic clone 1811 VL polynucleotide
gatattcagatgacccagtccccaagctccctgagtgcctcagtgggcgaccgagtcaccatcacatgcaaggcttcccaggatgtgtctattggagtcgcatggtaccagcagaagccaggcaaagcacccaagctgctgatctatagcgcctcctaccggtataccggcgtgccctctagattctctggcagtgggtcaggaacagactttactctgaccatctctagtctgcagcctgaggatttcgctacctactattgccagcagtactatatctacccatatacctttggccaggggacaaaagtggagatcaag

SEQ ID NO: 89 - protein, 6 aa, Artificial Sequence
  Description of Artificial Sequence Synthetic clone 1811 L1 peptide
QDVSIG

SEQ ID NO: 90 - DNA, 18 bp, Artificial Sequence
  Description of Artificial Sequence Synthetic clone 1811 L1 oligonucleotide
caggatgtgtctattgga

SEQ ID NO: 91 - protein, 9 aa, Artificial Sequence
  Description of Artificial Sequence Synthetic clone 1811 L3 peptide
QQYYIYPYT

SEQ ID NO: 92 - DNA, 27 bp, Artificial Sequence
  Description of Artificial Sequence Synthetic clone 1811 L3 oligonucleotide
cagcagtactatatctacccatatacc

SEQ ID NO: 93 - protein, 3 aa, Artificial Sequence
  Description of Artificial Sequence Synthetic clone 1811 L2 peptide
SAS

SEQ ID NO: 94 - DNA, 9 bp, Artificial Sequence
  Description of Artificial Sequence Synthetic clone 1811 L2 oligonucleotide
agcgcctcc

SEQ ID NO: 95 - protein, 107 aa, Artificial Sequence [sequence omitted]

SEQ ID NO: 96 - DNA, 321 bp, Artificial Sequence
  Description of Artificial Sequence Synthetic clone 1811 CL polynucleotide
aggactgtggccgctccctccgtcttcatttttcccccttctgacgaacagctgaaaagtggcacagccagcgtggtctgtctgctgaacaatttctaccctcgcgaagccaaagtgcagtggaaggtcgataacgctctgcagagcggcaacagccaggagtctgtgactgaacaggacagtaaagattcaacctatagcctgtcaagcacactgactctgagcaaggcagactacgagaagcacaaagtgtatgcctgcgaagtcacacatcaggggctgtcctctcctgtgactaagagctttaacagaggagagtgt

SEQ ID NO: 97 - protein, 489 aa, Artificial Sequence
  Description of Artificial Sequence Synthetic clone 1070 Full polypeptide
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSSGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 98 - DNA, 1467 bp, Artificial Sequence
  Description of Artificial Sequence Synthetic clone 1070 Full polynucleotide
caggtccagctggtgcagagcggggcagaggtcaagaaacccggagaaagtctgaagatctcatgcaaagggagtggatactcattcaccagctattggattgcctgggtgaggcagatgcctggcaaggggctggaatacatgggcctgatctatccaggggacagcgatacaaaatactccccctctttccagggccaggtcacaatttccgtggacaagagtgtctcaactgcctatctgcagtggagctccctgaaacctagcgattccgcagtgtacttttgtgccaggcacgacgtcgggtattgcacagatcgcacttgtgctaagtggccagagtggctgggagtgtggggacagggaaccctggtcacagtgtctagtggaggaggaggctcaagcggaggaggctctggaggaggagggtctcagagtgtgctgactcagccaccttcagtcagcgcagctcctggacagaaggtgaccatctcctgctctggcagctctagtaacattggcaacaattacgtgagctggtatcagcagctgcctggcaccgccccaaagctgctgatctacgaccacacaaatcggcccgctggggtgcctgatagattcagtgggtcaaaaagcggaacctccgcttctctggcaattagcggctttcgctccgaggacgaagctgattactattgtgcatcttgggactacacactgagtggctgggtgttcggaggcgggactaagctgaccgtgctgggggcagccgaaccaaagtcaagcgataaaactcatacctgcccaccatgtcctgcaccagagctgctgggaggaccttccgtgttcctgtttcctccaaagccaaaagacaccctgatgatcagccgaacaccagaagtgacttgcgtggtcgtggacgtctcccacgaggaccccgaagtgaagtttaactggtacgtggatggcgtcgaggtgcataatgccaagaccaaaccccgagaggaacagtacaactcaacttatcgggtcgtgagcgtcctgaccgtgctgcaccaggactggctgaacgggaaagagtataagtgcaaagtgtctaataaggccctgcccgctcctatcgagaaaacaattagcaaggcaaaaggccagccaagagaaccccaggtgtacacttatcccccttctagggacgagctgaccaagaaccaggtgagcctgacatgtctggtcaaaggattttaccccagtgatattgctgtggagtgggaatccaatggccagcctgaaaacaattataagaccacaccacccgtgctggactccgatggatctttcgctctggtgtccaagctgactgtcgataaatctcggtggcagcagggcaacgtgtttagttgttcagtcatgcatgaggcactgcacaatcattacacacagaagagcctgtccctgtctcccggcaaa

SEQ ID NO: 99 - protein, 129 aa, Artificial Sequence
  Description of Artificial Sequence Synthetic clone 1070 VH polypeptide
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSS

SEQ ID NO: 100 - DNA, 387 bp, Artificial Sequence
  Description of Artificial Sequence Synthetic clone 1070 VH polynucleotide
caggtccagctggtgcagagcggggcagaggtcaagaaacccggagaaagtctgaagatctcatgcaaagggagtggatactcattcaccagctattggattgcctgggtgaggcagatgcctggcaaggggctggaatacatgggcctgatctatccaggggacagcgatacaaaatactccccctctttccagggccaggtcacaatttccgtggacaagagtgtctcaactgcctatctgcagtggagctccctgaaacctagcgattccgcagtgtacttttgtgccaggcacgacgtcgggtattgcacagatcgcacttgtgctaagtggccagagtggctgggagtgtggggacagggaaccctggtcacagtgtctagt

SEQ ID NO: 101 - protein, 8 aa, Artificial Sequence
  Description of Artificial Sequence Synthetic clone 1070 H1 peptide
GYSFTSYW

SEQ ID NO: 102 - DNA, 24 bp, Artificial Sequence
  Description of Artificial Sequence Synthetic clone 1070 H1 oligonucleotide
ggatactcattcaccagctattgg

SEQ ID NO: 103 - protein, 22 aa, Artificial Sequence
  Description of Artificial Sequence Synthetic clone 1070 H3 peptide
ARHDVGYCTDRTCAKWPEWLGV

SEQ ID NO: 104 - DNA, 66 bp, Artificial Sequence
  Description of Artificial Sequence Synthetic clone 1070 H3 oligonucleotide
gccaggcacgacgtcgggtattgcacagatcgcacttgtgctaagtggccagagtggctgggagtg

SEQ ID NO: 105 - protein, 8 aa, Artificial Sequence
  Description of Artificial Sequence Synthetic clone 1070 H2 peptide
IYPGDSDT

SEQ ID NO: 106 - DNA, 24 bp, Artificial Sequence
  Description of Artificial Sequence Synthetic clone 1070 H2 oligonucleotide
atctatccaggggacagcgataca

SEQ ID NO: 107 - protein, 110 aa, Artificial Sequence
  Description of Artificial Sequence Synthetic clone 1070 VL polypeptide
QSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVL

SEQ ID NO: 108 - DNA, 330 bp, Artificial Sequence
  Description of Artificial Sequence Synthetic clone 1070 VL polynucleotide
cagagtgtgctgactcagccaccttcagtcagcgcagctcctggacagaaggtgaccatctcctgctctggcagctctagtaacattggcaacaattacgtgagctggtatcagcagctgcctggcaccgccccaaagctgctgatctacgaccacacaaatcggcccgctggggtgcctgatagattcagtgggtcaaaaagcggaacctccgcttctctggcaattagcggctttcgctccgaggacgaagctgattactattgtgcatcttgggactacacactgagtggctgggtgttcggaggcgggactaagctgaccgtgctg

SEQ ID NO: 109 - protein, 8 aa, Artificial Sequence
  Description of Artificial Sequence Synthetic clone 1070 L1 peptide
SSNIGNNY

SEQ ID NO: 110 - DNA, 24 bp, Artificial Sequence
  Description of Artificial Sequence Synthetic clone 1070 L1 oligonucleotide
tctagtaacattggcaacaattac

SEQ ID NO: 111 - protein, 11 aa, Artificial Sequence
  Description of Artificial Sequence Synthetic clone 1070 L3 peptide
ASWDYTLSGWV

SEQ ID NO: 112 - DNA, 33 bp, Artificial Sequence
  Description of Artificial Sequence Synthetic clone 1070 L3 oligonucleotide
gcatcttgggactacacactgagtggctgggtg

SEQ ID NO: 113 - protein, 3 aa, Artificial Sequence
  Description of Artificial Sequence Synthetic clone 1070 L2 peptide
DHT

SEQ ID NO: 114 - DNA, 9 bp, Artificial Sequence
  Description of Artificial Sequence Synthetic clone 1070 L2 oligonucleotide
gaccacaca

SEQ ID NO: 115 - protein, 110 aa, Artificial Sequence; identical to SEQ ID NO: 69
  Description of Artificial Sequence Synthetic clone 1070 CH2 polypeptide

SEQ ID NO: 116 - DNA, 330 bp, Artificial Sequence
  Description of Artificial Sequence Synthetic clone 1070 CH2 polynucleotide
gcaccagagctgctgggaggaccttccgtgttcctgtttcctccaaagccaaaagacaccctgatgatcagccgaacaccagaagtgacttgcgtggtcgtggacgtctcccacgaggaccccgaagtgaagtttaactggtacgtggatggcgtcgaggtgcataatgccaagaccaaaccccgagaggaacagtacaactcaacttatcgggtcgtgagcgtcctgaccgtgctgcaccaggactggctgaacgggaaagagtataagtgcaaagtgtctaataaggccctgcccgctcctatcgagaaaacaattagcaaggcaaaa

SEQ ID NO: 117 - protein, 106 aa, Artificial Sequence
  Description of Artificial Sequence Synthetic clone 1070 CH3 polypeptide
GQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 118 - DNA, 318 bp, Artificial Sequence
  Description of Artificial Sequence Synthetic clone 1070 CH3 polynucleotide
ggccagccaagagaaccccaggtgtacacttatcccccttctagggacgagctgaccaagaaccaggtgagcctgacatgtctggtcaaaggattttaccccagtgatattgctgtggagtgggaatccaatggccagcctgaaaacaattataagaccacaccacccgtgctggactccgatggatctttcgctctggtgtccaagctgactgtcgataaatctcggtggcagcagggcaacgtgtttagttgttcagtcatgcatgaggcactgcacaatcattacacacagaagagcctgtccctgtctcccggc

SEQ ID NO: 119 - protein, 448 aa, Artificial Sequence
  Description of Artificial Sequence Synthetic clone 3376 Full polypeptide
EVQLVESGGGLVQPGGSLRLSCAASGFTFADYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 120 - DNA, 1344 bp, Artificial Sequence
  Description of Artificial Sequence Synthetic clone 3376 Full polynucleotide
gaagtgcagctggtcgaatctggaggaggactggtgcagccaggagggtccctgcgcctgtcttgcgccgctagtggcttcacttttgccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctccgcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtggaacccaagtcttgtgataaaacccatacatgccccccttgtcctgcaccagagctgctgggaggaccaagcgtgttcctgtttccacccaagcctaaagatacactgatgattagtaggaccccagaagtcacatgcgtggtcgtggacgtgagccacgaggaccccgaagtcaagtttaactggtacgtggacggcgtcgaggtgcataatgccaagactaaacccagggaggaacagtacaacagtacctatcgcgtcgtgtcagtcctgacagtgctgcatcaggattggctgaacgggaaagagtataagtgcaaagtgagcaataaggctctgcccgcacctatcgagaaaacaatttccaaggcaaaaggacagcctagagaaccacaggtgtacgtgtatcctccatcaagggatgagctgacaaagaaccaggtcagcctgacttgtctggtgaaaggattctatccctctgacattgctgtggagtgggaaagtaatggccagcctgagaacaattacaagaccacaccccctgtgctggactcagatggcagcttcgcgctggtgagcaagctgaccgtcgacaaatcccggtggcagcaggggaatgtgtttagttgttcagtcatgcacgaggcactgcacaaccattacacccagaagtcactgtcactgtcaccaggg

SEQ ID NO: 121 - protein, 119 aa, Artificial Sequence
  Description of Artificial Sequence Synthetic clone 3376 VH polypeptide
EVQLVESGGGLVQPGGSLRLSCAASGFTFADYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 122 - DNA, 357 bp, Artificial Sequence
  Description of Artificial Sequence Synthetic clone 3376 VH polynucleotide
gaagtgcagctggtcgaatctggaggaggactggtgcagccaggagggtccctgcgcctgtcttgcgccgctagtggcttcacttttgccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctcc

SEQ ID NO: 123 - protein, 8 aa, Artificial Sequence
  Description of Artificial Sequence Synthetic clone 3376 H1 peptide
GFTFADYT

SEQ ID NO: 124 - DNA, 24 bp, Artificial Sequence
  Description of Artificial Sequence Synthetic clone 3376 H1 oligonucleotide
ggcttcacttttgccgactacacc

SEQ ID NO: 125 - protein, 12 aa, Artificial Sequence
  Description of Artificial Sequence Synthetic clone 3376 H3 peptide
ARNLGPSFYFDY

SEQ ID NO: 126 - DNA, 36 bp, Artificial Sequence
  Description of Artificial Sequence Synthetic clone 3376 H3 oligonucleotide
gcccggaatctggggccctccttctactttgactat

SEQ ID NO: 127 - protein, 8 aa, Artificial Sequence
  Description of Artificial Sequence Synthetic clone 3376 H2 peptide
VNPNSGGS

SEQ ID NO: 128 - DNA, 24 bp, Artificial Sequence
  Description of Artificial Sequence Synthetic clone 3376 H2 oligonucleotide
gtgaacccaaatagcggaggctcc

SEQ ID NO: 129 - protein, 98 aa, Artificial Sequence; identical to SEQ ID NO: 67
  Description of Artificial Sequence Synthetic clone 3376 CH1 polypeptide

SEQ ID NO: 130 - DNA, 294 bp, Artificial Sequence
  Description of Artificial Sequence Synthetic clone 3376 CH1 polynucleotide
gcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtg

SEQ ID NO: 131 - protein, 110 aa, Artificial Sequence; identical to SEQ ID NO: 69
  Description of Artificial Sequence Synthetic clone 3376 CH2 polypeptide

SEQ ID NO: 132 - DNA, 330 bp, Artificial Sequence
  Description of Artificial Sequence Synthetic clone 3376 CH2 polynucleotide
gcaccagagctgctgggaggaccaagcgtgttcctgtttccacccaagcctaaagatacactgatgattagtaggaccccagaagtcacatgcgtggtcgtggacgtgagccacgaggaccccgaagtcaagtttaactggtacgtggacggcgtcgaggtgcataatgccaagactaaacccagggaggaacagtacaacagtacctatcgcgtcgtgtcagtcctgacagtgctgcatcaggattggctgaacgggaaagagtataagtgcaaagtgagcaataaggctctgcccgcacctatcgagaaaacaatttccaaggcaaaa

SEQ ID NO: 133 - protein, 106 aa, Artificial Sequence
  Description of Artificial Sequence Synthetic clone 3376 CH3 polypeptide
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 134 - DNA, 318 bp, Artificial Sequence
  Description of Artificial Sequence Synthetic clone 3376 CH3 polynucleotide
ggacagcctagagaaccacaggtgtacgtgtatcctccatcaagggatgagctgacaaagaaccaggtcagcctgacttgtctggtgaaaggattctatccctctgacattgctgtggagtgggaaagtaatggccagcctgagaacaattacaagaccacaccccctgtgctggactcagatggcagcttcgcgctggtgagcaagctgaccgtcgacaaatcccggtggcagcaggggaatgtgtttagttgttcagtcatgcacgaggcactgcacaaccattacacccagaagtcactgtcactgtcaccaggg

SEQ ID NO: 135 - protein, 448 aa, Artificial Sequence
  Description of Artificial Sequence Synthetic clone 3379 Full polypeptide
EVQLVESGGGLVQPGGSLRLSCAASGFTFADYTMDWVRQAPGKGLEWVADVNPNSGASIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 136 - DNA, 1344 bp, Artificial Sequence
  Description of Artificial Sequence Synthetic clone 3379 Full polynucleotide
gaagtgcagctggtcgaatctggaggaggactggtgcagccaggagggtccctgcgcctgtcttgcgccgctagtggcttcacttttgccgactacaccatggattgggtgcgacaggcacctggaaagggcctggagtgggtcgccgatgtgaacccaaatagcggagcctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctccgcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtggaacccaagtcttgtgataaaacccatacatgccccccttgtcctgcaccagagctgctgggaggaccaagcgtgttcctgtttccacccaagcctaaagatacactgatgattagtaggaccccagaagtcacatgcgtggtcgtggacgtgagccacgaggaccccgaagtcaagtttaactggtacgtggacggcgtcgaggtgcataatgccaagactaaacccagggaggaacagtacaacagtacctatcgcgtcgtgtcagtcctgacagtgctgcatcaggattggctgaacgggaaagagtataagtgcaaagtgagcaataaggctctgcccgcacctatcgagaaaacaatttccaaggcaaaaggacagcctagagaaccacaggtgtacgtgtatcctccatcaagggatgagctgacaaagaaccaggtcagcctgacttgtctggtgaaaggattctatccctctgacattgctgtggagtgggaaagtaatggccagcctgagaacaattacaagaccacaccccctgtgctggactcagatggcagcttcgcgctggtgagcaagctgaccgtcgacaaatcccggtggcagcaggggaatgtgtttagttgttcagtcatgcacgaggcactgcacaaccattacacccagaagtcactgtcactgtcaccaggg

SEQ ID NO: 137 - protein, 119 aa, Artificial Sequence
  Description of Artificial Sequence Synthetic clone 3379 VH polypeptide
EVQLVESGGGLVQPGGSLRLSCAASGFTFADYTMDWVRQAPGKGLEWVADVNPNSGASIYNQRFKGRFTLSVDRSKNTLYLQMNSLRAEDTAVYYCARNLGPSFYFDYWGQGTLVTVSS

SEQ ID NO: 138 - DNA, 357 bp, Artificial Sequence
  Description of Artificial Sequence Synthetic clone 3379 VH polynucleotide
gaagtgcagctggtcgaatctggaggaggactggtgcagccaggagggtccctgcgcctgtcttgcgccgctagtggcttcacttttgccgactacaccatggattgggtgcgacaggcacctggaaagggcctggagtgggtcgccgatgtgaacccaaatagcggagcctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctcc

SEQ ID NO: 139 - protein, 8 aa, Artificial Sequence
  Description of Artificial Sequence Synthetic clone 3379 H1 peptide
GFTFADYT

SEQ ID NO: 140 - DNA, 24 bp, Artificial Sequence
  Description of Artificial Sequence Synthetic clone 3379 H1 oligonucleotide
ggcttcacttttgccgactacacc

SEQ ID NO: 141 - protein, 12 aa, Artificial Sequence
  Description of Artificial Sequence Synthetic clone 3379 H3 peptide
ARNLGPSFYFDY

SEQ ID NO: 142 - DNA, 36 bp, Artificial Sequence; identical to SEQ ID NO: 126
  Description of Artificial Sequence Synthetic clone 3379 H3 oligonucleotide

SEQ ID NO: 143 - protein, 8 aa, Artificial Sequence
  Description of Artificial Sequence Synthetic clone 3379 H2 peptide
VNPNSGAS

SEQ ID NO: 144 - DNA, 24 bp, Artificial Sequence
  Description of Artificial Sequence Synthetic clone 3379 H2 oligonucleotide
gtgaacccaaatagcggagcctcc

SEQ ID NO: 145 - protein, 98 aa, Artificial Sequence; identical to SEQ ID NO: 67
  Description of Artificial Sequence Synthetic clone 3379 CH1 polypeptide

SEQ ID NO: 146 - DNA, 294 bp, Artificial Sequence; identical to SEQ ID NO: 130
  Description of Artificial Sequence Synthetic clone 3379 CH1 polynucleotide

SEQ ID NO: 147 - protein, 110 aa, Artificial Sequence; identical to SEQ ID NO: 69
  Description of Artificial Sequence Synthetic clone 3379 CH2 polypeptide

SEQ ID NO: 148 - DNA, 330 bp, Artificial Sequence; identical to SEQ ID NO: 132
  Description of Artificial Sequence Synthetic clone 3379 CH2 polynucleotide

SEQ ID NO: 149 - protein, 106 aa, Artificial Sequence; identical to SEQ ID NO: 133
  Description of Artificial Sequence Synthetic clone 3379 CH3 polypeptide

SEQ ID NO: 150 - DNA, 318 bp, Artificial Sequence; identical to SEQ ID NO: 134
  Description of Artificial Sequence Synthetic clone 3379 CH3 polynucleotide

SEQ ID NO: 151 - protein, 214 aa, Artificial Sequence
  Description of Artificial Sequence Synthetic clone 3382 Full polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - DNA, 642 bp, Artificial Sequence
  Description of Artificial Sequence Synthetic clone 3382 Full polynucleotide
gatattcagatgacccagtccccaagctccctgagtgcctcagtgggcgaccgagtcaccatcacatgcaaggcttcccaggatgtgtctattggagtcgcatggtaccagcagaagccaggcaaagcacccaagctgctgatctatagcgcctcctaccggtataccggcgtgccctctagattctctggcagtgggtcaggaacagactttactctgaccatctctagtctgcagcctgaggatttcgctacctactattgccagcagtactatatctacccagccacctttggccaggggacaaaagtggagatcaagaggactgtggccgctccctccgtcttcatttttcccccttctgacgaacagctgaaaagtggcacagccagcgtggtctgtctgctgaacaatttctaccctcgcgaagccaaagtgcagtggaaggtcgataacgctctgcagagcggcaacagccaggagtctgtgactgaacaggacagtaaagattcaacctatagcctgtcaagcacactgactctgagcaaggcagactacgagaagcacaaagtgtatgcctgcgaagtcacacatcaggggctgtcctctcctgtgactaagagctttaacagaggagagtgt

SEQ ID NO: 153 - protein, 107 aa, Artificial Sequence
  Description of Artificial Sequence Synthetic clone 3382 VL polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPATFGQGTKVEIK

SEQ ID NO: 154 - DNA, 321 bp, Artificial Sequence
  Description of Artificial Sequence Synthetic clone 3382 VL polynucleotide
gatattcagatgacccagtccccaagctccctgagtgcctcagtgggcgaccgagtcaccatcacatgcaaggcttcccaggatgtgtctattggagtcgcatggtaccagcagaagccaggcaaagcacccaagctgctgatctatagcgcctcctaccggtataccggcgtgccctctagattctctggcagtgggtcaggaacagactttactctgaccatctctagtctgcagcctgaggatttcgctacctactattgccagcagtactatatctacccagccacctttggccaggggacaaaagtggagatcaag

SEQ ID NO: 155 - protein, 6 aa, Artificial Sequence
  Description of Artificial Sequence Synthetic clone 3382 L1 peptide
QDVSIG

SEQ ID NO: 156 - DNA, 18 bp, Artificial Sequence
  Description of Artificial Sequence Synthetic clone 3382 L1 oligonucleotide
caggatgtgtctattgga

SEQ ID NO: 157 - protein, 9 aa, Artificial Sequence
  Description of Artificial Sequence Synthetic clone 3382 L3 peptide
QQYYIYPAT

SEQ ID NO: 158 - DNA, 27 bp, Artificial Sequence
  Description of Artificial Sequence Synthetic clone 3382 L3 oligonucleotide
cagcagtactatatctacccagccacc

SEQ ID NO: 159 - protein, 3 aa, Artificial Sequence
  Description of Artificial Sequence Synthetic clone 3382 L2 peptide
SAS

SEQ ID NO: 160 - DNA, 9 bp, Artificial Sequence
  Description of Artificial Sequence Synthetic clone 3382 L2 oligonucleotide
agcgcctcc

SEQ ID NO: 161 - protein, 107 aa, Artificial Sequence [sequence omitted]

SEQ ID NO: 162 - DNA, 321 bp, Artificial Sequence; identical to SEQ ID NO: 96
  Description of Artificial Sequence Synthetic clone 3382 CL polynucleotide

SEQ ID NO: 163 - protein, 214 aa, Artificial Sequence
  Description of Artificial Sequence Synthetic clone 3383 Full polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - DNA, 642 bp, Artificial Sequence
  Description of Artificial Sequence Synthetic clone 3383 Full polynucleotide
gatattcagatgacccagtccccaagctccctgagtgcctcagtgggcgaccgagtcaccatcacatgcaaggcttcccaggatgtgtctattggagtcgcatggtaccagcagaagccaggcaaagcacccaagctgctgatctatagcgcctcctaccggtataccggcgtgccctctagattctctggcagtgggtcaggaacagactttactctgaccatctctagtctgcagcctgaggatttcgctacctactattgccagcagtactatatctacccattcacctttggccaggggacaaaagtggagatcaagaggactgtggccgctccctccgtcttcatttttcccccttctgacgaacagctgaaaagtggcacagccagcgtggtctgtctgctgaacaatttctaccctcgcgaagccaaagtgcagtggaaggtcgataacgctctgcagagcggcaacagccaggagtctgtgactgaacaggacagtaaagattcaacctatagcctgtcaagcacactgactctgagcaaggcagactacgagaagcacaaagtgtatgcctgcgaagtcacacatcaggggctgtcctctcctgtgactaagagctttaacagaggagagtgt

SEQ ID NO: 165 - protein, 107 aa, Artificial Sequence
  Description of Artificial Sequence Synthetic clone 3383 VL polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPFTFGQGTKVEIK

SEQ ID NO: 166 - DNA, 321 bp, Artificial Sequence
  Description of Artificial Sequence Synthetic clone 3383 VL polynucleotide
gatattcagatgacccagtccccaagctccctgagtgcctcagtgggcgaccgagtcaccatcacatgcaaggcttcccaggatgtgtctattggagtcgcatggtaccagcagaagccaggcaaagcacccaagctgctgatctatagcgcctcctaccggtataccggcgtgccctctagattctctggcagtgggtcaggaacagactttactctgaccatctctagtctgcagcctgaggatttcgctacctactattgccagcagtactatatctacccattcacctttggccaggggacaaaagtggagatcaag

SEQ ID NO: 167 - protein, 6 aa, Artificial Sequence
  Description of Artificial Sequence Synthetic clone 3383 L1 peptide
QDVSIG

SEQ ID NO: 168 - DNA, 18 bp, Artificial Sequence
  Description of Artificial Sequence Synthetic clone 3383 L1 oligonucleotide
caggatgtgtctattgga

SEQ ID NO: 169 - protein, 9 aa, Artificial Sequence
  Description of Artificial Sequence Synthetic clone 3383 L3 peptide
QQYYIYPFT

SEQ ID NO: 170 - DNA, 27 bp, Artificial Sequence
  Description of Artificial Sequence Synthetic clone 3383 L3 oligonucleotide
cagcagtactatatctacccattcacc

SEQ ID NO: 171 - protein, 3 aa, Artificial Sequence
  Description of Artificial Sequence Synthetic clone 3383 L2 peptide
SAS

SEQ ID NO: 172 - DNA, 9 bp, Artificial Sequence
  Description of Artificial Sequence Synthetic clone 3383 L2 oligonucleotide
agcgcctcc

SEQ ID NO: 173 - protein, 107 aa, Artificial Sequence [sequence omitted]

SEQ ID NO: 174 - DNA, 321 bp, Artificial Sequence; identical to SEQ ID NO: 96
  Description of Artificial Sequence Synthetic clone 3383 CL polynucleotide

SEQ ID NO: 175 - protein, 450 aa, Artificial Sequence
  Description of Artificial Sequence Synthetic clone 4553 Full polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 176 - DNA, 1350 bp, Artificial Sequence
  Description of Artificial Sequence Synthetic clone 4553 Full polynucleotide
gaagtccagctggtcgaaagcggaggaggactggtgcagccaggagggtctctgcgactgagttgcgccgcttcaggcttcaacatcaaggacacctacattcactgggtgcgccaggctcctggaaaaggcctggagtgggtggcacgaatctatccaactaatggatacacccggtatgcagacagcgtgaagggccggttcaccattagcgcagatacatccaaaaacactgcctacctgcagatgaacagcctgcgagccgaagatactgctgtgtactattgcagtcggtggggaggcgacggcttctacgctatggattattgggggcagggaaccctggtcacagtgagctccgcatctacaaaggggcctagtgtgtttccactggccccctctagtaaatccacctctgggggaacagcagccctgggatgtctggtgaaggactatttcccagagcccgtcactgtgagttggaactcaggcgccctgacatccggggtccatacttttcctgctgtgctgcagtcaagcggcctgtactctctgtcctctgtggtcaccgtgccaagttcaagcctggggactcagacctatatctgcaacgtgaatcacaagccaagcaatacaaaagtcgacaagaaagtggaacccaagagctgtgataaaacacatacttgccccccttgtcctgcaccagagctgctgggaggaccatccgtgttcctgtttccacccaagcctaaagacaccctgatgatttccaggactccagaagtcacctgcgtggtcgtggacgtgtctcacgaggaccccgaagtcaagttcaactggtacgtggatggcgtcgaggtgcataatgccaagacaaaacccagggaggaacagtacaactcaacttatcgcgtcgtgagcgtcctgaccgtgctgcaccaggactggctgaacggcaaggagtataagtgcaaagtgagcaataaggctctgcccgcacctatcgagaaaaccattagcaaggccaaagggcagcctagagaaccacaggtctacgtgtatcctccaagcagggacgagctgaccaagaaccaggtctccctgacatgtctggtgaaagggttttaccccagtgatatcgctgtggagtgggaatcaaatggacagcctgaaaacaattataagaccacaccccctgtgctggacagcgatggcagcttcgctctggtctccaagctgactgtggataaatctcggtggcagcagggcaacgtctttagttgttcagtgatgcatgaggcactgcacaatcattacacccagaagagcctgtccctgtctcccggcaaa

SEQ ID NO: 177 - protein, 120 aa, Artificial Sequence [sequence omitted]

SEQ ID NO: 178 - DNA, 360 bp, Artificial Sequence
  Description of Artificial Sequence Synthetic clone 4553 VH polynucleotide
gaagtccagctggtcgaaagcggaggaggactggtgcagccaggagggtctctgcgactgagttgcgccgcttcaggcttcaacatcaaggacacctacattcactgggtgcgccaggctcctggaaaaggcctggagtgggtggcacgaatctatccaactaatggatacacccggtatgcagacagcgtgaagggccggttcaccattagcgcagatacatccaaaaacactgcctacctgcagatgaacagcctgcgagccgaagatactgctgtgtactattgcagtcggtggggaggcgacggcttctacgctatggattattgggggcagggaaccctggtcacagtgagctcc

SEQ ID NO: 179 - protein, 8 aa, Artificial Sequence
  Description of Artificial Sequence Synthetic clone 4553 H1 peptide
GFNIKDTY

SEQ ID NO: 180 - DNA, 24 bp, Artificial Sequence
  Description of Artificial Sequence Synthetic clone 4553 H1 oligonucleotide
ggcttcaacatcaaggacacctac

SEQ ID NO: 181 - protein, 13 aa, Artificial Sequence
  Description of Artificial Sequence Synthetic clone 4553 H3 peptide
SRWGGDGFYAMDY

SEQ ID NO: 182 - DNA, 39 bp, Artificial Sequence
  Description of Artificial Sequence Synthetic clone 4553 H3 oligonucleotide
agtcggtggggaggcgacggcttctacgctatggattat

SEQ ID NO: 183 - protein, 8 aa, Artificial Sequence
  Description of Artificial Sequence Synthetic clone 4553 H2 peptide
IYPTNGYT

SEQ ID NO: 184 - DNA, 24 bp, Artificial Sequence
  Description of Artificial Sequence Synthetic clone 4553 H2 oligonucleotide
atctatccaactaatggatacacc

SEQ ID NO: 185 - protein, 98 aa, Artificial Sequence; identical to SEQ ID NO: 67
  Description of Artificial Sequence Synthetic clone 4553 CH1 polypeptide

SEQ ID NO: 186 - DNA, 294 bp, Artificial Sequence
  Description of Artificial Sequence Synthetic clone 4553 CH1 polynucleotide
gcatctacaaaggggcctagtgtgtttccactggccccctctagtaaatccacctctgggggaacagcagccctgggatgtctggtgaaggactatttcccagagcccgtcactgtgagttggaactcaggcgccctgacatccggggtccatacttttcctgctgtgctgcagtcaagcggcctgtactctctgtcctctgtggtcaccgtgccaagttcaagcctggggactcagacctatatctgcaacgtgaatcacaagccaagcaatacaaaagtcgacaagaaagtg

SEQ ID NO: 187 - protein, 110 aa, Artificial Sequence; identical to SEQ ID NO: 69
  Description of Artificial Sequence Synthetic clone 4553 CH2 polypeptide

SEQ ID NO: 188 - DNA, 330 bp, Artificial Sequence
  Description of Artificial Sequence Synthetic clone 4553 CH2 polynucleotide
gcaccagagctgctgggaggaccatccgtgttcctgtttccacccaagcctaaagacaccctgatgatttccaggactccagaagtcacctgcgtggtcgtggacgtgtctcacgaggaccccgaagtcaagttcaactggtacgtggatggcgtcgaggtgcataatgccaagacaaaacccagggaggaacagtacaactcaacttatcgcgtcgtgagcgtcctgaccgtgctgcaccaggactggctgaacggcaaggagtataagtgcaaagtgagcaataaggctctgcccgcacctatcgagaaaaccattagcaaggccaaa

SEQ ID NO: 189 - protein, 106 aa, Artificial Sequence; identical to SEQ ID NO: 133
  Description of Artificial Sequence Synthetic clone 4553 CH3 polypeptide

SEQ ID NO: 190 - DNA, 318 bp, Artificial Sequence
  Description of Artificial Sequence Synthetic clone 4553 CH3 polynucleotide
gggcagcctagagaaccacaggtctacgtgtatcctccaagcagggacgagctgaccaagaaccaggtctccctgacatgtctggtgaaagggttttaccccagtgatatcgctgtggagtgggaatcaaatggacagcctgaaaacaattataagaccacaccccctgtgctggacagcgatggcagcttcgctctggtctccaagctgactgtggataaatctcggtggcagcagggcaacgtctttagttgttcagtgatgcatgaggcactgcacaatcattacacccagaagagcctgtccctgtctcccggc

SEQ ID NO: 191 - protein, 450 aa, Artificial Sequence
  Description of Artificial Sequence Synthetic clone 4555 Full polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 192 - DNA, 1350 bp, Artificial Sequence
  Description of Artificial Sequence Synthetic clone 4555 Full polynucleotide
gaagtccagctggtcgaaagcggaggaggactggtgcagccaggagggtctctgcgactgagttgcgccgcttcaggcttcaacatcaaggacacctacattcactgggtgcgccaggctcctggaaaaggcctggagtgggtggcacgaatctatccaaccaatggatacacacggtatgccgacagcgtgaagggccggttcaccattagcgcagatacttccaaaaacaccgcctacctgcagatgaacagcctgcgagccgaagataccgctgtgtactattgcagtcggtggggaggcgacggcttctacgctatggattattgggggcagggaacactggtcactgtgagctccgcatctactaaggggcctagtgtgtttccactggccccctctagtaaatccacatctgggggaactgcagccctgggatgtctggtgaaggactatttcccagagcccgtcacagtgagttggaactcaggcgccctgacttccggggtccatacctttcctgctgtgctgcagtcaagcggcctgtactctctgtcctctgtggtcacagtgccaagttcaagcctggggacccagacatatatctgcaacgtgaatcacaagccaagcaatactaaagtcgacaagaaagtggaacccaagagctgtgataaaactcatacctgcccaccttgtcctgcaccagagctgctgggaggaccatccgtgttcctgtttccacccaagcctaaagacaccctgatgatttccaggaccccagaagtcacatgcgtggtcgtggacgtgtctcacgaggaccccgaagtcaagttcaactggtacgtggatggcgtcgaggtgcataatgccaagacaaaacccagggaggaacagtacaactcaacatatcgcgtcgtgagcgtcctgactgtgctgcaccaggactggctgaacggcaaggagtataagtgcaaagtgagcaataaggctctgcccgcacctatcgagaaaaccattagcaaggctaaagggcagcctagagaaccacaggtctacgtgctgcctccaagcagggacgagctgacaaagaaccaggtctccctgctgtgtctggtgaaagggttctatcccagtgatatcgcagtggagtgggaatcaaatggacagcctgaaaacaattacctgacctggccccctgtgctggacagcgatggcagcttcttcctgtattccaagctgacagtggataaatctcggtggcagcagggcaacgtctttagttgttcagtgatgcatgaggccctgcacaatcattacacccagaagagcctgtccctgtctcccggcaaa

SEQ ID NO: 193 - protein, 120 aa, Artificial Sequence [sequence omitted]

SEQ ID NO: 194 - DNA, 360 bp, Artificial Sequence
  Description of Artificial Sequence Synthetic clone 4555 VH polynucleotide
gaagtccagctggtcgaaagcggaggaggactggtgcagccaggagggtctctgcgactgagttgcgccgcttcaggcttcaacatcaaggacacctacattcactgggtgcgccaggctcctggaaaaggcctggagtgggtggcacgaatctatccaaccaatggatacacacggtatgccgacagcgtgaagggccggttcaccattagcgcagatacttccaaaaacaccgcctacctgcagatgaacagcctgcgagccgaagataccgctgtgtactattgcagtcggtggggaggcgacggcttctacgctatggattattgggggcagggaacactggtcactgtgagctcc

SEQ ID NO: 195 - protein, 8 aa, Artificial Sequence
  Description of Artificial Sequence Synthetic clone 4555 H1 peptide
GFNIKDTY

SEQ ID NO: 196 - DNA, 24 bp, Artificial Sequence
  Description of Artificial Sequence Synthetic clone 4555 H1 oligonucleotide
ggcttcaacatcaaggacacctac

SEQ ID NO: 197 - protein, 13 aa, Artificial Sequence
  Description of Artificial Sequence Synthetic clone 4555 H3 peptide
SRWGGDGFYAMDY

SEQ ID NO: 198 - DNA, 39 bp, Artificial Sequence; identical to SEQ ID NO: 182
  Description of Artificial Sequence Synthetic clone 4555 H3 oligonucleotide

SEQ ID NO: 199 - protein, 8 aa, Artificial Sequence
  Description of Artificial Sequence Synthetic clone 4555 H2 peptide
IYPTNGYT

SEQ ID NO: 200 - DNA, 24 bp, Artificial Sequence
  Description of Artificial Sequence Synthetic clone 4555 H2 oligonucleotide
atctatccaaccaatggatacaca

SEQ ID NO: 201 - protein, 98 aa, Artificial Sequence; identical to SEQ ID NO: 67
  Description of Artificial Sequence Synthetic clone 4555 CH1 polypeptide

SEQ ID NO: 202 - DNA, 294 bp, Artificial Sequence
  Description of Artificial Sequence Synthetic clone 4555 CH1 polynucleotide
gcatctactaaggggcctagtgtgtttccactggccccctctagtaaatccacatctgggggaactgcagccctgggatgtctggtgaaggactatttcccagagcccgtcacagtgagttggaactcaggcgccctgacttccggggtccatacctttcctgctgtgctgcagtcaagcggcctgtactctctgtcctctgtggtcacagtgccaagttcaagcctggggacccagacatatatctgcaacgtgaatcacaagccaagcaatactaaagtcgacaagaaagtg

SEQ ID NO: 203 - protein, 110 aa, Artificial Sequence; identical to SEQ ID NO: 69
  Description of Artificial Sequence Synthetic clone 4555 CH2 polypeptide

SEQ ID NO: 204 - DNA, 330 bp, Artificial Sequence
  Description of Artificial Sequence Synthetic clone 4555 CH2 polynucleotide
gcaccagagctgctgggaggaccatccgtgttcctgtttccacccaagcctaaagacaccctgatgatttccaggaccccagaagtcacatgcgtggtcgtggacgtgtctcacgaggaccccgaagtcaagttcaactggtacgtggatggcgtcgaggtgcataatgccaagacaaaacccagggaggaacagtacaactcaacatatcgcgtcgtgagcgtcctgactgtgctgcaccaggactggctgaacggcaaggagtataagtgcaaagtgagcaataaggctctgcccgcacctatcgagaaaaccattagcaaggctaaa

SEQ ID NO: 205 - protein, 106 aa, Artificial Sequence
  Description of Artificial Sequence Synthetic clone 4555 CH3 polypeptide
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 206 - DNA, 318 bp, Artificial Sequence
  Description of Artificial Sequence Synthetic clone 4555 CH3 polynucleotide
gggcagcctagagaaccacaggtctacgtgctgcctccaagcagggacgagctgacaaagaaccaggtctccctgctgtgtctggtgaaagggttctatcccagtgatatcgcagtggagtgggaatcaaatggacagcctgaaaacaattacctgacctggccccctgtgctggacagcgatggcagcttcttcctgtattccaagctgacagtggataaatctcggtggcagcagggcaacgtctttagttgttcagtgatgcatgaggccctgcacaatcattacacccagaagagcctgtccctgtctcccggc

SEQ ID NO: 207 - protein, 232 aa, Artificial Sequence
  Description of Artificial Sequence Synthetic clone 4558 Full polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 208 - DNA, 696 bp, Artificial Sequence
  Description of Artificial Sequence Synthetic clone 4558 Full polynucleotide
gaacctaagagcagcgacaagactcacacctgcccaccttgtccagcaccagaactgctgggaggaccaagcgtgttcctgtttccacccaagcccaaagataccctgatgatcagccgaacacccgaagtgacttgcgtggtcgtggacgtgtcccacgaggaccccgaagtcaagttcaactggtacgtggacggcgtcgaagtgcataatgctaagacaaaaccacgggaggaacagtacaactctacttatagagtcgtgagtgtcctgaccgtgctgcatcaggattggctgaacggcaaagagtataagtgcaaagtgtctaataaggccctgcctgctccaatcgagaaaaccattagtaaggctaaagggcagcccagggaacctcaggtctacgtgtatcctccaagtcgcgacgagctgaccaagaaccaggtctcactgacatgtctggtgaaaggattttacccttccgatattgcagtggagtgggaatctaatggccagccagagaacaattataagaccacaccccctgtgctggacagcgatgggtccttcgcactggtctcaaagctgacagtggacaaaagcagatggcagcagggaaacgtctttagctgttccgtgatgcacgaagccctgcacaatcattacactcagaagtctctgagtctgtcacctggcaaa

SEQ ID NO: 209 - protein, 110 aa, Artificial Sequence; identical to SEQ ID NO: 69
  Description of Artificial Sequence Synthetic clone 4558 CH2 polypeptide

SEQ ID NO: 210 - DNA, 330 bp, Artificial Sequence
  Description of Artificial Sequence Synthetic clone 4558 CH2 polynucleotide
gcaccagaactgctgggaggaccaagcgtgttcctgtttccacccaagcccaaagataccctgatgatcagccgaacacccgaagtgacttgcgtggtcgtggacgtgtcccacgaggaccccgaagtcaagttcaactggtacgtggacggcgtcgaagtgcataatgctaagacaaaaccacgggaggaacagtacaactctacttatagagtcgtgagtgtcctgaccgtgctgcatcaggattggctgaacggcaaagagtataagtgcaaagtgtctaataaggccctgcctgctccaatcgagaaaaccattagtaaggctaaa

SEQ ID NO: 211 - protein, 106 aa, Artificial Sequence; identical to SEQ ID NO: 133
  Description of Artificial Sequence Synthetic clone 4558 CH3 polypeptide

SEQ ID NO: 212 - DNA, 318 bp, Artificial Sequence
  Description of Artificial Sequence Synthetic clone 4558 CH3 polynucleotide
gggcagcccagggaacctcaggtctacgtgtatcctccaagtcgcgacgagctgaccaagaaccaggtctcactgacatgtctggtgaaaggattttacccttccgatattgcagtggagtgggaatctaatggccagccagagaacaattataagaccacaccccctgtgctggacagcgatgggtccttcgcactggtctcaaagctgacagtggacaaaagcagatggcagcagggaaacgtctttagctgttccgtgatgcacgaagccctgcacaatcattacactcagaagtctctgagtctgtcacctggc

SEQ ID NO: 213 - protein, 481 aa, Artificial Sequence
  Description of Artificial Sequence Synthetic clone 719 Full polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDEDGSFALVSKLTVDKSRWQQGNVFSCSVMHEALHNHYTQKSLSLSPGK

SEQ ID NO: 214 - DNA, 1443 bp, Artificial Sequence
  Description of Artificial Sequence Synthetic clone 719 Full polynucleotide
gacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaaggtggttctggtggtggttctggtggtggttctggtggtggttctggtggtggttctggtgaagtgcagctggtggagtctgggggaggcttggtacagcctggcgggtccctgagactctcctgtgcagcctctggattcaacattaaagatacttatatccactgggtccggcaagctccagggaagggcctggagtgggtcgcacgtatttatcccacaaatggttacacacggtatgcggactctgtgaagggccgattcaccatctccgcagacacttccaagaacaccgcgtatctgcaaatgaacagtctgagagctgaggacacggccgtttattactgttcaagatggggcggagacggtttctacgctatggactactggggccaagggaccctggtcaccgtctcctcagccgccgagcccaagagcagcgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacacatacccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacgaggatggcagcttcgccctggtgtccaagctgacagtggataaatctcgatggcagcaggggaacgtgtttagttgttcagtgatgcatgaagccctgcacaatcattacactcagaagagcctgtccctgtctcccggcaaa

SEQ ID NO: 215 - protein, 107 aa, Artificial Sequence [sequence omitted]

SEQ ID NO: 216 - DNA, 321 bp, Artificial Sequence
  Description of Artificial Sequence Synthetic clone 719 VL polynucleotide
gacatccagatgacccagtctccatcctccctgtctgcatctgtaggagacagagtcaccatcacttgccgggcaagtcaggacgttaacaccgctgtagcttggtatcagcagaaaccagggaaagcccctaagctcctgatctattctgcatcctttttgtacagtggggtcccatcaaggttcagtggcagtcgatctgggacagatttcactctcaccatcagcagtctgcaacctgaagattttgcaacttactactgtcaacagcattacactaccccacccactttcggccaagggaccaaagtggagatcaaa

SEQ ID NO: 217 - protein, 6 aa, Artificial Sequence
  Description of Artificial Sequence Synthetic clone 719 L1 peptide
QDVNTA

SEQ ID NO: 218 - DNA, 18 bp, Artificial Sequence
  Description of Artificial Sequence Synthetic clone 719 L1 oligonucleotide
caggacgttaacaccgct

SEQ ID NO: 219 - protein, 9 aa, Artificial Sequence
  Description of Artificial Sequence Synthetic clone 719 L3 peptide
QQHYTTPPT

SEQ ID NO: 220 - DNA, 27 bp, Artificial Sequence
  Description of Artificial Sequence Synthetic clone 719 L3 oligonucleotide
caacagcattacactaccccacccact

SEQ ID NO: 221 - protein, 3 aa, Artificial Sequence
  Description of Artificial Sequence Synthetic clone 719 L2 peptide
SAS

SEQ ID NO: 222 - DNA, 9 bp, Artificial Sequence
  Description of Artificial Sequence Synthetic clone 719 L2 oligonucleotide
tctgcatcc

SEQ ID NO: 223 - protein, 120 aa, Artificial Sequence [sequence omitted]

SEQ ID NO: 224 - DNA, 360 bp, Artificial Sequence
  Description of Artificial Sequence Synthetic clone 719 VH polynucleotide
gaagtgcagctggtggagtctgggggaggcttggtacagcctggcgggtccctgagactctcctgtgcagcctctggattcaacattaaagatacttatatccactgggtccggcaagctccagggaagggcctggagtgggtcgcacgtatttatcccacaaatggttacacacggtatgcggactctgtgaagggccgattcaccatctccgcagacacttccaagaacaccgcgtatctgcaaatgaacagtctgagagctgaggacacggccgtttattactgttcaagatggggcggagacggtttctacgctatggactactggggccaagggaccctggtcaccgtctcctca

SEQ ID NO: 225 - protein, 8 aa, Artificial Sequence
  Description of Artificial Sequence Synthetic clone 719 H1 peptide
GFNIKDTY

SEQ ID NO: 226 - DNA, 24 bp, Artificial Sequence
  Description of Artificial Sequence Synthetic clone 719 H1 oligonucleotide
ggattcaacattaaagatacttat

SEQ ID NO: 227 - protein, 13 aa, Artificial Sequence
  Description of Artificial Sequence Synthetic clone 719 H3 peptide
SRWGGDGFYAMDY

SEQ ID NO: 228 - DNA, 39 bp, Artificial Sequence
  Description of Artificial Sequence Synthetic clone 719 H3 oligonucleotide
tcaagatggggcggagacggtttctacgctatggactac

SEQ ID NO: 229 - protein, 8 aa, Artificial Sequence
  Description of Artificial Sequence Synthetic clone 719 H2 peptide
IYPTNGYT

SEQ ID NO: 230 - DNA, 24 bp, Artificial Sequence
  Description of Artificial Sequence Synthetic clone 719 H2 oligonucleotide
atttatcccacaaatggttacaca

SEQ ID NO: 231 - protein, 110 aa, Artificial Sequence; identical to SEQ ID NO: 69
  Description of Artificial Sequence Synthetic clone 719 CH2 polypeptide

SEQ ID NO: 232 - DNA, 330 bp, Artificial Sequence; identical to SEQ ID NO: 70
  Description of Artificial Sequence Synthetic clone 719 CH2 polynucleotide

SEQ ID NO: 233 - protein, 106 aa, Artificial Sequence
  Description of Artificial Sequence Synthetic clone 719 CH3 polypeptide
GQPREPQVYTYPPSRDELTKNQVSLTCLVKGFYPSDIAVEWESNGQPENNYKTTPPVLDEDGSFALVSKLTVDKSRWQQGNVFSCSVMHEALHNHYTQKSLSLSPG

SEQ ID NO: 234 - DNA, 318 bp, Artificial Sequence
  Description of Artificial Sequence Synthetic clone 719 CH3 polynucleotide
ggccagccaagggagccccaggtgtacacatacccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacgaggatggcagcttcgccctggtgtccaagctgacagtggataaatctcgatggcagcaggggaacgtgtttagttgttcagtgatgcatgaagccctgcacaatcattacactcagaagagcctgtccctgtctcccggc

SEQ ID NO: 235 - protein, 232 aa, Artificial Sequence
  Description of Artificial Sequence Synthetic clone 4560 Full polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 236 - DNA, 696 bp, Artificial Sequence
  Description of Artificial Sequence Synthetic clone 4560 Full polynucleotide
gaacctaaaagcagcgacaagacccacacatgccccccttgtccagctccagaactgctgggaggaccaagcgtgttcctgtttccacccaagcccaaagatacactgatgatcagccgaactcccgaggtcacctgcgtggtcgtggacgtgtcccacgaggaccccgaagtcaagttcaactggtacgtggacggcgtcgaagtgcataatgcaaagactaaaccacgggaggaacagtacaactctacatatagagtcgtgagtgtcctgactgtgctgcatcaggattggctgaacggcaaagagtataagtgcaaagtgtctaataaggccctgcctgctccaatcgagaaaactattagtaaggcaaaagggcagcccagggaacctcaggtctacgtgctgcctccaagtcgcgacgagctgaccaagaaccaggtctcactgctgtgtctggtgaaaggattctatccttccgatattgccgtggagtgggaatctaatggccagccagagaacaattacctgacctggccccctgtgctggacagcgatgggtccttctttctgtattcaaagctgacagtggacaaaagcagatggcagcagggaaacgtctttagctgttccgtgatgcacgaagccctgcacaatcattacacccagaagtctctgagtctgtcacctggcaaa

SEQ ID NO: 237 - protein, 110 aa, Artificial Sequence; identical to SEQ ID NO: 69
  Description of Artificial Sequence Synthetic clone 4560 CH2 polypeptide

SEQ ID NO: 238 - DNA, 330 bp, Artificial Sequence
  Description of Artificial Sequence Synthetic clone 4560 CH2 polynucleotide
gctccagaactgctgggaggaccaagcgtgttcctgtttccacccaagcccaaagatacactgatgatcagccgaactcccgaggtcacctgcgtggtcgtggacgtgtcccacgaggaccccgaagtcaagttcaactggtacgtggacggcgtcgaagtgcataatgcaaagactaaaccacgggaggaacagtacaactctacatatagagtcgtgagtgtcctgactgtgctgcatcaggattggctgaacggcaaagagtataagtgcaaagtgtctaataaggccctgcctgctccaatcgagaaaactattagtaaggcaaaa

SEQ ID NO: 239 - protein, 106 aa, Artificial Sequence; identical to SEQ ID NO: 205
  Description of Artificial Sequence Synthetic clone 4560 CH3 polypeptide

SEQ ID NO: 240 - DNA, 318 bp, Artificial Sequence
  Description of Artificial Sequence Synthetic clone 4560 CH3 polynucleotide
gggcagcccagggaacctcaggtctacgtgctgcctccaagtcgcgacgagctgaccaagaaccaggtctcactgctgtgtctggtgaaaggattctatccttccgatattgccgtggagtgggaatctaatggccagccagagaacaattacctgacctggccccctgtgctggacagcgatgggtccttctttctgtattcaaagctgacagtggacaaaagcagatggcagcagggaaacgtctttagctgttccgtgatgcacgaagccctgcacaatcattacacccagaagtctctgagtctgtcacctggc

SEQ ID NO: 241 - protein, 214 aa, Artificial Sequence
  Description of Artificial Sequence Synthetic clone 4561 Full polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - DNA, 642 bp, Artificial Sequence
  Description of Artificial Sequence Synthetic clone 4561 Full polynucleotide
gatattcagatgacccagtcccctagctccctgtccgcttctgtgggcgacagggtcactatcacctgccgcgcatctcaggatgtgaacaccgcagtcgcctggtaccagcagaagcctgggaaagctccaaagctgctgatctacagtgcatcattcctgtattcaggagtgcccagccggtttagcggcagcagatctggcaccgacttcacactgactatctctagtctgcagcctgaggattttgccacatactattgccagcagcactataccacaccccctactttcggccaggggaccaaagtggagatcaagcgaactgtggccgctccaagtgtcttcatttttccacccagcgacgaacagctgaaatccggcacagcttctgtggtctgtctgctgaacaacttctaccccagagaggccaaagtgcagtggaaggtcgataacgctctgcagagtggcaacagccaggagagcgtgacagaacaggactccaaagattctacttatagtctgtcaagcaccctgacactgagcaaggcagactacgaaaagcataaagtgtatgcctgtgaggtgacccatcaggggctgtcttctcccgtgaccaagtctttcaaccgaggcgaatgt

SEQ ID NO: 243 - protein, 107 aa, Artificial Sequence [sequence omitted]

SEQ ID NO: 244 - DNA, 321 bp, Artificial Sequence
  Description of Artificial Sequence Synthetic clone 4561 VL polynucleotide
gatattcagatgacccagtcccctagctccctgtccgcttctgtgggcgacagggtcactatcacctgccgcgcatctcaggatgtgaacaccgcagtcgcctggtaccagcagaagcctgggaaagctccaaagctgctgatctacagtgcatcattcctgtattcaggagtgcccagccggtttagcggcagcagatctggcaccgacttcacactgactatctctagtctgcagcctgaggattttgccacatactattgccagcagcactataccacaccccctactttcggccaggggaccaaagtggagatcaag

SEQ ID NO: 245 - protein, 6 aa, Artificial Sequence
  Description of Artificial Sequence Synthetic clone 4561 L1 peptide
QDVNTA

SEQ ID NO: 246 - DNA, 18 bp, Artificial Sequence
  Description of Artificial Sequence Synthetic clone 4561 L1 oligonucleotide
caggatgtgaacaccgca

SEQ ID NO: 247 - protein, 9 aa, Artificial Sequence
  Description of Artificial Sequence Synthetic clone 4561 L3 peptide
QQHYTTPPT

SEQ ID NO: 248 - DNA, 27 bp, Artificial Sequence
  Description of Artificial Sequence Synthetic clone 4561 L3 oligonucleotide
cagcagcactataccacaccccctact

SEQ ID NO: 249 - protein, 3 aa, Artificial Sequence
  Description of Artificial Sequence Synthetic clone 4561 L2 peptide
SAS

SEQ ID NO: 250 - DNA, 9 bp, Artificial Sequence
  Description of Artificial Sequence Synthetic clone 4561 L2 oligonucleotide
agtgcatca

SEQ ID NO: 251 - protein, 107 aa, Artificial Sequence [sequence omitted]

SEQ ID NO: 252 - DNA, 321 bp, Artificial Sequence
  Description of Artificial Sequence Synthetic clone 4561 CL polynucleotide
cgaactgtggccgctccaagtgtcttcatttttccacccagcgacgaacagctgaaatccggcacagcttctgtggtctgtctgctgaacaacttctaccccagagaggccaaagtgcagtggaaggtcgataacgctctgcagagtggcaacagccaggagagcgtgacagaacaggactccaaagattctacttatagtctgtcaagcaccctgacactgagcaaggcagactacgaaaagcataaagtgtatgcctgtgaggtgacccatcaggggctgtcttctcccgtgaccaagtctttcaaccgaggcgaatgt

SEQ ID NO: 253 - protein, 448 aa, Artificial Sequence
  Description of Artificial Sequence Synthetic clone 3041 Full polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 254 - DNA, 1344 bp, Artificial Sequence
  Description of Artificial Sequence Synthetic clone 3041 Full polynucleotide
gaagtgcagctggtcgaatctggaggaggactggtgcagccaggagggtccctgcgcctgtcttgcgccgctagtggcttcacttttaccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctccgcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtggaacccaagtcttgtgataaaacccatacatgccccccttgtcctgcaccagagctgctgggaggaccaagcgtgttcctgtttccacccaagcctaaagatacactgatgattagtaggaccccagaagtcacatgcgtggtcgtggacgtgagccacgaggaccccgaagtcaagtttaactggtacgtggacggcgtcgaggtgcataatgccaagactaaacccagggaggaacagtacaacagtacctatcgcgtcgtgtcagtcctgacagtgctgcatcaggattggctgaacgggaaagagtataagtgcaaagtgagcaataaggctctgcccgcacctatcgagaaaacaatttccaaggcaaaaggacagcctagagaaccacaggtgtacgtgctgcctccatcaagggatgagctgacaaagaaccaggtcagcctgctgtgtctggtgaaaggattctatccctctgacattgctgtggagtgggaaagtaatggccagcctgagaacaattacctgacctggccccctgtgctggactcagatggcagcttctttctgtatagcaagctgaccgtcgacaaatcccggtggcagcaggggaatgtgtttagttgttcagtcatgcacgaggcactgcacaaccattacacccagaagtcactgtcactgtcaccaggg

SEQ ID NO: 255 - protein, 119 aa, Artificial Sequence [sequence omitted]

SEQ ID NO: 256 - DNA, 357 bp, Artificial Sequence
  Description of Artificial Sequence Synthetic clone 3041 VH polynucleotide
gaagtgcagctggtcgaatctggaggaggactggtgcagccaggagggtccctgcgcctgtcttgcgccgctagtggcttcacttttaccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctcc

SEQ ID NO: 257 - protein, 8 aa, Artificial Sequence
  Description of Artificial Sequence Synthetic clone 3041 H1 peptide
GFTFTDYT

SEQ ID NO: 258 - DNA, 24 bp, Artificial Sequence
  Description of Artificial Sequence Synthetic clone 3041 H1 oligonucleotide
ggcttcacttttaccgactacacc

SEQ ID NO: 259 - protein, 12 aa, Artificial Sequence
  Description of Artificial Sequence Synthetic clone 3041 H3 peptide
ARNLGPSFYFDY

SEQ ID NO: 260 - DNA, 36 bp, Artificial Sequence; identical to SEQ ID NO: 126
  Description of Artificial Sequence Synthetic clone 3041 H3 oligonucleotide

SEQ ID NO: 261 - protein, 8 aa, Artificial Sequence
  Description of Artificial Sequence Synthetic clone 3041 H2 peptide
VNPNSGGS

SEQ ID NO: 262 - DNA, 24 bp, Artificial Sequence
  Description of Artificial Sequence Synthetic clone 3041 H2 oligonucleotide
gtgaacccaaatagcggaggctcc

SEQ ID NO: 263 - protein, 98 aa, Artificial Sequence; identical to SEQ ID NO: 67
  Description of Artificial Sequence Synthetic clone 3041 CH1 polypeptide

SEQ ID NO: 264 - DNA, 294 bp, Artificial Sequence; identical to SEQ ID NO: 130
  Description of Artificial Sequence Synthetic clone 3041 CH1 polynucleotide

SEQ ID NO: 265 - protein, 110 aa, Artificial Sequence; identical to SEQ ID NO: 69
  Description of Artificial Sequence Synthetic clone 3041 CH2 polypeptide

SEQ ID NO: 266 - DNA, 330 bp, Artificial Sequence; identical to SEQ ID NO: 132
  Description of Artificial Sequence Synthetic clone 3041 CH2 polynucleotide

SEQ ID NO: 267 - protein, 106 aa, Artificial Sequence; identical to SEQ ID NO: 205
  Description of Artificial Sequence Synthetic clone 3041 CH3 polypeptide

SEQ ID NO: 268 - DNA, 318 bp, Artificial Sequence
  Description of Artificial Sequence Synthetic clone 3041 CH3 polynucleotide
ggacagcctagagaaccacaggtgtacgtgctgcctccatcaagggatgagctgacaaagaaccaggtcagcctgctgtgtctggtgaaaggattctatccctctgacattgctgtggagtgggaaagtaatggccagcctgagaacaattacctgacctggccccctgtgctggactcagatggcagcttctttctgtatagcaagctgaccgtcgacaaatcccggtggcagcaggggaatgtgtttagttgttcagtcatgcacgaggcactgcacaaccattacacccagaagtcactgtcactgtcaccaggg

SEQ ID NO: 269 - protein, 448 aa, Artificial Sequence
  Description of Artificial Sequence Synthetic clone 3057 Full polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 270 - DNA, 1344 bp, Artificial Sequence
  Description of Artificial Sequence Synthetic clone 3057 Full polynucleotide
gaagtgcagctggtcgaatctggaggaggactggtgcagccaggagggtccctgcgcctgtcttgcgccgctagtggcttcacttttaccgactacaccatggattgggtgcgacaggcacctggaaagggcctggagtgggtcgccgatgtgaacccaaatagcggaggctccatctacaaccagcggttcaagggccggttcaccctgtcagtggaccggagcaaaaacaccctgtatctgcagatgaatagcctgcgagccgaagatactgctgtgtactattgcgcccggaatctggggccctccttctactttgactattgggggcagggaactctggtcaccgtgagctccgcctccaccaagggaccttctgtgttcccactggctccctctagtaaatccacatctgggggaactgcagccctgggctgtctggtgaaggactacttcccagagcccgtcacagtgtcttggaacagtggcgctctgacttctggggtccacacctttcctgcagtgctgcagtcaagcgggctgtacagcctgtcctctgtggtcaccgtgccaagttcaagcctgggaacacagacttatatctgcaacgtgaatcacaagccatccaatacaaaagtcgacaagaaagtggaacccaagtcttgtgataaaacccatacatgccccccttgtcctgcaccagagctgctgggaggaccaagcgtgttcctgtttccacccaagcctaaagatacactgatgattagtaggaccccagaagtcacatgcgtggtcgtggacgtgagccacgaggaccccgaagtcaagtttaactggtacgtggacggcgtcgaggtgcataatgccaagactaaacccagggaggaacagtacaacagtacctatcgcgtcgtgtcagtcctgacagtgctgcatcaggattggctgaacgggaaagagtataagtgcaaagtgagcaataaggctctgcccgcacctatcgagaaaacaatttccaaggcaaaaggacagcctagagaaccacaggtgtacgtgtatcctccatcaagggatgagctgacaaagaaccaggtcagcctgacttgtctggtgaaaggattctatccctctgacattgctgtggagtgggaaagtaatggccagcctgagaacaattacaagaccacaccccctgtgctggactcagatggcagcttcgcgctggtgagcaagctgaccgtcgacaaatcccggtggcagcaggggaatgtgtttagttgttcagtcatgcacgaggcactgcacaaccattacacccagaagtcactgtcactgtcaccaggg

SEQ ID NO: 271 - protein, 119 aa, Artificial Sequence [sequence omitted]

SEQ ID NO: 272 - DNA, 357 bp, Artificial Sequence; identical to SEQ ID NO: 256
  Description of Artificial Sequence Synthetic clone 3057 VH polynucleotide

SEQ ID NO: 273 - protein, 8 aa, Artificial Sequence
  Description of Artificial Sequence Synthetic clone 3057 H1 peptide
GFTFTDYT

SEQ ID NO: 274 - DNA, 24 bp, Artificial Sequence
  Description of Artificial Sequence Synthetic clone 3057 H1 oligonucleotide
ggcttcacttttaccgactacacc

SEQ ID NO: 275 - protein, 12 aa, Artificial Sequence
  Description of Artificial Sequence Synthetic clone 3057 H3 peptide
ARNLGPSFYFDY

SEQ ID NO: 276 - DNA, 36 bp, Artificial Sequence; identical to SEQ ID NO: 126
  Description of Artificial Sequence Synthetic clone 3057 H3 oligonucleotide

SEQ ID NO: 277 - protein, 8 aa, Artificial Sequence
  Description of Artificial Sequence Synthetic clone 3057 H2 peptide
VNPNSGGS

SEQ ID NO: 278 - DNA, 24 bp, Artificial Sequence
  Description of Artificial Sequence Synthetic clone 3057 H2 oligonucleotide
gtgaacccaaatagcggaggctcc

SEQ ID NO: 279 - protein, 98 aa, Artificial Sequence; identical to SEQ ID NO: 67
  Description of Artificial Sequence Synthetic clone 3057 CH1 polypeptide

SEQ ID NO: 280 - DNA, 294 bp, Artificial Sequence; identical to SEQ ID NO: 130
  Description of Artificial Sequence Synthetic clone 3057 CH1 polynucleotide

SEQ ID NO: 281 - protein, 110 aa, Artificial Sequence; identical to SEQ ID NO: 69
  Description of Artificial Sequence Synthetic clone 3057 CH2 polypeptide

SEQ ID NO: 282 - DNA, 330 bp, Artificial Sequence; identical to SEQ ID NO: 132
  Description of Artificial Sequence Synthetic clone 3057 CH2 polynucleotide

SEQ ID NO: 283 - protein, 106 aa, Artificial Sequence; identical to SEQ ID NO: 133
  Description of Artificial Sequence Synthetic clone 3057 CH3 polypeptide

SEQ ID NO: 284 - DNA, 318 bp, Artificial Sequence; identical to SEQ ID NO: 134
  Description of Artificial Sequence Synthetic clone 3057 CH3 polynucleotide

SEQ ID NO: 285 - protein, 450 aa, Artificial Sequence; identical to SEQ ID NO: 175
  Description of Artificial Sequence Synthetic clone 1011 Full polypeptide

SEQ ID NO: 286 - DNA, 1350 bp, Artificial Sequence
  Description of Artificial Sequence Synthetic clone 1011 Full polynucleotide
gaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacgtgtacccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcgccctggtgtccaagctgacagtggataaatctcgatggcagcaggggaacgtgtttagttgttcagtgatgcatgaagccctgcacaatcattacactcagaagagcctgtccctgtctcccggcaaa

SEQ ID NO: 287 - protein, 120 aa, Artificial Sequence [sequence omitted]

SEQ ID NO: 288 - DNA, 360 bp, Artificial Sequence; identical to SEQ ID NO: 60
  Description of Artificial Sequence Synthetic clone 1011 VH polynucleotide

SEQ ID NO: 289 - protein, 8 aa, Artificial Sequence
  Description of Artificial Sequence Synthetic clone 1011 H1 peptide
GFNIKDTY

SEQ ID NO: 290 - DNA, 24 bp, Artificial Sequence
  Description of Artificial Sequence Synthetic clone 1011 H1 oligonucleotide
ggattcaacatcaaggacacctac

SEQ ID NO: 291 - protein, 13 aa, Artificial Sequence
  Description of Artificial Sequence Synthetic clone 1011 H3 peptide
SRWGGDGFYAMDY

SEQ ID NO: 292 - DNA, 39 bp, Artificial Sequence; identical to SEQ ID NO: 64
  Description of Artificial Sequence Synthetic clone 1011 H3 oligonucleotide

SEQ ID NO: 293 - protein, 8 aa, Artificial Sequence
  Description of Artificial Sequence Synthetic clone 1011 H2 peptide
IYPTNGYT

SEQ ID NO: 294 - DNA, 24 bp, Artificial Sequence
  Description of Artificial Sequence Synthetic clone 1011 H2 oligonucleotide
atctatcccactaatggatacacc

SEQ ID NO: 295 - protein, 98 aa, Artificial Sequence; identical to SEQ ID NO: 67
  Description of Artificial Sequence Synthetic clone 1011 CH1 polypeptide

SEQ ID NO: 296 - DNA, 294 bp, Artificial Sequence; identical to SEQ ID NO: 68
  Description of Artificial Sequence Synthetic clone 1011 CH1 polynucleotide

SEQ ID NO: 297 - protein, 110 aa, Artificial Sequence; identical to SEQ ID NO: 69
  Description of Artificial Sequence Synthetic clone 1011 CH2 polypeptide

SEQ ID NO: 298 - DNA, 330 bp, Artificial Sequence; identical to SEQ ID NO: 70
  Description of Artificial Sequence Synthetic clone 1011 CH2 polynucleotide

SEQ ID NO: 299 - protein, 106 aa, Artificial Sequence; identical to SEQ ID NO: 133
  Description of Artificial Sequence Synthetic clone 1011 CH3 polypeptide

SEQ ID NO: 300 - DNA, 318 bp, Artificial Sequence
  Description of Artificial Sequence Synthetic clone 1011 CH3 polynucleotide
ggccagccaagggagccccaggtgtacgtgtacccacccagcagagacgaactgaccaagaaccaggtgtccctgacatgtctggtgaaaggcttctatcctagtgatattgctgtggagtgggaatcaaatggacagccagagaacaattacaagaccacacctccagtgctggacagcgatggcagcttcgccctggtgtccaagctgacagtggataaatctcgatggcagcaggggaacgtgtttagttgttcagtgatgcatgaagccctgcacaatcattacactcagaagagcctgtccctgtctcccggc

SEQ ID NO: 301 - protein, 450 aa, Artificial Sequence; identical to SEQ ID NO: 191
  Description of Artificial Sequence Synthetic clone 1015 Full polypeptide

SEQ ID NO: 302 - DNA, 1350 bp, Artificial Sequence
  Description of Artificial Sequence Synthetic clone 1015 Full polynucleotide
gaggtgcagctggtggaaagcggaggaggactggtgcagccaggaggatctctgcgactgagttgcgccgcttcaggattcaacatcaaggacacctacattcactgggtgcgacaggctccaggaaaaggactggagtgggtggctcgaatctatcccactaatggatacacccggtatgccgactccgtgaaggggaggtttactattagcgccgatacatccaaaaacactgcttacctgcagatgaacagcctgcgagccgaagataccgctgtgtactattgcagtcgatggggaggagacggattctacgctatggattattggggacaggggaccctggtgacagtgagctccgcctctaccaagggccccagtgtgtttcccctggctccttctagtaaatccacctctggagggacagccgctctgggatgtctggtgaaggactatttccccgagcctgtgaccgtgagttggaactcaggcgccctgacaagcggagtgcacacttttcctgctgtgctgcagtcaagcgggctgtactccctgtcctctgtggtgacagtgccaagttcaagcctgggcacacagacttatatctgcaacgtgaatcataagccctcaaatacaaaagtggacaagaaagtggagcccaagagctgtgataagacccacacctgccctccctgtccagctccagaactgctgggaggacctagcgtgttcctgtttccccctaagccaaaagacactctgatgatttccaggactcccgaggtgacctgcgtggtggtggacgtgtctcacgaggaccccgaagtgaagttcaactggtacgtggatggcgtggaagtgcataatgctaagacaaaaccaagagaggaacagtacaactccacttatcgcgtcgtgagcgtgctgaccgtgctgcaccaggactggctgaacgggaaggagtataagtgcaaagtcagtaataaggccctgcctgctccaatcgaaaaaaccatctctaaggccaaaggccagccaagggagccccaggtgtacgtgctgccacccagcagagacgaactgaccaagaaccaggtgtccctgctgtgtctggtgaaaggcttctatcctagtgatattgctgtggagtgggaatcaaatggacagccagagaacaattacctgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggcaaa

SEQ ID NO: 303 - protein, 120 aa, Artificial Sequence [sequence omitted]

SEQ ID NO: 304 - DNA, 360 bp, Artificial Sequence; identical to SEQ ID NO: 60
  Description of Artificial Sequence Synthetic clone 1015 VH polynucleotide

SEQ ID NO: 305 - protein, 8 aa, Artificial Sequence
  Description of Artificial Sequence Synthetic clone 1015 H1 peptide
GFNIKDTY

SEQ ID NO: 306 - DNA, 24 bp, Artificial Sequence
  Description of Artificial Sequence Synthetic clone 1015 H1 oligonucleotide
ggattcaacatcaaggacacctac

SEQ ID NO: 307 - protein, 13 aa, Artificial Sequence
  Description of Artificial Sequence Synthetic clone 1015 H3 peptide
SRWGGDGFYAMDY

SEQ ID NO: 308 - DNA, 39 bp, Artificial Sequence; identical to SEQ ID NO: 64
  Description of Artificial Sequence Synthetic clone 1015 H3 oligonucleotide

SEQ ID NO: 309 - protein, 8 aa, Artificial Sequence
  Description of Artificial Sequence Synthetic clone 1015 H2 peptide
IYPTNGYT

SEQ ID NO: 310 - DNA, 24 bp, Artificial Sequence
  Description of Artificial Sequence Synthetic clone 1015 H2 oligonucleotide
atctatcccactaatggatacacc

SEQ ID NO: 311 - protein, 98 aa, Artificial Sequence; identical to SEQ ID NO: 67
  Description of Artificial Sequence Synthetic clone 1015 CH1 polypeptide

SEQ ID NO: 312 - DNA, 294 bp, Artificial Sequence; identical to SEQ ID NO: 68
  Description of Artificial Sequence Synthetic clone 1015 CH1 polynucleotide

SEQ ID NO: 313 - protein, 110 aa, Artificial Sequence; identical to SEQ ID NO: 69
  Description of Artificial Sequence Synthetic clone 1015 CH2 polypeptide

SEQ ID NO: 314 - DNA, 330 bp, Artificial Sequence; identical to SEQ ID NO: 70
  Description of Artificial Sequence Synthetic clone 1015 CH2 polynucleotide

SEQ ID NO: 315 - protein, 106 aa, Artificial Sequence; identical to SEQ ID NO: 205
  Description of Artificial Sequence Synthetic clone 1015 CH3 polypeptide

SEQ ID NO: 316 - DNA, 318 bp, Artificial Sequence
  Description of Artificial Sequence Synthetic clone 1015 CH3 polynucleotide
ggccagccaagggagccccaggtgtacgtgctgccacccagcagagacgaactgaccaagaaccaggtgtccctgctgtgtctggtgaaaggcttctatcctagtgatattgctgtggagtgggaatcaaatggacagccagagaacaattacctgacctggcctccagtgctggacagcgatggcagcttcttcctgtattccaagctgacagtggataaatctcgatggcagcaggggaacgtgtttagttgttcagtgatgcatgaagccctgcacaatcattacactcagaagagcctgtccctgtctcccggc

SEQ ID NO: 317 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK